(19) United States
(12) Patent Application Publication
(10) Pub. No.: US 2021/0161837 A1
(43) Pub. Date: Jun. 3, 2021

(54) METHODS FOR TREATING CANCER WITH RORGAMMA INHIBITORS

(71) Applicant: The Regents of the University of California, Oakland, CA (US)

(72) Inventors: Hongwu Chen, Davis, CA (US); June X. Zou, Davis, CA (US); Junjian Wang, Davis, CA (US); Demin Cai, Davis, CA (US); Yong Xu, Guangzhou (CN); Xiaoqian Xue, Guangzhou (CN); Yan Zhang, Guangzhou (CN)

(73) Assignees: The Regents of the University of California, Oakland, CA (US); Guangzhou Institutes of Biomedicine and Health, Guangzhou (CN); U.S. Government represented by the Department of Veterans Affairs, Washington, DC (US)

(21) Appl. No.: 16/917,818

(22) Filed: Jun. 30, 2020

Related U.S. Application Data
(63) Continuation of application No. 16/038,069, filed on Jul. 17, 2018, now abandoned.
Application No. 16/038,069 is a continuation of application No. PCT/US2017/013966, filed on Jan. 18, 2017.
(60) Provisional application No. 62/280,074, filed on Jan. 18, 2016.
(60) Provisional application No. 62/306,996, filed on Mar. 11, 2016.

(51) Int. Cl. A61K 31/167; C07C 205/11; A61K 31/58; A61K 31/277; A61K 31/4166; A61K 45/06; A61P 35/00; A61K 31/138; A61K 31/222; A61K 31/337

(52) U.S. Cl. CPC A61K 31/167; C07C 205/11; A61K 31/58; A61K 31/277; A61K 31/337; A61K 45/06; A61P 35/00; A61K 31/138; A61K 31/222; A61K 31/4166

(57) ABSTRACT

The present invention provides compositions, methods, and kits comprising one or more compounds of Formula I, such as XY018, alone or in combination with one or more anticancer drugs, such as an anti-androgen drug, that are useful for treating cancer, e.g., prostate cancer, such as castration-resistant prostate cancer (CRPC), and numerous other types of cancer including lung cancer, breast cancer, liver cancer, ovarian cancer, endometrial cancer, bladder cancer, colon cancer, gastric cancer, lymphoma, and glioma.

### CROSS-REFERENCES TO RELATED APPLICATIONS

The present application is a continuation of U.S. application Ser. No. 16/038,069, filed Jul. 17, 2018, which is a continuation of International Patent Application No. PCT/US2017/013966 filed Jan. 18, 2017, which claims the benefit of and priority to U.S. Provisional Application No. 62/280,074, filed Jan. 18, 2016, and U.S. Provisional Application No. 62/306,996, filed Mar. 11, 2016, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under Grant No. R01CA206222, awarded by the National Institutes of Health, and Grant No. I01BX002237, awarded by the U.S. Department of Veterans Affairs, Office of R&D. The government has certain rights in the invention.

### REFERENCE TO SUBMISSION OF A SEQUENCE LISTING AS A TEXT FILE

The Sequence Listing written in file Sequence_Listing_070772-222730US-1202005.txt created on Feb. 10, 2021 and is 1,109 bytes, machine format IBM-PC, MS-Windows operating system, is hereby incorporated by reference in its entirety for all purposes.

### BACKGROUND OF THE INVENTION

Cancer is a leading cause of death in the United States, and despite the development of various different treatment methods such as chemotherapy, radiation therapy, and hormone deprivation therapy, there is no 100% effective cure to these diseases. One of the reasons current cancer treatment methods do not result in eradication of the cancerous tissue in afflicted individuals is through the development of drug-resistance by the cancerous cells. Patients who exhibit drug resistance to particular cancer drug will have tumors that no longer react to the drug and can continue growing despite continued treatment.

Because drug resistance can be a common outcome during the course of administering a particular cancer therapy, it is important to continue developing new drugs and to identify new targets to treat cancer.

As such, there is currently a need in the art for new methods and compositions for treating cancer patients and patients with drug-resistant cancers. The present disclosure addresses these and other needs.

### BRIEF SUMMARY OF THE INVENTION

In a first aspect, the present invention provides a method for treating cancer in a subject, the method comprising administering to the subject an effective amount of one or more compounds according to Formula I:

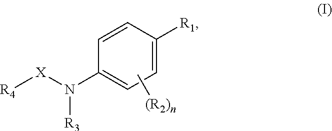

or a pharmaceutically acceptable salt, isomer, racemate, prodrug, co-crystalline complex, hydrate, or solvate thereof, wherein
X is C(═O) or SO₂;
n is an integer selected from the group consisting of 0, 1, 2, or 3;
R₁ is selected from the group consisting of H, halo, alkyl, trifluoromethyl, cyano, —COOR₅, —COR₅, —OR₅, —COH(CF₃)₂, heterocyclyl, and cycloalkyl,
wherein R₅ is selected from the group consisting of H, and C₁-C₃ alkyl group;
R₂ is selected from the group consisting of H, halogen, and alkyl;
R₃ is selected from the group consisting of H and alkyl;
R₄ is selected from the group consisting of C₀-C₄ alkylene-R₆, C₀-C₄ alkylene-R₇-cycloalkyl, and C₀-C₄ alkylene-R₇-heterocyclyl,
wherein R₆ is selected from the group consisting of —R₈, —OR₈, —COR₈, —COOR₈, —S(O)ₘR₈, cycloalkyl, and heterocyclyl, m is 0 or 2, and R₇ is selected from the group consisting of —OR₉, —C(O)R₉, —NR₉, —SR₉, —S(O)R₉, —S(O)₂R₉,
wherein R₈ is selected from the group consisting of H, and C₁-C₃ alkyl group, and R₉ is C₁-C₃ alkylene;
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of H, C₁-C₄ alkyl, and phenyl, and
R₁₁ is independently selected from the group consisting of C₁-C₄ alkyl, halogen, acetyl, methoxy, and ethoxy.

In some embodiments, the cancer is resistant to an anticancer drug. Non-limiting examples of anticancer drugs include anti-androgen drugs, chemotherapeutic agents, radiotherapeutic agents, antigen-specific immunotherapeutic agents, endocrine therapies, tyrosine kinase inhibitors, and combinations thereof. In certain instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the chemotherapeutic agent is tamoxifen, a taxane (e.g., paclitaxel and/or docetaxel), or combinations thereof.

In some embodiments, the cancer is selected from the group consisting of a prostate cancer, lung cancer, breast cancer, liver cancer, ovarian cancer, endometrial cancer, bladder cancer, colon cancer, gastric cancer, lymphoma, and glioma. In other embodiments, the subject is a mammal (e.g., human) in need of cancer treatment.

In certain embodiments, the prostate cancer is a castration-resistant prostate cancer. In particular embodiments, the castration-resistant prostate cancer is resistant to an anticancer drug such as, e.g., an anti-androgen drug and/or a taxane. In some instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the taxane is selected from the group consisting of paclitaxel, docetaxel, and combinations thereof.

In some embodiments, the lung cancer is a non-small-cell lung cancer (NSCLC), K-Ras mutant lung cancer, BRAF mutant lung cancer, EGFR mutant lung cancer, tyrosine kinase inhibitor-resistant lung cancer, or small cell lung cancer (SCLC).

In some embodiments, the breast cancer is a triple-negative breast cancer (TNBC), tamoxifen-resistant breast cancer, radiation-resistant breast cancer, HER2-positive breast cancer, or ER-positive breast cancer.

In some embodiments, the gastric cancer is an adenocarcinoma of the distal esophagus, gastroesophageal junction and/or stomach, a gastrointestinal carcinoid tumor, a gastrointestinal stromal tumor, an associated lymphoma, or a cancer linked to infection with H. pylori bacteria.

In another aspect, the present invention provides a method for treating a cancer in a subject, the method comprising administering to the subject an effective amount of one or more compounds according to Formula I:

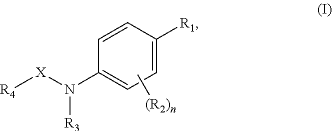

or a pharmaceutically acceptable salt, isomer, racemate, prodrug, co-crystalline complex, hydrate, or solvate thereof in combination with an effective amount of one or more anticancer drugs, wherein
X is C(═O) or SO₂;
n is an integer selected from the group consisting of 0, 1, 2, or 3;
R₁ is selected from the group consisting of H, halo, alkyl, trifluoromethyl, cyano, —COOR₅, —COR₅, —OR₅, —COH(CF₃)₂, heterocyclyl, and cycloalkyl,
wherein R₅ is selected from the group consisting of H, and C₁-C₃ alkyl group;
R₂ is selected from the group consisting of H, halogen, and alkyl;
R₃ is selected from the group consisting of H and alkyl;
R₄ is selected from the group consisting of C₀-C₄ alkylene-R₆, C₀-C₄ alkylene-R₇-cycloalkyl, and C₀-C₄ alkylene-R₇-heterocyclyl,
wherein R₁ is selected from the group consisting of —R₈, —OR₈, —COR₈, —COOR₈, —S(O)ₘR₈, cycloalkyl, and heterocyclyl, m is 0 or 2, and R₇ is selected from the group consisting of —OR₉, —C(O)R₉, —NR₉, —SR₉, —S(O)R₉, —S(O)₂R₉,
wherein R₈ is selected from the group consisting of H, and C₁-C₃ alkyl group, and R₉ is C₁-C₃ alkylene;
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of H, C₁-C₄ alkyl, and phenyl, and
R₁₁ is independently selected from the group consisting of C₁-C₄ alkyl, halogen, acetyl, methoxy, and ethoxy.

In some embodiments, the cancer is resistant to the anticancer drug. In particular embodiments, the compound of Formula I enhances the therapeutic effect of the anticancer drug. For example, the compound of Formula I can reverse or reduce cancer cell resistance to the anticancer drug and/or sensitize cancer cells to the anticancer drug.

In some embodiments, the cancer is selected from the group consisting of a prostate cancer, lung cancer, breast cancer, liver cancer, ovarian cancer, endometrial cancer, bladder cancer, colon cancer, gastric cancer, lymphoma, and glioma. In other embodiments, the subject is a mammal (e.g., human) in need of cancer treatment.

In certain embodiments, the prostate cancer is a castration-resistant prostate cancer. In particular embodiments, the castration-resistant prostate cancer is resistant to an anticancer drug such as, e.g., an anti-androgen drug and/or a taxane. In some instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the taxane is selected from the group consisting of paclitaxel, docetaxel, and combinations thereof.

In some embodiments, the lung cancer is a non-small-cell lung cancer (NSCLC), K-Ras mutant lung cancer, BRAF mutant lung cancer, EGFR mutant lung cancer, tyrosine kinase inhibitor-resistant lung cancer, or small cell lung cancer (SCLC).

In some embodiments, the breast cancer is a triple-negative breast cancer (TNBC), tamoxifen-resistant breast cancer, radiation-resistant breast cancer, HER2-positive breast cancer, or ER-positive breast cancer.

In some embodiments, the gastric cancer is an adenocarcinoma of the distal esophagus, gastroesophageal junction and/or stomach, a gastrointestinal carcinoid tumor, a gastrointestinal stromal tumor, an associated lymphoma, or a cancer linked to infection with H. pylori bacteria.

In some embodiments, the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof. In certain instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the chemotherapeutic agent is tamoxifen, a taxane (e.g., paclitaxel and/or docetaxel), or combinations thereof.

As a non-limiting example, a subject with a prostate cancer that is resistant to treatment with an anti-androgen drug such as enzalutamide can be administered the anti-androgen drug with an amount of a compound of Formula I sufficient to reverse or reduce prostate cancer cell resistance to the anti-androgen drug and/or sensitize the prostate cancer cells to the anti-androgen drug.

As another non-limiting example, a subject with a prostate cancer that is resistant to treatment with a taxane such as docetaxel can be administered the taxane with an amount of a compound of Formula I sufficient to reverse or reduce prostate cancer cell resistance to the taxane and/or sensitize the prostate cancer cells to the taxane.

As yet another non-limiting example, a subject with a breast cancer that is resistant to treatment with a chemotherapeutic agent such as tamoxifen can be administered the chemotherapeutic agent with an amount of a compound of Formula I sufficient to reverse or reduce breast cancer cell resistance to the chemotherapeutic agent and/or sensitize the breast cancer cells to the chemotherapeutic agent.

As a further non-limiting example, a subject with a breast cancer that is resistant to radiation treatment can be administered radiotherapy with an amount of a compound of Formula I sufficient to reverse or reduce breast cancer cell resistance to the radiotherapy and/or sensitize the breast cancer cells to the radiotherapy.

In yet another aspect, the present invention provides a composition comprising a compound of Formula I and an anticancer drug.

In some embodiments, the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof. In certain instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the chemotherapeutic agent is tamoxifen, a taxane (e.g., paclitaxel and/or docetaxel), or combinations thereof.

In some embodiments, the composition further comprises a pharmaceutically acceptable excipient or diluent. In other embodiments, the composition is formulated for oral or parenteral (e.g., intravenous) administration.

In still yet another aspect, the present invention provides a kit comprising a compound of Formula I and an anticancer drug.

In some embodiments, the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof. In certain instances, the anti-androgen drug is selected from the group consisting of enzalutamide, bicalutamide, arbiraterone, nilutamide, flutamide, apalutamide, finasteride, dutasteride, alfatradiol, and combinations thereof. In other instances, the chemotherapeutic agent is tamoxifen, a taxane (e.g., paclitaxel and/or docetaxel), or combinations thereof.

In some embodiments, the kit further comprises a label with instructions for administering the compound of Formula I and/or the anticancer drug to a subject. In certain instances, the subject is a mammal (e.g., human) in need of cancer treatment.

Other objects, features, and advantages of the present invention will be apparent to one of skill in the art from the following detailed description and figures.

### BRIEF DESCRIPTION OF THE DRAWINGS

FIG. 1 illustrates the NMR spectra of the compound XY018.

FIG. 2A illustrates that RORγ antagonist XY018 design was based, in part, on SR2211 and the GSK structures. The 2-([1,1′-biphenyl]-4-yl)-1,1,1,3,3,3-hexafluoropropan-2-ol group of SR2211 was kept, while the amide group of the GSK agonist was chosen as linker. FIG. 2B illustrates RORγ transcriptional activity measured by reporter gene assay in 293T cells with Gal4-RORγ LBD expression vectors. EC₅₀ values are reported as means±s.d. for ≥8 separate titrations from 10 μM. In vitro binding to the ligand binding domain (LBD) was measured by AlphaScreen and by thermal shift assay (TSA).

FIG. 3A illustrates XY018 docking into the RORγ ligand binding domain (LBD) by using Glide docking program with SP score. The predicted preferable binding mode is shown. Hydrogen bond interactions are shown as dash lines in red while π-π interaction is shown as dash line in green. For clarity, only the key residues in the pocket are shown. FIG. 3B illustrates that molecular dynamics demonstrated that the XY018 and RORγ complex is very stable with its predicted conformation. RMSD for ligand (black), Cα (red) and all atoms (blue) are shown for 200 ns simulation.

FIGS. 4A-4E illustrate that XY018 inhibits growth of CRPC cells. (a) Cell viability measured by Cell-Titer GLO of C4-2B cells treated with XY018, for 4 days. (b) Colony formation assay of C4-2B and 22Rv1 cells treated with vehicle or XY018. (c) Caspase-3/7 activities in C4-2B and 22Rv1 cells treated with vehicle or XY018 for 3 days. (d) TUNEL-positive apoptotic cells treated with vehicle or XY018 (5 μM) were counted and expressed as percentage of total cells. Data shown are mean percentage of apoptotic cells±s.d. (e) Immunoblotting analysis of indicated proteins in C4-2B cells treated with vehicle or XY018 for 3 days. Data shown are mean±s.d. Significance was calculated using Student's t-test. *p<0.05, **p<0.01.

FIGS. 5A-5G illustrate that XY018 inhibits growth and survival of CRPC cells. (a&b) C4-2B and 22Rv1 cell proliferation after the RORγ antagonist treatment. Cells were seeded in 6-well plates and counted after cells were treated with indicated concentrations of RORγ antagonists for 0, 2, 4 and 6 days by Coulter counter. Data are showed as mean±s.d. (c) Cell viability curves measured by CellTiter-GLO for different cells treated with RORγ antagonist XY018 or vehicle for 4 days. (d) Representative images of colony formation of C4-2B and 22Rv1 cells treated with vehicle, XY018 for 10 or 14 days. (e) representative images of TUNEL positive cells treated with vehicle or the antagonist (5 μM) in 22Rv1 cells are shown. (f) and (g) C4-2B and 22Rv1 cells were treated with vehicle, XY018. Three days later, cells were harvested for immunoblotting with indicated antibodies.

FIGS. 6A-6B illustrate that XY018 suppresses AR and its variant expression. (a) Immunoblotting of AR (full length) and AR variants in C4-2B or AR-V7 in VCaP cells treated with control or with XY018 at indicated concentrations for 72 hours. (b) qRT-PCR analysis of AR full-length and AR-V7 expression in VCaP cells treated with vehicle, 5 μM XY018 for 48 hours. Data shown are mean±s.d. Significance was calculated using Student's t-test. *p<0.05, **p<0.01.

FIG. 7 illustrates that XY018 inhibits AR and its variant expression in prostate cancer cells. Immunoblotting analysis of AR and its variant AR-V7 in PC346C, 22Rv1 and LAPC4 cells treated with vehicle or XY018 for 72 hours is shown.

FIGS. 8A-8B illustrate the in vivo effects of XY018 on growth of prostate cancer xenograft tumors and mouse body weight. (a) The effects of XY018 (20 mg/kg, i.p., 5 times a week) or vehicle treatment on growth of C4-2B xenografts are shown (n=6 mice per group). **p=9.92E-06, ***p=6.69E-05. (b) The effects of XY018 (5 mg/kg, i.p., 5 times a week) or vehicle treatment on growth of 22Rv1 xenografts are shown (n=6 mice per group). **p=2.7E-04, ***p=1.55E-05.

FIG. 9 illustrates the lack of inhibitor effects by XY018 on AR expression in non-malignant, human prostate epithelial cells. Immunoblotting analysis of RORγ and AR expression in non-malignant, human prostate epithelial RWPE1 and PZ-HPV7 cells within indicated treatments is shown.

FIG. 10A shows that XY018 strongly inhibits the growth and survival of numerous different types of breast cancer cells. FIG. 10B shows that XY018 potently inhibits the growth and survival of radiation-resistant breast cancer cells. FIG. 10C shows that XY018 displays strong inhibition of the growth and survival of tamoxifen-resistant breast cancer cells and is capable of sensitizing tamoxifen-resistant breast cancer cells to tamoxifen treatment.

FIG. 10D shows the IC50 values for XY018 and each breast cancer cell line.

FIG. 11A shows that XY018 strongly inhibits the growth and survival of numerous different types of lung cancer cells. FIG. 11B shows the IC50 values for XY018 and each lung cancer cell line.

FIGS. 12A-12H show the effect of XY018 on the growth and survival of (A) ovarian cancer cells, (B) bladder cancer cells, (C) endometrial cancer cells, (D) liver cancer cells, (E) glioblastoma cells, (F) lymphoma cells, (G) colon cancer cells, and (H) docetaxel-resistant prostate cancer cells. FIG. 12I shows the IC50 values for XY018 and each cancer cell line.

FIG. 13A shows cell viability, as measured by Cell-Titer GLO (Promega), of prostate cancer C4-2B cells treated with the indicated concentrations of various RORγ antagonists/inhibitors of Formula I (e.g., F17/No. 36, F18/No. 37 (XY018), F62/No. 80, F63/No. 81, F64/No. 82, F65/No. 83, and F68/No. 86) for 4 days. The “No.” label for each compound corresponds to the “Structure No.” in Table 2 (e.g., “No. 36”=“Structure No. 36” in Table 2). Experiments were independently performed three times. FIG. 13B shows the half-maximum inhibitory concentration (IC₅₀) for different RORγ inhibitors in C4-2B cell lines.

FIG. 14 shows cell viability, as measured by Cell-Titer GLO (Promega), of human gastric cancer KATO III cells treated with the indicated concentrations of RORγ antagonists/inhibitors F18/No. 37 (XY018), GSK9b, GSK805, SR2211, and GNE3500 for 4 days. Experiments were independently performed three times.

FIGS. 15A-15B show human triple-negative breast cancer (TNBC) MDA-MB468 and HCC70 cell lines treated with various RORγ antagonists/inhibitors of Formula I (e.g., F17/No. 36, F18/No. 37 (XY018), F62/No. 80, F63/No. 81, F64/No. 82, F65/No. 83, and F68/No. 86) for 4 days. The “No.” label for each compound corresponds to the “Structure No.” in Table 2 (e.g., “No. 36”=“Structure No. 36” in Table 2). The cell viability curves were measured by CellTiter-GLO. FIG. 15C shows half-maximum inhibitory concentration (IC₅₀) for different RORγ antagonists/inhibitors in MDA-MB468 and HCC70 cell lines.

FIG. 16A shows tumor volume, FIG. 16B shows weight, and FIG. 16C shows representative images of TNBC SUM159 orthotopic xenograft tumors in mammary fat pads from SCID mice treated with RORγ antagonists/inhibitors (F18/No. 37 (XY018), 5 mg/kg, i.p.; GSK805, 5 mg/kg, i.p.; GNE3500, 5 mg/kg, i.p.) or Vehicle (n=6 mice per group) for 45 days, 5 times per week. **p<0.01, *p<0.05.

### DETAILED DESCRIPTION OF THE INVENTION

### I. Introduction

The present disclosure relates to compounds of Formula I that are inhibitors of a nuclear receptor known as retinoic acid receptor-related orphan receptor γ (RORγ or RORgamma). In one aspect of this invention, it was surprisingly found that compounds of Formula I are useful in the treatment of cancer. In some embodiments, it was also found that compounds of Formula I can reverse or reduce cancer cell resistance to different classes of anticancer drugs and/or sensitize drug-resistant cancer cells to such anticancer drugs. Non-limiting examples of anticancer drugs that can be administered in combination with compounds of Formula I to enhance the therapeutic effect of the anticancer drug include anti-androgen drugs (e.g., bicalutamide, enzalutamide, arbiraterone, etc.), chemotherapeutic agents (e.g., tamoxifen and/or taxanes such as docetaxel), and combinations thereof.

Compounds of Formula I include compounds that inhibit RORγ transcription, translation, stability, and/or activity. Inhibition of RORγ activity can include inhibition of recruitment of coactivators such as SRC-1 and/or SRC-3 to an androgen receptor (AR) ROR response element (RORE). In some embodiments, inhibition of RORγ activity can include inhibition of transcription of the AR gene and/or a variant thereof such as AR-V7.

As described herein, the present inventors have found that compounds of Formula I strongly inhibit the growth of metastatic castration-resistant prostate cancer (mCRPC)-type prostate cancer (PCa) cells and xenograft tumors and induce marked cell death. The present inventors have also found that compounds of Formula I strongly inhibit the growth and survival of cancer cells of numerous other cancer types including lung cancer, breast cancer (e.g., triple-negative breast cancer), liver cancer, ovarian cancer, endometrial cancer, bladder cancer, colon cancer, gastric cancer, lymphoma, and glioblastoma multiforme.

### II. Definitions

It is noted here that as used in this specification and the appended claims, the singular forms “a,” “an,” and “the” include plural reference unless the context clearly dictates otherwise.

The term “RORγ” refers to either or both isoforms encoded by the RORC (RAR-related orphan receptor C) gene, namely RORγ (also referred to as RORγ1 or RORC1) and RORγt (also known as RORγ2 or RORC2).

The terms “subject”, “patient” or “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

As used herein, the term “effective amount” includes a dosage sufficient to produce a desired result with respect to the indicated disorder, condition, or mental state. The desired result may comprise a subjective or objective improvement in the recipient of the dosage. In one non-limiting example, an effective amount of a compound of Formula I includes an amount sufficient to alleviate the signs, symptoms, or causes of a cancer such as prostate cancer, e.g. CRPC. Thus, an effective amount can be an amount that slows or reverses tumor growth, increases mean time of survival, inhibits tumor progression or metastasis, or sensitizes a cancer cell to an anticancer drug to which it has become or is resistant. Also, in a second non-limiting example, an effective amount of a compound of Formula I includes an amount sufficient to cause a substantial improvement in a subject having cancer when administered to the subject. The amount will vary with the type of cancer being treated, the stage of advancement of the cancer, the type and concentration of composition applied, and the amount of anticancer drug (e.g., anti-androgen drug) that is also administered to the subject. In a third non-limiting example, an effective amount of a compound of Formula I can include an amount that is effective in enhancing the therapeutic activity of anticancer drugs such as anti-androgen drugs (e.g., bicalutamide, enzalutamide, arbiraterone, etc.) and/or chemotherapeutic agents (e.g., tamoxifen and/or taxanes such as docetaxel).

As used herein, the term “treating” includes, but is not limited to, methods and manipulations to produce beneficial changes in a recipient's health status, e.g., a patient's cancer status. The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors, or the resistance of the cancer cells to another cancer drug, then treatment of cancer has also been beneficial. Preventing the deterioration of a recipient's status is also included by the term. Treating, as used herein, also includes administering a compound of Formula I alone or in combination with an anticancer drug to a subject having cancer. In certain instances, the cancer is prostate cancer, lung cancer, breast cancer, liver cancer, ovarian cancer, endometrial cancer, bladder cancer, colon cancer, gastric cancer, lymphoma, or glioblastoma multiforme.

As used herein, the term “administering” includes activities associated with providing a patient an amount of a compound described herein, e.g., one or more compounds of Formula I. Administering includes providing unit dosages of compositions set forth herein to a patient in need thereof. Administering includes providing effective amounts of compounds, e.g., XY018, for a specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days, or in a specified sequence, e.g., administration of one or more compounds of Formula I followed by the administration of one or more anticancer drugs, or vice versa.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g., sequentially or simultaneously, via intravenous or intraperitoneal injection.

As used herein, the term “cancer” refers to conditions including solid cancers, lymphomas, and leukemias. Examples of different types of cancers include, but are not limited to, prostate cancer, lung cancer (e.g., non-small cell lung cancer or NSCLC), ovarian cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, endometrial cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. In some instances, the cancer can be metastatic. In certain instances, the cancer is prostate cancer, lung cancer, breast cancer, liver cancer, ovarian cancer, endometrial cancer, bladder cancer, colon cancer, gastric cancer, lymphoma, or a glioma such as glioblastoma multiforme. In other instances, the cancer can be resistant to an anticancer drug, e.g., an anti-androgen-resistant cancer, a taxane-resistant cancer (e.g., docetaxel-resistant cancer), a tamoxifen-resistant cancer, a radiation-resistant cancer, or a tyrosine kinase inhibitor-resistant cancer.

As used herein, the terms “prostate cancer” or “prostate cancer cell” refer to a cancer cell or cells that reside in prostate tissue. The prostate cancer can be benign, malignant, or metastatic. The prostate cancer can be androgen-insensitive, hormone-resistant, or castrate-resistant. The prostate cancer can be “advanced stage prostate cancer” or “advanced prostate cancer.” Advanced stage prostate cancer includes a class of prostate cancers that has progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, abiraterone-resistant prostate cancer, bicalutamide-resistant prostate cancer, etc.), taxane-resistant prostate cancer (e.g., docetaxel-resistant prostate cancer) and the like, hormone refractory prostate cancer, castration-resistant prostate cancer (CRPC), metastatic castration-resistant prostate cancer, AR-V7-induced drug-resistant prostate cancer such as AR-V7-induced anti-androgen-resistant prostate cancer (e.g., AR-V7-induced enzalutamide-resistant prostate cancer), AKR1C3-induced drug-resistant prostate cancer such as AKRC3-induced anti-androgen-resistant prostate cancer (e.g., AKR1C3-induced enzalutamide-resistant prostate cancer), and combinations thereof. In some instances, the advanced stage prostate cancers do not generally respond, or are resistant, to treatment with one or more of the following conventional prostate cancer therapies: enzalutamide, abiraterone, bicalutamide, and docetaxel. Compounds, compositions, and methods of the present invention are provided for treating prostate cancer, such as advanced stage prostate cancer, including any one or more (e.g., two, three, four, five, six, seven, eight, nine, ten, or more) of the types of advanced stage prostate cancers disclosed herein.

As used herein, the phrase “enhancing the therapeutic effects” includes any of a number of subjective or objective factors indicating a beneficial response or improvement of the condition being treated as discussed herein. For example, enhancing the therapeutic effects of an anticancer drug such as an anti-androgen drug (e.g., enzalutamide, abiraterone, or bicalutamide) or a chemotherapeutic agent such as tamoxifen or a taxane (e.g., docetaxel) includes reversing or reducing cancer cell resistance and/or sensitizing a drug-resistant cancer to anticancer drug therapy. Also, for example, enhancing the therapeutic effects of an anticancer drug includes altering drug-resistant cancer cells so that the cells are not resistant to the anticancer drug. Also, for example, enhancing the therapeutic effects of an anticancer drug includes additively or synergistically improving or increasing the activity of the anticancer drug. In some embodiments, the enhancement includes a one-fold, two-fold, three-fold, five-fold, ten-fold, twenty-fold, fifty-fold, hundred-fold, or thousand-fold increase in the therapeutic activity of an anticancer drug used to treat cancer.

As used herein, the phrase “reversing cancer cell resistance” includes altering or modifying a cancer cell that is resistant to anticancer drug therapy so that the cell is no longer resistant to anticancer drug therapy.

As used herein, the phrase “reducing cancer cell resistance” includes increasing the therapeutic activity of an anticancer drug towards cancer cells that are, or previously were, resistant to anticancer drug therapy.

As used herein, the phrase “sensitizing cancer cell resistance” includes inducing sensitization towards anticancer drug therapy in cancer cells which are resistant to anticancer drug therapy. Sensitization as used herein includes inducing the ability of a cancer cell to be effectively treated with an anticancer drug. Sensitization also includes reducing the dosage required to achieve a beneficial effect with an anticancer drug.

As used herein, the phrase “anti-androgen drug” includes anti-androgen compounds that alter the androgen pathway by blocking the androgen receptors, competing for binding sites on the cell's surface, or affecting or mediating androgen production. Anti-androgen drugs are useful for treating several diseases including, but not limited to, prostate cancer. Anti-androgen drugs include, but are not limited to, enzalutamide, abiraterone, bicalutamide, flutamide, nilutamide, apalutamide, finasteride, dutasteride, alfatradiol, and combinations thereof.

As used herein, the term “androgen receptor” or “AR” includes a nuclear receptor that binds androgenic hormones testosterone or dihydrotestosterone in the cytoplasm and translocates to the nucleus. AR modulates, inter alia, transcription of target genes by binding to Androgen Response Elements (AREs) in the promoters of such target genes.

As used herein, the term “AR variant” includes a splice variant of full-length AR. Various AR variants are known. See, Guo et al., Cancer Res., 69(6):2305-13 (2009). Exemplary AR variants include, but are not limited to, variants lacking a functional ligand binding domain (LBD). An example of an AR variant that lacks an LBD is AR-V7. “AR-V7” includes androgen receptor splice variant 7, a constitutively active variant of an AR that lacks a functional ligand binding domain (LBD). See, e.g., Hu et al., Cancer Research, 69(1):16-22 (2009).

“Pharmaceutically acceptable” or “therapeutically acceptable” includes a substance which does not interfere with the effectiveness or the biological activity of the active ingredients and which is not toxic to the hosts in the amounts used, and which hosts may be either humans or animals to which it is to be administered.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Heteroalkyl” refers to an alkyl group of any suitable length and having from 1 to 3 heteroatoms such as N, O and S. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers and alkyl-amines. The heteroatom portion of the heteroalkyl can replace a hydrogen of the alkyl group to form a hydroxy, thio or amino group. Alternatively, the heteroatom portion can be the connecting atom, or be inserted between two carbon atoms.

“Haloalkyl” refers to an alkyl group, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Heterocycloalkyl” refers to a saturated or partially unsaturated ring system having from 3 to 13 ring members and from 1 to 4 heteroatoms of N, O and S. Heterocycloalkyl groups can include fused bi- or tri-cyclic systems, and can include one or more points of unsaturation. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, 3 to 12 or 3 to 13 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, or morpholine. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl, oxo (═O), or aryl, among many others.

The heterocycloalkyl groups can be linked via any position on the ring. For example, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, and morpholine can be 1-, 2-, 3- or 4-morpholine.

### III. Description of the Embodiments

The present invention provides compositions, methods, and kits comprising one or more compounds of Formula I, alone or in combination with one or more anticancer drugs, such as an anti-androgen drug, that are useful for treating cancer, e.g., prostate cancer, such as castration-resistant prostate cancer (CRPC), and numerous other types of cancer including lung cancer, breast cancer, liver cancer, ovarian cancer, endometrial cancer, bladder cancer, colon cancer, gastric cancer, lymphoma, and glioma.

A. Compounds of Formula I

Compounds of Formula I (i.e., RORγ inhibitors or RORγ antagonists) include compounds that inhibit retinoic acid receptor-related orphan receptor γ (RORγ) transcription, translation, stability, and/or activity. In certain embodiments, compounds of Formula I bind to RORγ and inhibit the activity of the receptor. In other embodiments, compounds of Formula I selectively bind to RORγ and inhibit RORγ activity relative to RORα and/or RORβ. In some instances, inhibition of RORγ activity can include inhibition of recruitment of coactivators such as SRC-1 and/or SRC-3 to an androgen receptor (AR) ROR response element (RORE). In other instances, inhibition of RORγ activity can include inhibition of transcription of the AR gene and/or a variant thereof such as AR-V7.

In some embodiments, compounds of Formula I include inverse agonists that bind to RORγ and decrease its activity below a constitutive (e.g., intrinsic or basal) level activity in the absence of any ligand. In some embodiments, compounds of Formula I include pharmaceutically acceptable salts, derivatives, analogs, isomers, racemates, prodrugs, co-crystalline complexes, hydrates, and solvates thereof.

In certain embodiments, the compound of Formula I has a half-maximal inhibitory concentration (IC50) value of from about 100 nM to about 100 μM, e.g., from about 100 nM to about 50 μM from about 100 nM to about 25 μM, from about 100 nM to about 10 μM. from about 500 nM to about 100 μM, from about 500 nM to about 50 μM, from about 500 nM to about 25 μM, from about 500 nM to about 10 μM, from about 1 μM to about 100 AM, from about 1 μM to about 50 μM, from about 1 μM to about 25 μM, from about 1 μM to about 10 μM, or about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM. In some instances, the IC50 value for a specific compound of Formula I is measured using an in vitro assay in cancer cells that have been incubated with the compound. The IC50 value can be determined based on the effect of the compound of Formula I in inhibiting the survival of cancer cells such as cells from a cancer cell line or primary tumor cells. In other embodiments, the compound of Formula I has an inhibitor constant (Ki) that is essentially the same numerical value as the IC50 value, or is about one-half the value of the IC50 value.

In particular embodiments, the compound of Formula I has the following structure:

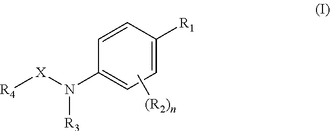

or a pharmaceutically acceptable salt, isomer, racemate, prodrug, co-crystalline complex, hydrate, or solvate thereof, wherein
X is C(═O) or SO₂;
n is an integer selected from the group consisting of 0, 1, 2, or 3;
R₁ is selected from the group consisting of H, halo, alkyl, trifluoromethyl, cyano, —COOR₅, —COR₅, —OR₅, —COH(CF₃)₂, heterocyclyl, and cycloalkyl,
wherein R₅ is selected from the group consisting of H, and C₁-C₃ alkyl group;
R₂ is selected from the group consisting of H, halogen, and alkyl;
R₃ is selected from the group consisting of H and alkyl;
R₄ is selected from the group consisting of C₀-C₄ alkylene-R₆, C₀-C₄ alkylene-R₇-cycloalkyl, and C₀-C₄ alkylene-R₇-heterocyclyl,
wherein R₆ is selected from the group consisting of —R₈, —OR₈, —COR₈, —COOR₈, —S(O)ₘR₈, cycloalkyl, and heterocyclyl, m is 0 or 2, and R₇ is selected from the group consisting of —OR₉, —C(O)R₉, —NR₉, —SR₉, —S(O)R₉, —S(O)₂R₉,
wherein R₈ is selected from the group consisting of H, and C₁-C₃ alkyl group, and R₉ is C₁-C₃ alkylene;
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-, bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of H, C₁-C₄ alkyl, and phenyl, and
R₁₁ is independently selected from the group consisting of C₁-C₄ alkyl, halogen, acetyl, methoxy, and ethoxy.

In particular embodiments, R₁ is selected from the group consisting of H, methyl, ethyl, propyl, fluoro, chloro, bromo, trifluoromethyl, cyano, —COH(CF₃)₂, —COOR₅, —COR₅, —OR₅, heterocyclyl, and cycloalkyl, wherein R₅ is selected from the group consisting of H, methyl, ethyl, and propyl.

In certain embodiments, R₂ is selected from the group consisting of H, methyl, ethyl, propyl, fluoro, chloro, and bromo.

In some embodiments, R₃ is selected from the group consisting of H, methyl, ethyl, propyl, and isopropyl.

In some embodiments, R₄ is selected from the group consisting of:
(1) C₀-C₄ alkylene-R₆, wherein R₆ is selected from the group consisting of —R₈, —OR₈, —COR₈, —COOR₅, —S(O)ₘR₈, cycloalkyl, and heterocyclyl, m is 0 or 2, and R₈ is selected from the group consisting of H, methyl, ethyl, and propyl group;
(2) C₀-C₄ alkylene-R₇-cycloalkyl,
wherein said cycloalkyl group is selected from the group consisting of cyclobutane, cyclopentane, cyclohexane, cycloheptane, phenyl, and naphthyl, and said cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂, wherein R₁₀ is selected from the group consisting of methyl, ethyl, propyl, isopropyl, and phenyl;
(3) C₀-C₄ alkylene-R₇-heterocyclyl,
wherein said heterocyclyl group is selected from the group consisting of imidazolyl, triazolyl, pyrazolyl, thienyl, oxazolyl, isoxazolyl, pyrazinyl, pyridazinyl, pyrimidinyl, pyrrolyl, piperazinyl, tetrahydro-pyrrolyl, piperidinyl, morpholinyl, 1,3-dioxolanyl, isoquinolinyl, indoline group, 1H-indazolyl, 1H-benzo [d] imidazolyl, 1H-indolyl, benzo [d] [1,3] dioxole, benzo [d] thiazolyl, and a member of the moieties shown in Table 1, and said heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, and a substituted pyridyl group;
wherein R₇ is selected from the group consisting of —OR₉, —C(O)R₉, —NR₉, —SR₉, —S(O)R₉, —S(O)₂R₉,
R₉ is C₁-C₃ alkylene,
R₁₀ is independently selected from the group consisting of H, methyl, ethyl, propyl, isopropyl, and phenyl,
R₁₁ is independently selected from the group consisting of methyl, ethyl, propyl, isopropyl, fluoro, chloro, bromo, acetyl, methoxy, and ethoxy, and
R₁₂ is selected from the group consisting of H, methyl, ethyl, propyl, and isopropyl.

TABLE 1
Possible Heterocyclic Moieties

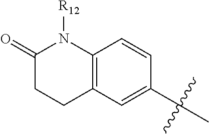

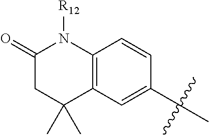

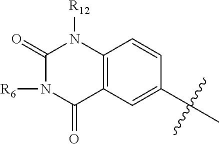

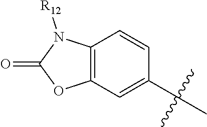

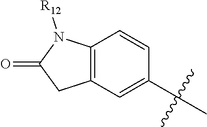

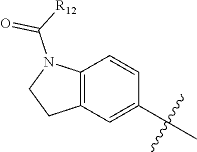

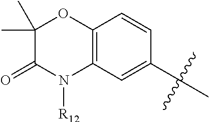

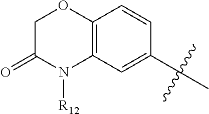

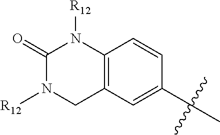

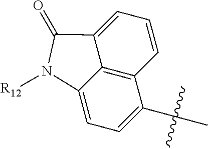

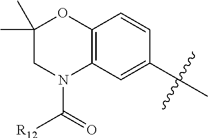

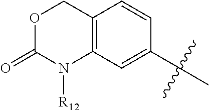

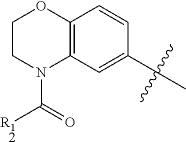

In particular embodiments, the RORγ inhibitor compound of Formula I is represented by a compound according to Formula Ia:

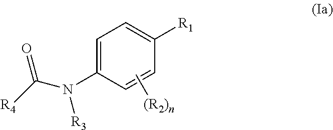

wherein
R₁ is selected from the group consisting of H, methyl, ethyl, propyl, fluoro, chloro, bromo, trifluoromethyl, cyano, —COH(CF₃)₂, —COOR₅, —COR₅, —OR₅, heterocyclyl, and cycloalkyl, wherein R₅ is selected from the group consisting of H, and C₁₋₃alkyl group;
R₂ is selected from the group consisting of H, fluoro, chloro, bromo;
R₃ is selected from the group consisting of H, methyl, ethyl, propyl, and isopropyl;
R₄ is selected from the group consisting of
(1) C₀-C₄ alkylene-R₆, wherein R₆ is selected from the group consisting of —R₈, —OR₈, —COR₈, —COOR₈, —S(O)ₘR₈, cycloalkyl, and heterocyclyl, m is 0 or 2, and R₈ is selected from the group consisting of H, and C₁₋₃alkyl;
(2) C₀-C₄ alkylene-R₇-cycloalkyl, wherein R₇ is selected from the group consisting of —OR₉, —C(O)R₉, —NR₉, —SR₉, —S(O)R₉, —S(O)₂R₉, wherein
R₉ is selected from the group consisting of C₁₋₃alkylene, heterocyclyl, and cycloalkyl,
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of C₁-C₄ alkyl, and phenyl, and
R₁₁ is independently selected from the group consisting of C₁-C₄ alkyl, halogen, acetyl, methoxy, and ethoxy.

In particular embodiments, R₁ is a cycloalkyl group having an unsaturated ring structure of 6 carbon atoms (e.g., a phenyl group), wherein the cycloalkyl group is substituted with a fluoro group (e.g., at the 2′ position) and a 1,1,1,3,3,3-hexafluoro-2-hydroxypropan-2-yl group (e.g., at the 4′ position). In some embodiments, R₂ is H. In other embodiments, R₃ is H.

In certain embodiments, the RORγ inhibitor compound of Formula I is represented by a compound according to Formula Ib:

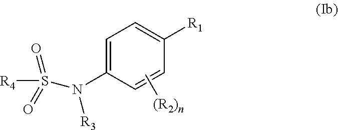

wherein
R₁ is selected from the group consisting of H, methyl, ethyl, propyl, fluoro, chloro, bromo, trifluoromethyl, cyano, —COH(CF₃)₂, —COOR₅, —COR₅, —OR₅, heterocyclyl, and cycloalkyl, wherein
R₅ is selected from the group consisting of H, and C₁₋₃alkyl group, and said cycloalkyl group is a saturated or unsaturated ring structure ranging from 3 to 10 carbon atoms,
said cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
said heterocyclyl group is a 5 to 12 membered saturated or unsaturated mono-, bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group;
R₂ is selected from the group consisting of H, fluoro, chloro, bromo;
R₃ is selected from the group consisting of H, methyl, ethyl, propyl, and isopropyl;
R₄ is selected from the group consisting of
(1) C₀-C₄ alkylene-R₆, wherein R₆ is selected from the group consisting of —R₈, —OR₈, and cycloalkyl, and R₈ is selected from the group consisting of H, and C₁₋₃alkyl;
(2) C₀-C₄ alkylene-R₇-heterocyclyl, wherein R₇ is selected from the group consisting of —OR₉, —C(O)R₉, —NR₉, —SR₉, —S(O)R₉, —S(O)₂R₉, and R₉ is selected from the group consisting of C₁₋₃alkylene, heterocyclyl, and cycloalkyl,
wherein said heterocyclyl group is selected from the group consisting of imidazolyl, triazolyl, pyrazolyl, thienyl, oxazolyl, isoxazolyl, pyrazinyl, pyridazinyl, pyrimidinyl, pyrrolyl, piperazinyl, tetrahydro-pyrrolyl, piperidinyl, morpholinyl, 1,3-dioxolanyl, isoquinolinyl, indoline group, 1H-indazolyl, 1H-benzo [d] imidazolyl, 1H-indolyl, benzo [d][1,3]dioxole, benzo [d] thiazolyl, and a member of the moieties shown in Table 1, and said heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group;
R₁₀ is independently selected from the group consisting of C₁₋₄alkyl and phenyl,
R₁₁ is independently selected from the group consisting of methyl, ethyl, propyl, isopropyl, halogen, acetyl, methoxy, and ethoxy, and
R₁₂ is selected from the group consisting of H, methyl, ethyl, propyl, isopropyl.

In particular embodiments, the compound of Formula I is selected from the group consisting of 1-ethyl-N-(2-fluoro-4-(1,1,1,3,3,3-hexafluoro-2-hydroxy-2-yl) phenyl)-2-oxo-1, 2-dihydro-benzo [cd] indole-6-sulfonamide, N-(2-fluoro-4-(1,1,1,3,3,3-hexafluoro-2-hydroxy-2-yl) phenyl)-4,4-dimethyl-2-oxo-1,2,3,4-tetrahydro-quinolin-6-sulfonamide, N-(2-fluoro-4-(1,1,1,3,3,3-hexafluoro-2-hydroxy-2-yl) phenyl)-2-oxo-1-propyl-1,2,3,4-tetrahydro-quinolin-6-sulfonamide, N-(2-fluoro-4-(1,1,1,3,3,3-hexafluoro-2-hydroxy-2-yl) phenyl)-2-oxo-3-propyl-2, 3-benzo [d] oxazole-6-sulfonamide, N-(2-fluoro-4-(1,1,1,3,3,3-hexafluoro-2-hydroxy-2-yl) phenyl)-2-oxo-indoline-5-sulfonamide, N-(2-fluoro-4-(1,1,1,3,3,3-hexafluoro-2-hydroxy-2-yl) phenyl)-2,4-dioxo-1,3-propyl-1,2,3,4-tetrahydro-quinazolin-6-sulfonamide, N-(2-fluoro-4-(1,1,1,3,3,3-hexafluoro-2-hydroxy-2-yl) phenyl)-2-oxo-1,2,3,4-tetrahydro-quinolin-6-sulfonamide, 1-ethyl-N-(2-fluoro-4-(1,1,1,3,3,3-hexafluoro-2-hydroxy-2-yl)-[1,1′-biphenyl] 4-yl)-2-oxo-1,2-dihydro-benzo [cd]indole-6-sulfonamide, N-(2-fluoro-4-(1,1,1,3,3,3-hexafluoro-2-hydroxy-2-yl)-[1,1′-biphenyl]-4-yl) 4,4-dimethyl-2-oxo-1,2,3,4-tetrahydro-quinolin-6-sulfonamide, N-(2-fluoro-4-(1,1,1,3,3,3-hexafluoro-2-hydroxy-2-yl) phenyl)-[1,1′-biphenyl]-4-yl)-2-oxo-1-propyl-1,2,3,4-tetrahydro-quinolin-6-sulfonamide, N-(2-fluoro-4-(1,1,1,3,3,3-hexafluoro-2-hydroxy-2-yl)-[1,1′-biphenyl]-4-yl) 2-oxo-3-propyl-2,3-benzo [d] oxazole-6-sulfonamide, N-(2-fluoro-4-(1,1,1,3,3,3-hexafluoro-2-hydroxy-2-yl)-[1,1′-biphenyl]-4-yl) 2-oxo-indoline-5-sulfonamide, N-(2-fluoro-4-(1,1,1,3,3,3-hexafluoro-2-hydroxy-2-yl)-[1,1′-biphenyl]-4-yl) 2,4-dioxo-1,3-dipropyl-1,2,3,4-tetrahydro-quinazolin-6-sulfonamide, N-(2-fluoro-4-(1,1,1,3,3,3-hexafluoro-2-hydroxy-2-yl)-[1,1′-biphenyl]-4-yl) 2,4-dioxo-1,2,3,4-tetrahydro-quinazolin-6-sulfonamide, N-(2-fluoro-4-(1,1,1,3,3,3-hexafluoro-2-hydroxy-2-yl)-[1,1′-biphenyl]-4-yl) 2-oxo-2,3-benzo [d] oxazole-6-sulfonamide, N-(2-fluoro-4-(1,1,1,3,3,3-hexafluoro-2-hydroxy-2-yl)-[1,1′-biphenyl]-4-yl) 2-oxo-1,2,3,4-tetrahydro-quinolin-6-sulfonamide, N-(2-fluoro-4-(1,1,1,3,3,3-hexafluoro-2-hydroxy-2-yl)-[1,1′-biphenyl]-4-yl) 3-methyl-2-oxo-1,2,3,4-tetrahydro-quinazolin-6-sulfonamide, 1-acetyl-N-(2′-fluoro-4′-(1,1,1,3,3,3-hexafluoro-2-hydroxy-2-yl)-[1,1′-phenyl]-4-yl) indoline-5-sulfonamide, N-(2′-fluoro-4′-(1,1,1,3,3,3-hexafluoro-2-hydroxy-2-yl)-[1,1′-biphenyl]-4 yl)-2,2-dimethyl-3-oxo-3,4-dihydro-2H-benzo [b] [1,4] oxazine-6-sulfonamide, 4-acetyl-N-(2′-fluoro-4′-(1,1,1,3,3,3-hexafluoro-2-hydroxy-2-yl)-[1,1′-phenyl]-4-yl)-2,2-dihydro-3,4-dihydro-2H-benzo [b] [1,4] oxazine-6-sulfonamide, N-(2′-fluoro-4′-(1,1,1,3,3,3-hexafluoro-2-hydroxy-2-yl)-[1,1′-biphenyl]-4 yl)-3-oxo-3,4-dihydro-2H-benzo [b] [1,4]oxazine-6-sulfonamide, N-(2′-fluoro-4′-(1,1,1,3,3,3-hexafluoro-2-hydroxy-2-yl)-[1,1′-biphenyl]-4 yl)-2-oxo-2,4-dihydro-1H-benzo [d] [1,3] oxazin-7-sulfonamide, N-(2′-fluoro-4′-(1,1,1,3,3,3-hexafluoro-2-hydroxy-2-yl)-[1,1′-biphenyl]-4 yl)-4-methyl-3-oxo-3,4-dihydro-2H-benzo [b] [1,4] oxazine-6-sulfonamide, 4-acetyl-N-(2′-fluoro-4′-(1,1,1,3,3,3-hexafluoro-2-hydroxy-2-yl)-[1,1′-phenyl]-4-yl)-3,4-dihydro-2H-benzo [b][1,4] oxazine-6-sulfonamide, 4-fluoro-N-(2′-fluoro-4′-(1,1,1,3,3,3-hexafluoro-2-hydroxy-2-yl)-[1,1′-biphenyl]-4-yl) benzenesulfonamide, 2,4-Difluoro-N-(2′-fluoro-4′-(1,1,1,3,3,3-hexafluoro-2-hydroxy-2-yl)-[1,1′-biphenyl]-4-yl) benzenesulfonamide, N-(2′-fluoro-4′-(1,1,1,3,3,3-hexafluoro-2-hydroxy-2-yl)-[1,1′-biphenyl]-4 yl) thiophene-2-sulfonamide, N-(2′-fluoro-4′-(1,1,1,3,3,3-hexafluoro-2-hydroxy-2-yl)-[1,1′-biphenyl]-4 yl)-2,3-dihydro-benzo [b] [1,4] dioxin-6-sulfonamide, N-(2′-fluoro-4′-(1,1,1,3,3,3-hexafluoro-2-hydroxy-2-yl)-[1,1′-biphenyl]-4 yl)-4-phenoxy-benzenesulfonamide, N-(2′-fluoro-4′-(1,1,1,3,3,3-hexafluoro-2-hydroxy-2-yl)-[1,1′-biphenyl]-4 yl)-4-phenyl-morpholin-benzenesulfonamide. N-(2′-fluoro-4′-(1,1,1,3,3,3-hexafluoro-2-hydroxy-2-yl)-[1,1′-biphenyl]-4-yl)-2-(p-toluene-yl) acetamide, 2-(3,4-dimethoxyphenyl)-N-(2′-fluoro-4′-(1,1,1,3,3,3-hexafluoro-2-hydroxy-2-yl)-[1,1′-biphenyl]-4-yl)-2-acetamide, 2-(4-chlorophenyl)-N-(2′-fluoro-4′-(1,1,1,3,3,3-hexafluoro-2-hydroxy-2-yl)-[1, 1′-biphenyl]-4-yl)-2-acetamide, 2-(4-bromophenyl)-N-(2′-fluoro-4′-(1,1,1,3,3,3-hexafluoro-2-hydroxy-2-yl)-[1, 1′-biphenyl]-4-yl)-2-acetamide, 2-(3-chlorophenyl)-N-(2′-fluoro-4′-(1,1,1,3,3,3-hexafluoro-2-hydroxy-2-yl)-[1, 1′-biphenyl]-4-yl)-2-acetamide, N-(2′-fluoro-4′-(1,1,1,3,3,3-hexafluoro-2-hydroxy-2-yl)-[1,1′-biphenyl]-4-yl)-2-(4-nitrophenyl) acetamide, N-(2′-fluoro-4′-(1,1,1,3,3,3-hexafluoro-2-hydroxy-2-yl)-[1,1′-biphenyl]-4-yl)-2-(2-nitrophenyl) acetamide, N-(2′-fluoro-4′-(1,1,1,3,3,3-hexafluoro-2-hydroxy-2-yl)-[1,1′-biphenyl]-4-yl)-2-(4-(trifluoromethyl) phenyl) acetamide, N-(2′-fluoro-4′-(1,1,1,3,3,3-hexafluoro-2-hydroxy-2-yl)-[1,1′-biphenyl]-4-yl)-pentanamide, N-(2′-fluoro-4′-(1,1,1,3,3,3-hexafluoro-2-hydroxy-2-yl)-[1,1′-biphenyl]-4-yl)-3,3-dimethylbutanamide, 2-(3,4-dichlorophenyl)-N-(2′-fluoro-4′-(1,1,1,3,3,3-hexafluoro-2-hydroxy-2-yl)-[1,1′-biphenyl]-4-yl) acetamide, N-(2′-fluoro-4′-(1,1,1,3,3,3-hexafluoro-2-hydroxy-2-yl)-[1,1′-biphenyl]-4-yl) benzamide, N-(2′-fluoro-4′-(1,1,1,3,3,3-hexafluoro-2-hydroxy-2-yl)-[1,1′-biphenyl]-4-yl)-2-(p-tolyl) benzenesulfonamide, 4-((N-(2-fluoro-4-(1,1,1,3,3,3-hexafluoro-2-hydroxy-2-yl) phenyl) sulfamoyl) methyl) benzoic acid methyl, 4-((N-(2-fluoro-4-(1,1,1,3,3,3-hexafluoro-2-hydroxy-2-yl)-[1,1′-biphenyl]-4-yl) sulfamoyl) methyl) benzoic acid, 4-((N-(2-fluoro-4-(1,1,1,3,3,3-hexafluoro-2-hydroxy-2-yl) phenyl) sulfamoyl) methyl) benzoic acid, 4-((N-(2-fluoro-4-(1,1,1,3,3,3-hexafluoro-2-hydroxy-2-yl)-[1,1′-biphenyl]-4-yl)-N-propyl-sulfamoyl-yl) methyl) benzoate, 4-((N-([1,1′-biphenyl]-4-yl) sulfamoyl) methyl) benzoate 4-((N-(4-acetylphenyl) sulfamoyl) methyl) benzoate, 4-((N-(3,4-dimethoxyphenyl) amino) methyl) benzoate, 4-((N-(2-oxo-1-propyl-1,2,3,4-tetrahydro-quinolin-6-yl) sulfamoyl) methyl) benzoic acid, N-(2′-fluoro-4′-(1,1,1,3,3,3-hexafluoro-2-hydroxy-2-yl)-[1,1′-biphenyl]-4-yl)-1-(4-(methylsulfonyl) phenyl) methanesulfonamide, N-(2′-fluoro-4′-(1,1,1,3,3,3-hexafluoro-2-hydroxy-2-yl)-[1,1′-biphenyl]-4-yl)-4-(trifluoromethyl) benzamide, 2-((N-(2′-fluoro-4′-(1,1,1,3,3,3-hexafluoro-2-hydroxy-2-yl)-[1,1′-biphenyl]4-yl) sulfamoyl) methyl) benzoate, N-(2′-fluoro-4′-(1,1,1,3,3,3-hexafluoro-2-hydroxy-2-yl)-[1,1′-biphenyl]-4-yl)-1-(3-nitrophenyl) methyl sulfonamide, 3-((N-(2′-fluoro-4′-(1,1,1,3,3,3-hexafluoro-2-hydroxy-2-yl)-[1,1′-biphenyl] 4-yl) sulfamoyl) methyl) benzoate, 1-(4-cyanophenyl)-N-(2′-fluoro-4′-(1,1,1,3,3,3-hexafluoro-2-hydroxy-2-yl)-[1,1′-biphenyl]-4-yl) methyl sulfonamide, N-(2′-fluoro-4′-(1,1,1,3,3,3-hexafluoro-2-hydroxy-2-yl)-[1,1′-biphenyl]-4-yl)-1-(2-nitrophenyl) methyl sulfonamide, N-(2′-fluoro-4′-(1,1,1,3,3,3-hexafluoro-2-hydroxy-2-yl)-[1,1′-biphenyl]-4-yl)-1-(4-nitrophenyl) methyl sulfonamide, 4-((N-(2′-fluoro-4′-(1,1,1,3,3,3-hexafluoro-2-hydroxy-2-yl)-[1,1′-biphenyl]4-yl) amino) methyl) benzoate, 3-((N-(2′-fluoro-4′-(1,1,1,3,3,3-hexafluoro-2-hydroxy-2-yl)-[1,1′-biphenyl] 4-yl) sulfamoyl) methyl) benzoic acid 2-((N-(2′-fluoro-4′-(1,1,1,3,3,3-hexafluoro-2-hydroxy-2-yl)-[1,1′-biphenyl] 4-yl) sulfamoyl) methyl) benzoic acid, 4-((N-(2′-fluoro-4′-(1,1,1,3,3,3-hexafluoro-2-hydroxy-2-yl)-[1,1′-biphenyl] 4-yl) sulfamoyl) methyl)-N-methylbenzamide, 4-((N-(2′-fluoro-4′-(1,1,1,3,3,3-hexafluoro-2-hydroxy-2-yl)-[1,1′-biphenyl] 4-yl) sulfamoyl) methyl) benzamide, 4-((N-(3′-fluoro-[1,1′-biphenyl]-4-yl) sulfamoyl) methyl) benzoate, 4-((N-(4′-fluoro-[1,1′-biphenyl]-4-yl) sulfamoyl) methyl) benzoate, 4-((N-(4′-chloro-biphenyl-4-yl) sulfamoyl) methyl) benzoate, 4-((N-(3′-chloro [1,1′-biphenyl]-4-yl) sulfamoyl) methyl) benzoate, 4-((N-(3′, 4′-difluoro [1,1′-biphenyl]-4-yl) sulfamoyl) methyl) benzoate, 4-((N-(2′-(trifluoromethoxy)-[1,1′-biphenyl]-4-yl) sulfamoyl) methyl) benzoate, 4-((N-(3′-methoxy [1,1′-biphenyl]-4-yl) sulfamoyl) methyl) benzoate, 4-((N-(4′-methoxy-[1,1′-biphenyl]-4-yl) sulfamoyl) methyl) benzoate, 4-((N-(2′-fluoro-[1,1′-biphenyl]-4-yl) sulfamoyl) methyl) benzoate, N-(2′-fluoro-4′-(1,1,1,3,3,3-hexafluoro-2-hydroxy-2-yl)-[1,1′-biphenyl]-4-yl) benzenesulfonamide, N-(2′-fluoro-4′-(1,1,1,3,3,3-hexafluoro-2-hydroxy-2-yl)-[1,1′-biphenyl]-4-yl)-2,3-dihydro-benzo [b] [1,4] dioxin-6-carboxamide, N-(2′-fluoro-4′-(1,1,1,3,3,3-hexafluoro-2-hydroxy-2-yl)-[1,1′-biphenyl]-4-yl)-4-phenoxy-benzamide, 4-((2′-fluoro-4′-(1,1,1,3,3,3-hexafluoro-2-hydroxy-2-yl)-[1,1′-biphenyl]-4-yl) carbamoyl) benzoate, N-(2′-fluoro-4′-(1,1,1,3,3,3-hexafluoro-2-hydroxy-2-yl)-[1,1′-biphenyl]-4-yl)-3-phenyl-propanamide, 4,4,4-trifluoro-N-(2′-fluoro-4′-(1,1,1,3,3,3-hexafluoro-2-hydroxy-2-yl)-[1,1′-biphenyl]-4-yl) butanamide, N-(2′-fluoro-4′-(1,1,1,3,3,3-hexafluoro-2-hydroxy-2-yl)-[1,1′-biphenyl]-4-yl) heptanamide, 4-cyclohexyl-N-(2′-fluoro-4′-(1,1,1,3,3,3-hexafluoro-2-hydroxy-2-yl)-[1,1′-biphenyl]-4-yl) butanamide, Methyl-4-((2′-fluoro-4′-(1,1,1,3,3,3-hexafluoro-2-hydroxy-2-yl)-[1,1′-biphenyl] 4-yl) amino)-4-oxobutanoic acid, Methyl-5-((2′-fluoro-4′-(1,1,1,3,3,3-hexafluoro-2-hydroxy-2-yl)-[1,1′-biphenyl] 4-yl) amino)-5-oxo-pentanoic acid, Methyl-6-((2′-fluoro-4′-(1,1,1,3,3,3-hexafluoro-2-hydroxy-2-yl)-[1,1′-biphenyl] 4-yl) amino)-6-oxo-hexanoate, N-(2′-fluoro-4′-(1,1,1,3,3,3-hexafluoro-2-hydroxy-2-yl)-[1,1′-biphenyl]-4-yl)-5-oxo-hexanamide, N-(2′-fluoro-4′-(1,1,1,3,3,3-hexafluoro-2-hydroxy-2-yl)-[1,1′-biphenyl]-4-yl)-1-(4-(trifluoromethyl) phenyl) methanesulfonamide, 4-((N-(2′-fluoro-4′-(1,1,1,3,3,3-hexafluoro-2-hydroxy-2-yl)-[1,1′-biphenyl] 4-yl) sulfamoyl) methyl) benzoate, and a combination thereof.

In some embodiments, the RORγ inhibitor compound of Formula I is a compound in Table 2.

TABLE 2
Exemplary Structures of RORγ inhibitor compounds of Formula I

| Structure | | |
|---|---|---|
| No. | Name | Structure |
| 1 | 1-ethyl-N-(2-fluoro-4-(1,1,1,3,3,3- hexafluoro-2-hydroxypropan-2- yl)phenyl)-2-oxo-1,2- dihydrobenzo[cd]indole-6-sulfonamide | 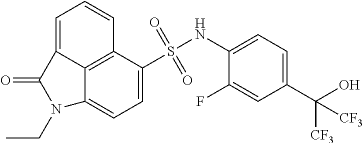 [SMILES: CCN1C(=O)c2cccc3c(S(=O)(=O)Nc4ccc(C(O)(C(F)(F)F)C(F)(F)F)cc4F)ccc1c23] |
| 2 | N-(2-fluoro-4-(1,1,1,3,3,3- hexafluoro-2-hydroxypropan-2- yl)phenyl)-4,4-dimethyl-2-oxo-1,2,3,4- tetrahydroquinoline-6-sulfonamide | 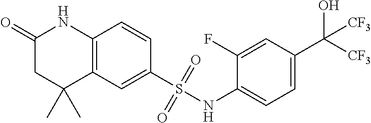 [SMILES: CC1(C)CC(=O)Nc2ccc(S(=O)(=O)Nc3ccc(C(O)(C(F)(F)F)C(F)(F)F)cc3F)cc21] |
| 3 | N-(2-fluoro-4-(1,1,1,3,3,3- hexafluoro-2-hydroxypropan-2- yl)phenyl)-2-oxo-1-propyl-1,2,3,4- tetrahydroquinoline-6-sulfonamide | 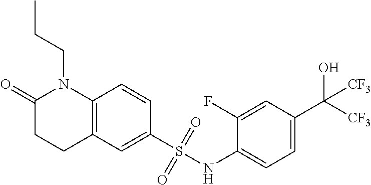 [SMILES: CCCN1C(=O)CCc2cc(S(=O)(=O)Nc3ccc(C(O)(C(F)(F)F)C(F)(F)F)cc3F)ccc21] |
| 4 | N-(2-fluoro-4-(1,1,1,3,3,3- hexafluoro-2-hydroxypropan-2- yl)phenyl)-2-oxo-3-propyl-2,3- dihydrobenzo[d]oxazole-6-sulfonamide | 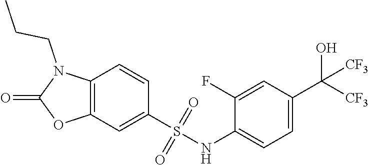 [SMILES: CCCn1c(=O)oc2cc(S(=O)(=O)Nc3ccc(C(O)(C(F)(F)F)C(F)(F)F)cc3F)ccc21] |
| 5 | N-(2-fluoro-4-(1,1,1,3,3,3- hexafluoro-2-hydroxypropan-2- yl)phenyl)-2-oxoindoline-5-sulfonamide | 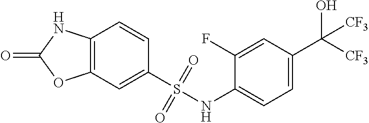 [SMILES: O=c1[nH]c2ccc(S(=O)(=O)Nc3ccc(C(O)(C(F)(F)F)C(F)(F)F)cc3F)cc2o1] |
| 6 | N-(2-fluoro-4-(1,1,1,3,3,3- hexafluoro-2-hydroxypropan-2- yl)phenyl)-2,4-dioxo-1,3-dipropyl- 1,2,3,4-tetrahydroquinazoline-6- sulfonamide | 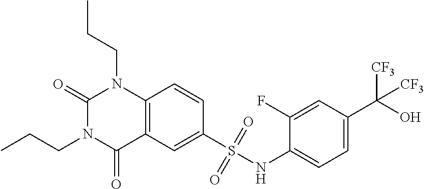 [SMILES: CCCn1c(=O)c2cc(S(=O)(=O)Nc3ccc(C(O)(C(F)(F)F)C(F)(F)F)cc3F)ccc2n(CCC)c1=O] |
| 7 | N-(2-fluoro-4-(1,1,1,3,3,3- hexafluoro-2-hydroxypropan-2- yl)phenyl)-2-oxo-1,2,3,4- tetrahydroquinoline-6-sulfonamide | 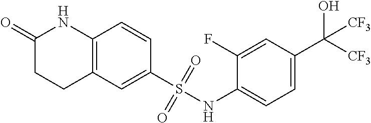 [SMILES: O=C1CCc2cc(S(=O)(=O)Nc3ccc(C(O)(C(F)(F)F)C(F)(F)F)cc3F)ccc2N1] |
| 8 | 1-ethyl-N-(2′-fluoro-4′-(1,1,1,3,3,3- hexafluoro-2-hydroxypropan-2-yl)-[1,1′- biphenyl]-4-yl)-2-oxo-1,2- dihydrobenzo[cd]indole-6-sulfonamide | 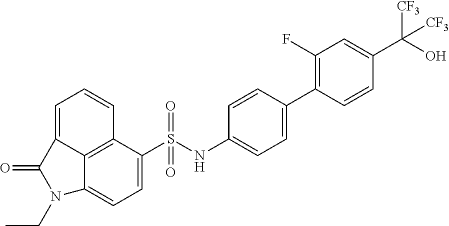 [SMILES: CCN1C(=O)c2cccc3c(S(=O)(=O)Nc4ccc(-c5ccc(C(O)(C(F)(F)F)C(F)(F)F)cc5F)cc4)ccc1c23] |
| 9 | N-(2′-fluoro-4′-(1,1,1,3,3,3- hexafluoro-2-hydroxypropan-2-yl)-[1,1′- biphenyl]-4-yl)-2-oxo-1-propyl-1,2,3,4- tetrahydroquinoline-6-sulfonamide | 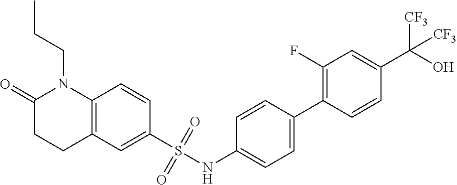 [SMILES: CCCN1C(=O)CCc2cc(S(=O)(=O)Nc3ccc(-c4ccc(C(O)(C(F)(F)F)C(F)(F)F)cc4F)cc3)ccc21] |
| 10 | N-(2′-fluoro-4′-(1,1,1,3,3,3- hexafluoro-2-hydroxypropan-2-yl)-[1,1′- biphenyl]-4-yl)-2-oxo-3-propyl-2,3- dihydrobenzo[d]oxazole-6-sulfonamide | 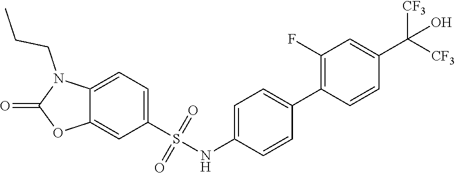 [SMILES: CCCn1c(=O)oc2cc(S(=O)(=O)Nc3ccc(-c4ccc(C(O)(C(F)(F)F)C(F)(F)F)cc4F)cc3)ccc21] |
| 11 | N-(2′-fluoro-4′-(1,1,1,3,3,3- hexafluoro-2-hydroxypropan-2-yl)-[1,1′- biphenyl]-4-yl)-2,4-dioxo-1,3-dipropyl- 1,2,3,4-tetrahydroquinazoline-6- sulfonamide | 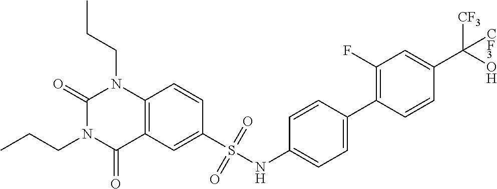 [SMILES: CCCn1c(=O)c2cc(S(=O)(=O)Nc3ccc(-c4ccc(C(O)(C(F)(F)F)C(F)(F)F)cc4F)cc3)ccc2n(CCC)c1=O] |
| 12 | N-(2′-fluoro-4′-(1,1,1,3,3,3- hexafluoro-2-hydroxypropan-2-yl)-[1,1′- biphenyl]-4-yl)-4,4-dimethyl-2-oxo- 1,2,3,4-tetrahydroquinoline-6- sulfonamide | 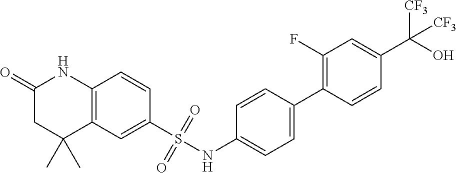 [SMILES: CC1(C)CC(=O)Nc2ccc(S(=O)(=O)Nc3ccc(-c4ccc(C(O)(C(F)(F)F)C(F)(F)F)cc4F)cc3)cc21] |
| 13 | N-(2′-fluoro-4′-(1,1,1,3,3,3- hexafluoro-2-hydroxypropan-2-yl)-[1,1′- biphenyl]-4-yl)-2-oxoindoline-5- sulfonamide | 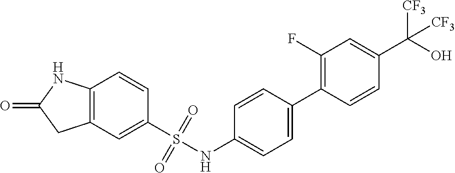 [SMILES: O=C1Cc2cc(S(=O)(=O)Nc3ccc(-c4ccc(C(O)(C(F)(F)F)C(F)(F)F)cc4F)cc3)ccc2N1] |
| 14 | N-(2′-fluoro-4′-(1,1,1,3,3,3- hexafluoro-2-hydroxypropan-2-yl)-[1,1′- biphenyl]-4-yl)-2,4-dioxo-1,2,3,4- tetrahydroquinazoline-6-sulfonamide | 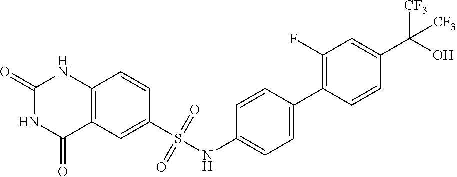 [SMILES: O=c1[nH]c(=O)c2cc(S(=O)(=O)Nc3ccc(-c4ccc(C(O)(C(F)(F)F)C(F)(F)F)cc4F)cc3)ccc2[nH]1] |
| 15 | N-(2′-fluoro-4′-(1,1,1,3,3,3- hexafluoro-2-hydroxypropan-2-yl)-[1,1′- biphenyl]-4-yl)-2,3- dihydrobenzo[d]oxazole-6-sulfonamide | 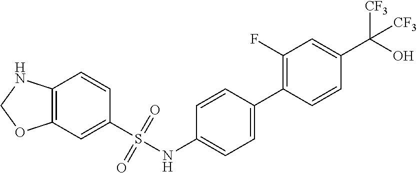 [SMILES: O=S(=O)(Nc1ccc(-c2ccc(C(O)(C(F)(F)F)C(F)(F)F)cc2F)cc1)c1ccc2c(c1)OCN2] |
| 16 | N-(2′-fluoro-4′-(1,1,1,3,3,3- hexafluoro-2-hydroxypropan-2-yl)-[1,1′- biphenyl]-4-yl)-2-oxo-1,2,3,4- tetrahydroquinoline-6-sulfonamide | 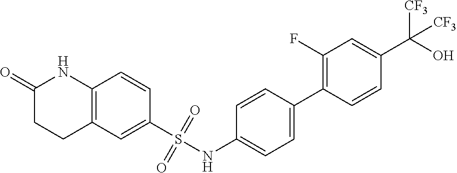 [SMILES: O=C1CCc2cc(S(=O)(=O)Nc3ccc(-c4ccc(C(O)(C(F)(F)F)C(F)(F)F)cc4F)cc3)ccc2N1] |
| 17 | N-(2′-fluoro-4′-(1,1,1,3,3,3- hexafluoro-2-hydroxypropan-2-yl)-[1,1′- biphenyl]-4-yl)-3-methyl-2-oxo-1,2,3,4- tetrahydroquinazoline-6-sulfonamide | 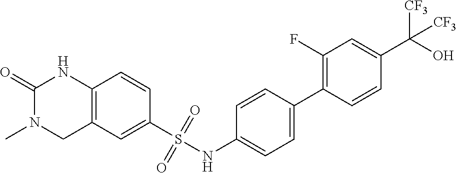 [SMILES: CN1Cc2cc(S(=O)(=O)Nc3ccc(-c4ccc(C(O)(C(F)(F)F)C(F)(F)F)cc4F)cc3)ccc2NC1=O] |
| 18 | 1-acetyl-N-(2′-fluoro-4′-(1,1,1,3,3,3- hexafluoro-2-hydroxypropan-2-yl)-[1,1′- biphenyl]-4-yl)indoline-5-sulfonamide | 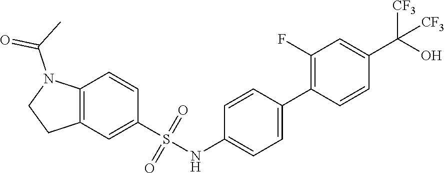 [SMILES: CC(=O)N1CCc2cc(S(=O)(=O)Nc3ccc(-c4ccc(C(O)(C(F)(F)F)C(F)(F)F)cc4F)cc3)ccc21] |
| 19 | N-(2′-fluoro-4′-(1,1,1,3,3,3- hexafluoro-2-hydroxypropan-2-yl)-[1,1′- biphenyl]-4-yl)-2,2-dimethyl-3-oxo-3,4- dihydro-2H-benzo[b][1,4]oxazine-6- sulfonamide | 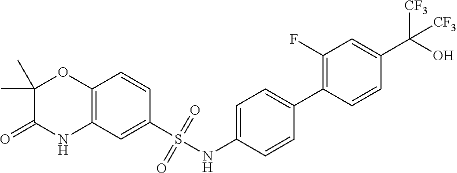 [SMILES: CC1(C)Oc2ccc(S(=O)(=O)Nc3ccc(-c4ccc(C(O)(C(F)(F)F)C(F)(F)F)cc4F)cc3)cc2NC1=O] |
| 20 | 4-acetyl-N-(2′-fluoro-4′-(1,1,1,3,3,3- hexafluoro-2-hydroxypropan-2-yl)-[1,1′- biphenyl]-4-yl)-2,2-dimethyl-3,4- dihydro-2H-benzo[b][1,4]oxazine-6- sulfonamide | 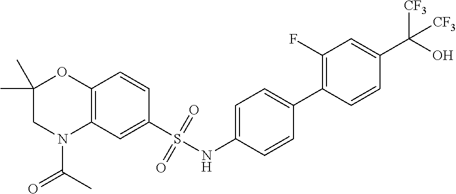 [SMILES: CC(=O)N1CC(C)(C)Oc2ccc(S(=O)(=O)Nc3ccc(-c4ccc(C(O)(C(F)(F)F)C(F)(F)F)cc4F)cc3)cc21] |
| 21 | N-(2′-fluoro-4′-(1,1,1,3,3,3- hexafluoro-2-hydroxypropan-2-yl)-[1,1′- biphenyl]-4-yl)-3-oxo-3,4-dihydro-2H- benzo[b][1,4]oxazine-6-sulfonamide | 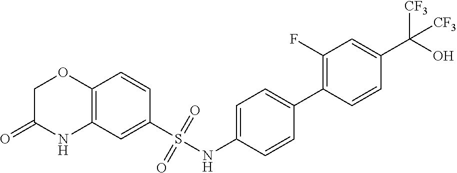 [SMILES: O=C1COc2ccc(S(=O)(=O)Nc3ccc(-c4ccc(C(O)(C(F)(F)F)C(F)(F)F)cc4F)cc3)cc2N1] |
| 22 | N-(2′-fluoro-4′-(1,1,1,3,3,3- hexafluoro-2-hydroxypropan-2-yl)-[1,1′- biphenyl]-4-yl)-2-oxo-1,4-dihydro-2H- benzo[d][1,3]oxazine-7-sulfonamide | 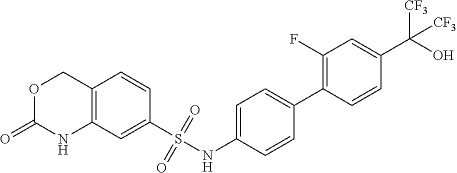 [SMILES: O=C1Nc2cc(S(=O)(=O)Nc3ccc(-c4ccc(C(O)(C(F)(F)F)C(F)(F)F)cc4F)cc3)ccc2CO1] |
| 23 | N-(2′-fluoro-4′-(1,1,1,3,3,3- hexafluoro-2-hydroxypropan-2-yl)-[1,1′- biphenyl]-4-yl)-4-methyl-3-oxo-3,4- dihydro-2H-benzo[b][1,4]oxazine-6- sulfonamide | 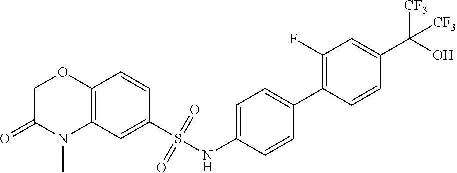 [SMILES: CN1C(=O)COc2ccc(S(=O)(=O)Nc3ccc(-c4ccc(C(O)(C(F)(F)F)C(F)(F)F)cc4F)cc3)cc21] |
| 24 | 4-acetyl-N-(2′-fluoro-4′-(1,1,1,3,3,3- hexafluoro-2-hydroxypropan-2-yl)-[1,1′- biphenyl]-4-yl)-3,4-dihydro-2H- benzo[b][1,4]oxazine-6-sulfonamide | 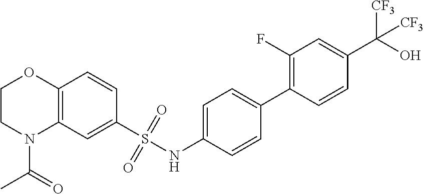 [SMILES: CC(=O)N1CCOc2ccc(S(=O)(=O)Nc3ccc(-c4ccc(C(O)(C(F)(F)F)C(F)(F)F)cc4F)cc3)cc21] |
| 25 | 4-fluoro-N-(2′-fluoro-4′-(1,1,1,3,3,3- hexafluoro-2-hydroxypropan-2-yl)-[1,1′- biphenyl]-4-yl)benzenesulfonamide | 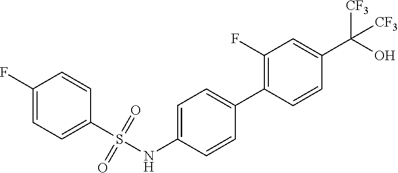 [SMILES: O=S(=O)(Nc1ccc(-c2ccc(C(O)(C(F)(F)F)C(F)(F)F)cc2F)cc1)c1ccc(F)cc1] |
| 26 | 2,4-difluoro-N-(2′-fluoro-4′- (1,1,1,3,3,3-hexafluoro-2-hydroxypropan- 2-yl)-[1,1′-biphenyl]-4- l)benzenesulfonamide | 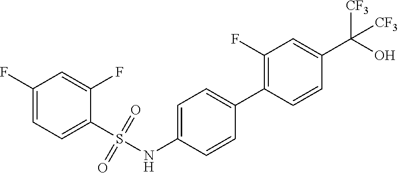 [SMILES: O=S(=O)(Nc1ccc(-c2ccc(C(O)(C(F)(F)F)C(F)(F)F)cc2F)cc1)c1ccc(F)cc1F] |
| 27 | N-(2′-fluoro-4′-(1,1,1,3,3,3- hexafluoro-2-hydroxypropan-2-yl)-[1,1′- biphenyl]-4-yl)thiophene-2-sulfonamide | 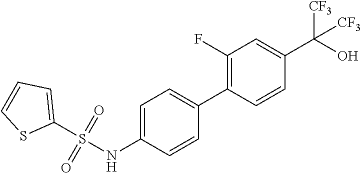 [SMILES: O=S(=O)(Nc1ccc(-c2ccc(C(O)(C(F)(F)F)C(F)(F)F)cc2F)cc1)c1cccs1] |
| 28 | N-(2′-fluoro-4′-(1,1,1,3,3,3- hexafluoro-2-hydroxypropan-2-yl)-[1,1′- biphenyl]-4-yl)-2,3- dihydrobenzo[b][1,4]dioxine-6- sulfonamide | 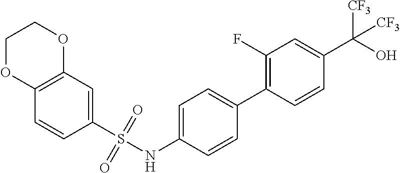 [SMILES: O=S(=O)(Nc1ccc(-c2ccc(C(O)(C(F)(F)F)C(F)(F)F)cc2F)cc1)c1ccc2c(c1)OCCO2] |
| 29 | N-(2′-fluoro-4′-(1,1,1,3,3,3- hexafluoro-2-hydroxypropan-2-yl)-[1,1′- biphenyl]-4-yl)-4- morpholinobenzenesulfonamide | 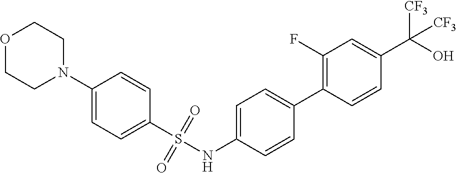 [SMILES: O=S(=O)(Nc1ccc(-c2ccc(C(O)(C(F)(F)F)C(F)(F)F)cc2F)cc1)c1ccc(N2CCOCC2)cc1] |
| 30 | N-(2′-fluoro-4′-(1,1,1,3,3,3- hexafluoro-2-hydroxypropan-2-yl)-[1,1′- biphenyl]-4-yl)-4- phenoxybenzenesulfonamide | 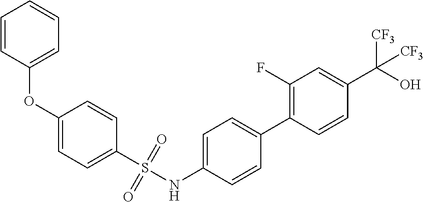 [SMILES: O=S(=O)(Nc1ccc(-c2ccc(C(O)(C(F)(F)F)C(F)(F)F)cc2F)cc1)c1ccc(Oc2ccccc2)cc1] |
| 31 | N-(2′-fluoro-4′-(1,1,1,3,3,3- hexafluoro-2-hydroxypropan-2-yl)-[1,1′- biphenyl]-4-yl)-2-(p-tolyl)acetamide | 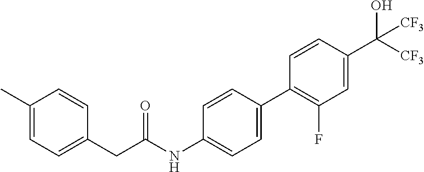 [SMILES: Cc1ccc(CC(=O)Nc2ccc(-c3ccc(C(O)(C(F)(F)F)C(F)(F)F)cc3F)cc2)cc1] |
| 32 | 2-(3,4-dimethoxyphenyl)-N-(2 ′- fluoro-4′-(1,1,1,3,3,3-hexafluoro-2- hydroxypropan-2-yl)-[1,1′-biphenyl]-4- yl)acetamide | 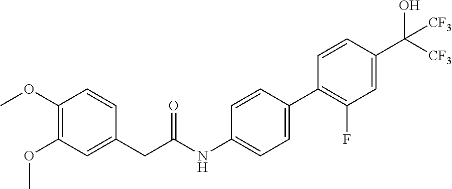 [SMILES: COc1ccc(CC(=O)Nc2ccc(-c3ccc(C(O)(C(F)(F)F)C(F)(F)F)cc3F)cc2)cc1OC] |
| 33 | 2-(4-chlorophenyl)-N-(2′-fluoro-4′- (1,1,1,3,3,3-hexafluoro-2-hydroxypropan- 2-yl)-[1,1′-biphenyl]-4-yl)acetamide | 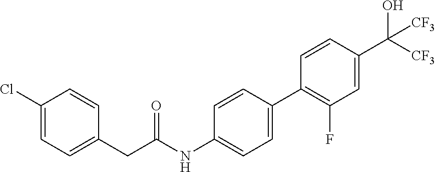 [SMILES: O=C(Cc1ccc(Cl)cc1)Nc1ccc(-c2ccc(C(O)(C(F)(F)F)C(F)(F)F)cc2F)cc1] |
| 34 | 2-(4-bromophenyl)-N-(2′-fluoro-4′- (1,1,1,3,3,3 -hexafluoro-2-hydroxypropan- 2-yl)-[1,1′-biphenyl]-4-yl)acetamide | 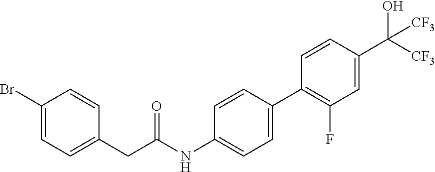 [SMILES: O=C(Cc1ccc(Br)cc1)Nc1ccc(-c2ccc(C(O)(C(F)(F)F)C(F)(F)F)cc2F)cc1] |
| 35 | 2-(3-chlorophenyl)-N-(2′-fluoro-4′- (1,1,1,3,3,3 -hexafluoro-2-hydroxypropan- 2-yl)-[1,1′-biphenyl]-4-yl)acetamide | 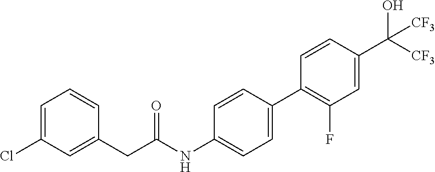 [SMILES: O=C(Cc1cccc(Cl)c1)Nc1ccc(-c2ccc(C(O)(C(F)(F)F)C(F)(F)F)cc2F)cc1] |
| 36 (F17) | N-(2′-fluoro-4′-(1,1,1,3,3,3- hexafluoro-2-hydroxypropan-2-yl)-[1,1′- biphenyl]-4-yl)-2-(4- nitrophenyl)acetamide | 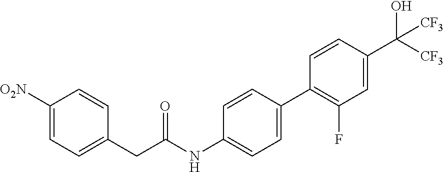 [SMILES: O=C(Cc1ccc([N+](=O)[O-])cc1)Nc1ccc(-c2ccc(C(O)(C(F)(F)F)C(F)(F)F)cc2F)cc1] |
| 37 (F18, XY018) | N-(2′-fluoro-4′-(1,1,1,3,3,3- hexafluoro-2-hydroxypropan-2-yl)-[1,1′- biphenyl]-4-yl)-2-(2- nitrophenyl)acetamide | 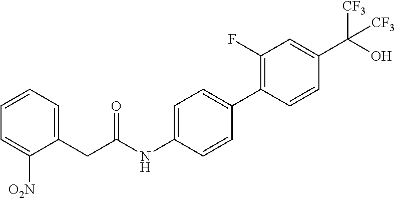 [SMILES: O=C(Cc1ccccc1[N+](=O)[O-])Nc1ccc(-c2ccc(C(O)(C(F)(F)F)C(F)(F)F)cc2F)cc1] |
| 38 | N-(2′-fluoro-4 ′-(1,1,1,3,3,3- hexafluoro-2-hydroxypropan-2-yl)-[1,1′- biphenyl]-4-yl)-2-(4- (trifluoromethyl)phenyl)acetamide | 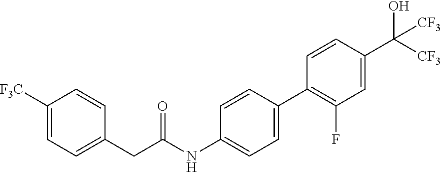 [SMILES: O=C(Cc1ccc(C(F)(F)F)cc1)Nc1ccc(-c2ccc(C(O)(C(F)(F)F)C(F)(F)F)cc2F)cc1] |
| 39 | N-(2′-fluoro-4′-(1,1,1,3,3,3- hexafluoro-2-hydroxypropan-2-yl)-[1,1′- biphenyl]-4-yl)pentanamide | 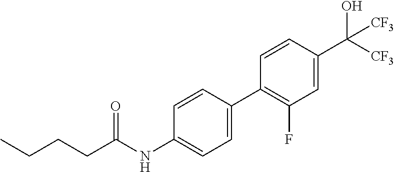 [SMILES: CCCCC(=O)Nc1ccc(-c2ccc(C(O)(C(F)(F)F)C(F)(F)F)cc2F)cc1] |
| 40 | N-(2′-fluoro-4′-(1,1,1,3,3,3- hexafluoro-2-hydroxypropan-2-yl)-[1,1′- biphenyl]-4-yl)-3,3-dimethylbutanamide | 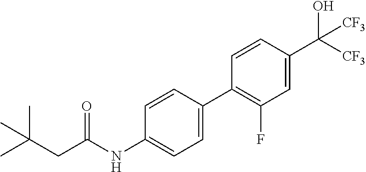 [SMILES: CC(C)(C)CC(=O)Nc1ccc(-c2ccc(C(O)(C(F)(F)F)C(F)(F)F)cc2F)cc1] |
| 41 | 2-(3,4-dichlorophenyl)-N-(2′-fluoro- 4′-(1,1,1,3,3,3-hexafluoro-2- hydroxypropan-2-yl)-[1,1′-biphenyl]-4- yl)acetamide | 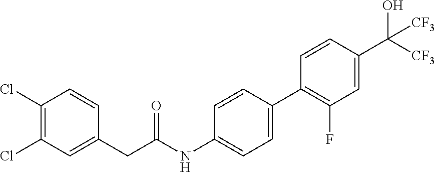 [SMILES: O=C(Cc1ccc(Cl)c(Cl)c1)Nc1ccc(-c2ccc(C(O)(C(F)(F)F)C(F)(F)F)cc2F)cc1] |
| 42 | N-(2′-fluoro-4′-(1,1,1,3,3,3- hexafluoro-2-hydroxypropan-2-yl)-[1,1′- biphenyl]-4-yl)benzamide | 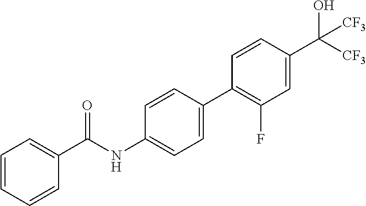 [SMILES: O=C(Nc1ccc(-c2ccc(C(O)(C(F)(F)F)C(F)(F)F)cc2F)cc1)c1ccccc1] |
| 43 | N-(2′-fluoro-4′-(1,1,1,3,3,3- hexafluoro-2-hydroxypropan-2-yl)-[1,1′- biphenyl]-4-yl)-1-(p- tolyl)methanesulfonamide | 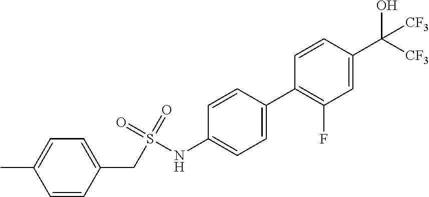 [SMILES: Cc1ccc(CS(=O)(=O)Nc2ccc(-c3ccc(C(O)(C(F)(F)F)C(F)(F)F)cc3F)cc2)cc1] |
| 44 | Methyl-4-((N-(2-fluoro-4-(1,1,1,3,3,3- hexafluoro-2-hydroxypropan-2- yl)phenyl)sulfamoyl)methyl)benzoate | 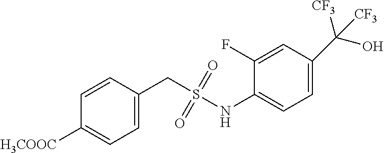 |
| 45 | 4-((N-(2′-fluoro-4′-(1,1,1,3,3,3- hexafluoro-2-hydroxypropan-2-yl)-[1,1′- biphenyl]-4-yl)sulfamoyl)methyl)benzoic acid | 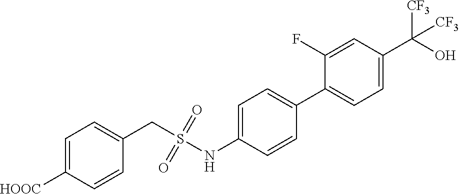 [SMILES: O=C(O)c1ccc(CS(=O)(=O)Nc2ccc(-c3ccc(C(O)(C(F)(F)F)C(F)(F)F)cc3F)cc2)cc1] |
| 46 | 4-((N-(2-fluoro-4-(1,1,1,3,3,3- hexafluoro-2-hydroxypropan-2- yl)phenyl)sulfamoyl)methyl)benzoic acid | 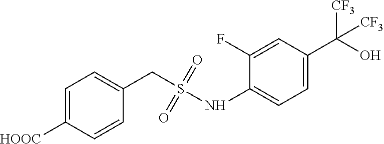 [SMILES: O=C(O)c1ccc(CS(=O)(=O)Nc2ccc(C(O)(C(F)(F)F)C(F)(F)F)cc2F)cc1] |
| 47 | Methyl-4-((N-(2′-fluoro-4′-(1,1,1,3,3,3- hexafluoro-2-hydroxypropan-2-yl)-[1,1′- biphenyl]-4-yl)-N- propylsulfamoyl)methyl)benzoate | 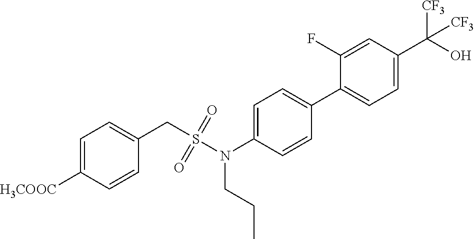 |
| 48 | methyl 4-((N-([1,1′-biphenyl]-4- yl)sulfamoyl)methyl)benzoate | 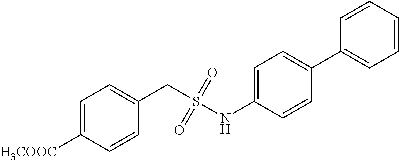 |
| 49 | methyl -4-((N-(4- acetylphenyl)sulfamoyl)methyl)benzoate | 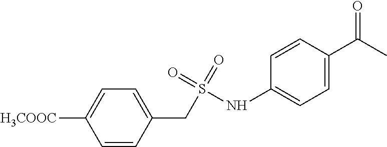 |
| 50 | methyl-4-((N-(3,4- dimethoxyphenyl)sulfamoyl)methyl) benzoate | 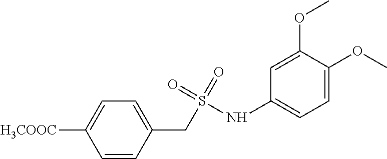 |
| 51 | N-(2′-fluoro-4′-(1,1,1,3,3,3- hexafluoro-2-hydroxypropan-2-yl)-[1,1′- biphenyl]-4-yl)-1-(4- (trifluoromethyl)phenyl) methanesulfonamide | 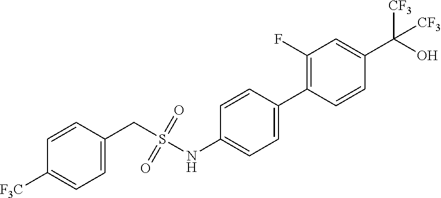 [SMILES: O=S(=O)(Cc1ccc(C(F)(F)F)cc1)Nc1ccc(-c2ccc(C(O)(C(F)(F)F)C(F)(F)F)cc2F)cc1] |
| 52 | Methyl-4-((N-(2′-fluoro-4′-(1,1,1,3,3,3- hexafluoro-2-hydroxypropan-2-yl)-[1,1′- biphenyl]-4- yl)sulfamoyl)methyl)benzoate | 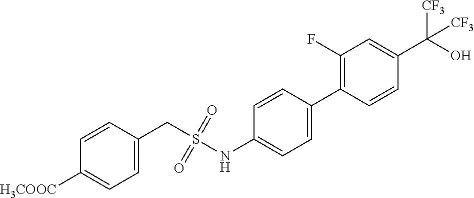 |
| 53 | 4-((N-(2-oxo-1-propyl-1,2,3,4- tetrahydroquinolin-6- yl)sulfamoyl)methyl)benzoic acid | 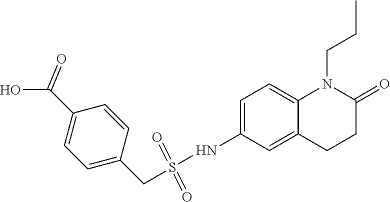 [SMILES: CCCN1C(=O)CCc2cc(NS(=O)(=O)Cc3ccc(C(=O)O)cc3)ccc21] |
| 54 | N-(2′-fluoro-4′-(1,1,1,3,3,3-hexafluoro-2- hydroxypropan-2-yl)-[1,1′-biphenyl]-4- yl)-1-(4- (methylsulfonyl)phenyl) methanesulfonamide | 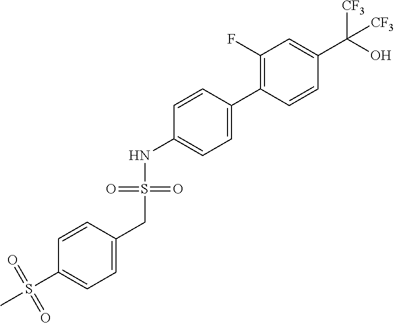 [SMILES: CS(=O)(=O)c1ccc(CS(=O)(=O)Nc2ccc(-c3ccc(C(O)(C(F)(F)F)C(F)(F)F)cc3F)cc2)cc1] |
| 55 | N-(2′-fluoro-4′-(1,1,1,3,3,3- hexafluoro-2-hydroxypropan-2-yl)-[1,1′- biphenyl]-4-yl)-4- (trifluoromethyl)benzamide | 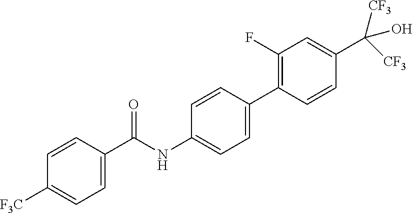 [SMILES: O=C(Nc1ccc(-c2ccc(C(O)(C(F)(F)F)C(F)(F)F)cc2F)cc1)c1ccc(C(F)(F)F)cc1] |
| 56 | Methyl-2-((N-(2′-fluoro-4′-(1,1,1,3,3,3- hexafluoro-2-hydroxypropan-2-yl)-[1,1′- biphenyl]-4- yl)sulfamoyl)methyl)benzoate | 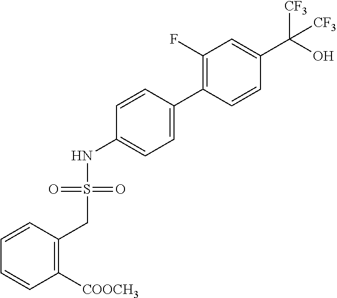 [SMILES: COC(=O)c1ccccc1CS(=O)(=O)Nc1ccc(-c2ccc(C(O)(C(F)(F)F)C(F)(F)F)cc2F)cc1] |
| 57 | N-(2′-fluoro-4′-(1,1,1,3,3,3-hexafluoro-2- hydroxypropan-2-yl)[1,1′-biphenyl]-4- yl)-1-(3-nitrophenyl)methanesulfonamide | 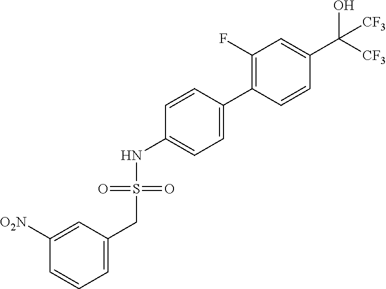 [SMILES: O=[N+]([O-])c1cccc(CS(=O)(=O)Nc2ccc(-c3ccc(C(O)(C(F)(F)F)C(F)(F)F)cc3F)cc2)c1] |
| 58 | Methyl-3-((N-(2′-fluoro-4′-(1,1,1,3,3,3- hexafluoro-2-hydroxypropan-2-yl)-[1,1′- biphenyl]-4- yl)sulfamoyl)methyl)benzoate | 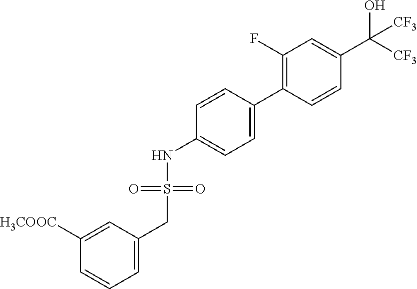 |
| 59 | 1-(4-cyanophenyl)-N-(2′-fluoro-4′- (1,1,1,3,3,3-hexafluoro-2-hydroxypropan- 2-yl)-[1,1′-biphenyl]-4- yl)methanesulfonamide | 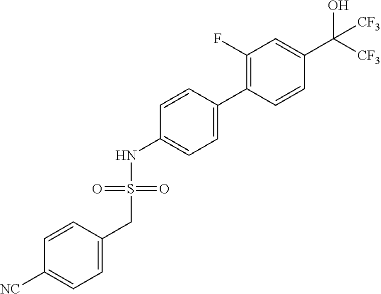 [SMILES: N#Cc1ccc(CS(=O)(=O)Nc2ccc(-c3ccc(C(O)(C(F)(F)F)C(F)(F)F)cc3F)cc2)cc1] |
| 60 | N-(2′-fluoro-4′-(1,1,1,3,3,3-hexafluoro-2- hydroxypropan-2-yl)-[1,1′-biphenyl]-4- yl)-1-(2-nitrophenyl)methanesulfonamide | 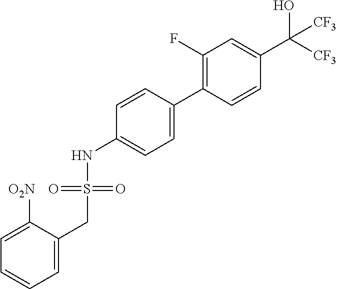 [SMILES: O=[N+]([O-])c1ccccc1CS(=O)(=O)Nc1ccc(-c2ccc(C(O)(C(F)(F)F)C(F)(F)F)cc2F)cc1] |
| 61 | N-(2′-fluoro-4′-(1,1,1,3,3,3- hexafluoro-2-hydroxypropan-2-yl)-[1,1′- biphenyl]-4-yl)-1-(4- nitrophenyl)methanesulfonamide | 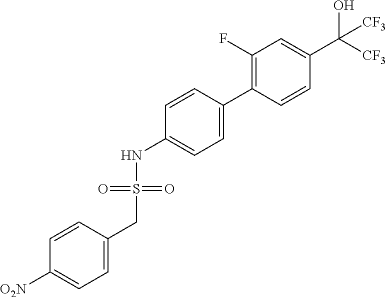 [SMILES: O=[N+]([O-])c1ccc(CS(=O)(=O)Nc2ccc(-c3ccc(C(O)(C(F)(F)F)C(F)(F)F)cc3F)cc2)cc1] |
| 62 | Ethyl-4-((N-(2′-fluoro-4′- (1,1,1,3,3,3-hexafluoro-2-hydroxypropan- 2-yl)[1,1′-biphenyl]-4- yl)sulfamoyl)methyl)benzoate | 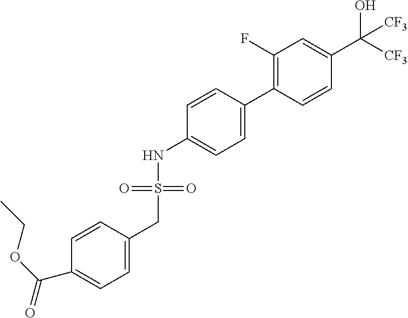 [SMILES: CCOC(=O)c1ccc(CS(=O)(=O)Nc2ccc(-c3ccc(C(O)(C(F)(F)F)C(F)(F)F)cc3F)cc2)cc1] |
| 63 | 3-((N-(2′-fluoro-4′-(1,1,1,3,3,3- hexafluoro-2-hydroxypropan-2-yl)-[1,1′- biphenyl]-4-yl)sulfamoyl)methyl)benzoic acid | 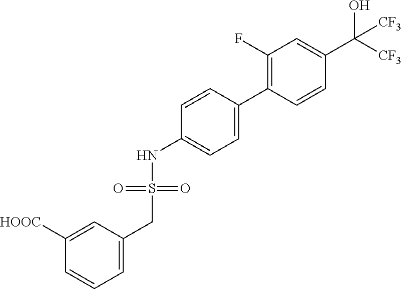 [SMILES: O=C(O)c1cccc(CS(=O)(=O)Nc2ccc(-c3ccc(C(O)(C(F)(F)F)C(F)(F)F)cc3F)cc2)c1] |
| 64 | 2-((N-(2′-fluoro-4′-(1,1,1,3,3,3- hexafluoro-2-hydroxypropan-2-yl)-[1,1′- biphenyl]-4-yl)sulfamoyl)methyl)benzoic acid | 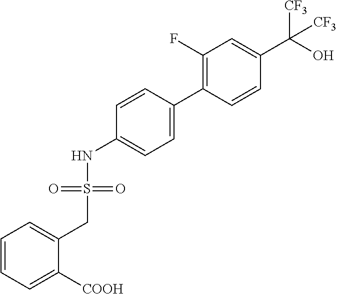 [SMILES: O=C(O)c1ccccc1CS(=O)(=O)Nc1ccc(-c2ccc(C(O)(C(F)(F)F)C(F)(F)F)cc2F)cc1] |
| 65 | 4-((N-(2′-fluoro-4′-(1,1,1,3,3,3- hexafluoro-2-hydroxypropan-2-yl)-[1,1′- biphenyl -4-yl)sulfamoyl)methyl)-N- methylbenzamide | 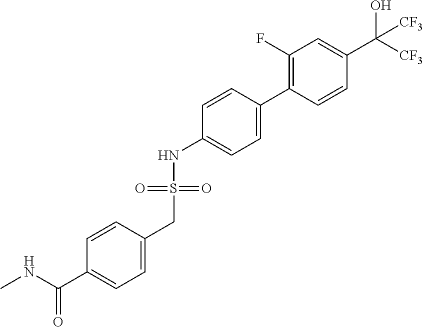 [SMILES: CNC(=O)c1ccc(CS(=O)(=O)Nc2ccc(-c3ccc(C(O)(C(F)(F)F)C(F)(F)F)cc3F)cc2)cc1] |
| 66 | 4-((N-(2′-fluoro-4′-(1,1,1,3,3,3- hexafluoro-2-hydroxypropan-2-yl)-[1,1′- biphenyl]-4- yl)sulfamoyl)methyl)benzamide | 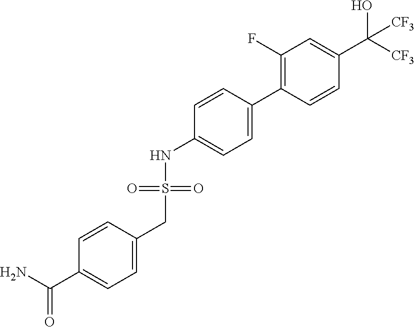 [SMILES: NC(=O)c1ccc(CS(=O)(=O)Nc2ccc(-c3ccc(C(O)(C(F)(F)F)C(F)(F)F)cc3F)cc2)cc1] |
| 67 | methyl -4-((N-(3′-fluoro-[1,1′- biphenyl]-4- yl)sulfamoyl)methyl)benzoate | 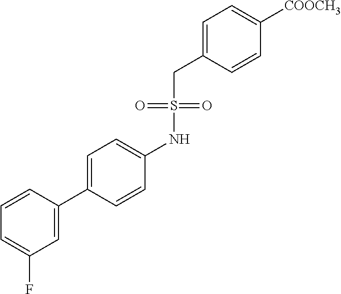 [SMILES: COC(=O)c1ccc(CS(=O)(=O)Nc2ccc(-c3cccc(F)c3)cc2)cc1] |
| 68 | methyl-4-((N-(4′-fluoro-[1,1′- biphenyl]-4- yl)sulfamoyl)methyl)benzoate | 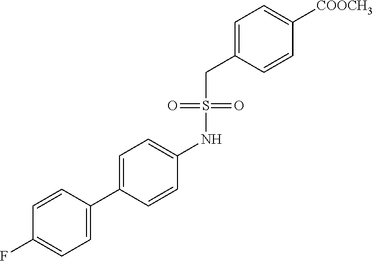 [SMILES: COC(=O)c1ccc(CS(=O)(=O)Nc2ccc(-c3ccc(F)cc3)cc2)cc1] |
| 69 | methyl -4-((N-(4′-chloro-[1,1′-biphenyl]- 4-yl)sulfamoyl)methyl)benzoate | 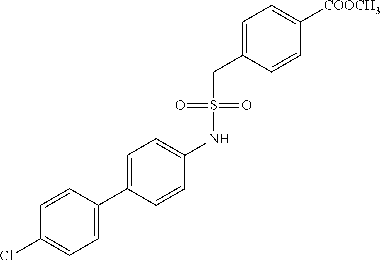 [SMILES: COC(=O)c1ccc(CS(=O)(=O)Nc2ccc(-c3ccc(Cl)cc3)cc2)cc1] |
| 70 | Methyl- 4-((N-(3′-chloro-[1,1′-biphenyl]- 4-yl)sulfamoyl)methyl)benzoate | 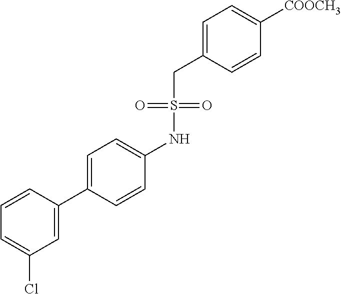 [SMILES: COC(=O)c1ccc(CS(=O)(=O)Nc2ccc(-c3cccc(Cl)c3)cc2)cc1] |
| 71 | methyl 4-((N-(3′,4′-difluoro-[1,1′- biphenyl]-4- yl)sulfamoyl)methyl)benzoate | 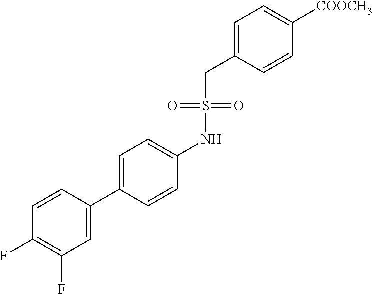 [SMILES: COC(=O)c1ccc(CS(=O)(=O)Nc2ccc(-c3ccc(F)c(F)c3)cc2)cc1] |
| 72 | Methyl-4-((N-(2′-(trifluoromethoxy)- [1,1′-biphenyl]-4- yl)sulfamoyl)methyl)benzoate | 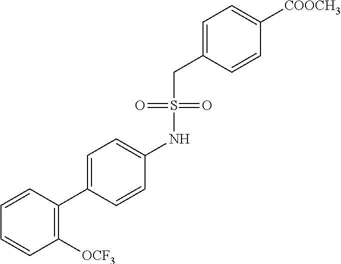 [SMILES: COC(=O)c1ccc(CS(=O)(=O)Nc2ccc(-c3ccccc3OC(F)(F)F)cc2)cc1] |
| 73 | methyl-4-((N-(3′-methoxy-[1,1′- biphenyl]-4- yl)sulfamoyl)methyl)benzoate | 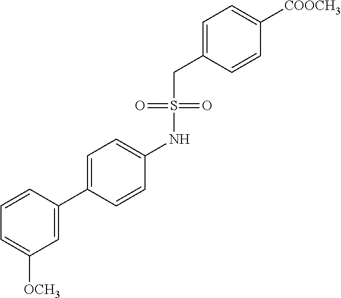 [SMILES: COC(=O)c1ccc(CS(=O)(=O)Nc2ccc(-c3cccc(OC)c3)cc2)cc1] |
| 74 | methyl -4-((N-(4′-methoxy-[1,1]- biphenyl]-4- yl)sulfamoyl)methyl)benzoate | 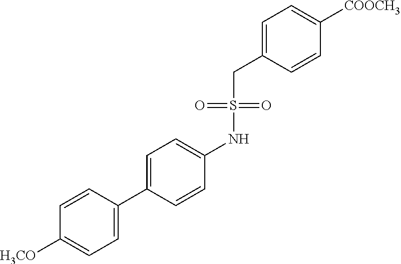 [SMILES: COC(=O)c1ccc(CS(=O)(=O)Nc2ccc(-c3ccc(OC)cc3)cc2)cc1] |
| 75 | methyl -4-((N-(2′-fluoro-[1, 1′- biphenyl]-4- yl)sulfamoyl)methyl)benzoate | 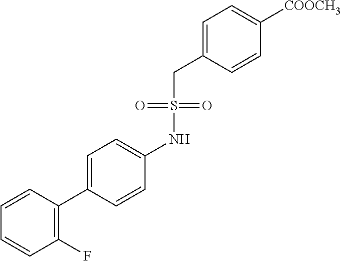 [SMILES: COC(=O)c1ccc(CS(=O)(=O)Nc2ccc(-c3ccccc3F)cc2)cc1] |
| 76 | N-(2′-fluoro-4′-(1,1,1,3,3,3-hexafluoro-2- hydroxypropan-2-yl)-[1,1′-biphenyl]-4- yl)benzenesulfonamide | 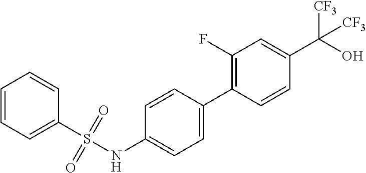 [SMILES: O=S(=O)(Nc1ccc(-c2ccc(C(O)(C(F)(F)F)C(F)(F)F)cc2F)cc1)c1ccccc1] |
| 77 | N-(2′-fluoro-4′-(1,1,1,3,3,3- hexafluoro-2-hydroxypropan-2-yl)-[1,1′- biphenyl]-4-yl)-2,3- dihydrobenzo[b][1,4]dioxine-6- carboxamide | 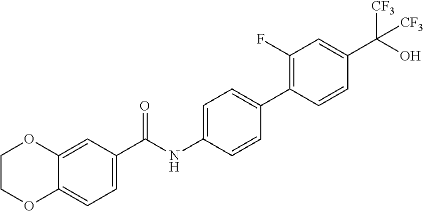 [SMILES: O=C(Nc1ccc(-c2ccc(C(O)(C(F)(F)F)C(F)(F)F)cc2F)cc1)c1ccc2c(c1)OCCO2] |
| 78 | N-(2′-fluoro-4′-(1,1,1,3,3,3- hexafluoro-2-hydroxypropan-2-yl)-[1,1′- biphenyl]-4-yl)-4-phenoxybenzamide | 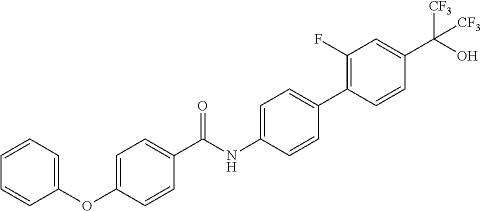 [SMILES: O=C(Nc1ccc(-c2ccc(C(O)(C(F)(F)F)C(F)(F)F)cc2F)cc1)c1ccc(Oc2ccccc2)cc1] |
| 79 | Methyl-4-((2′-fluoro-4′-(1,1,1,3,3,3- hexafluoro-2-hydroxypropan-2-yl)-[1,1′- biphenyl]-4-yl)carbamoyl)benzoate | 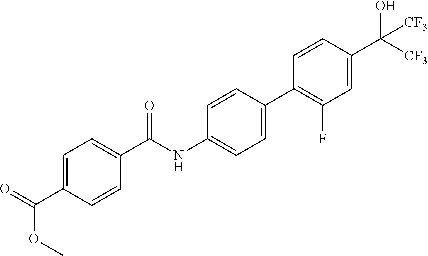 [SMILES: COC(=O)c1ccc(C(=O)Nc2ccc(-c3ccc(C(O)(C(F)(F)F)C(F)(F)F)cc3F)cc2)cc1] |
| 80 (F62) | N-(2′-fluoro-4′-(1,1,1,3,3,3-hexafluoro-2- hydroxypropan-2-yl)-[1,1′-biphenyl]-4- yl)-3-phenylpropanamide | 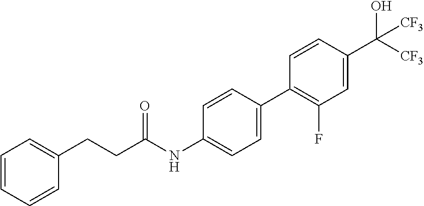 [SMILES: O=C(CCc1ccccc1)Nc1ccc(-c2ccc(C(O)(C(F)(F)F)C(F)(F)F)cc2F)cc1] |
| 81 (F63) | 4,4,4-trifluoro-N-(2′-fluoro-4′- (1,1,1,3,3,3 -hexafluoro-2-hydroxypropan- 2-yl)-[1,1′-biphenyl]-4-yl)butanamide | 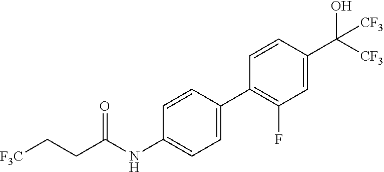 [SMILES: O=C(CCC(F)(F)F)Nc1ccc(-c2ccc(C(O)(C(F)(F)F)C(F)(F)F)cc2F)cc1] |
| 82 (F64) | N-(2′-fluoro-4′-(1,1,1,3,3,3-hexafluoro-2- hydroxypropan-2-yl)-[1,1′-biphenyl]-4- yl)heptanamide | 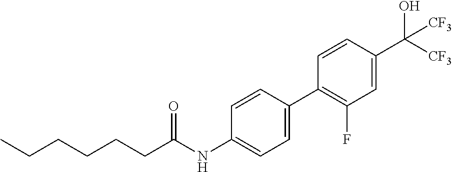 [SMILES: CCCCCCC(=O)Nc1ccc(-c2ccc(C(O)(C(F)(F)F)C(F)(F)F)cc2F)cc1] |
| 83 (F65) | 4-cyclohexyl-N-(2′-fluoro-4′- (1,1,1,3,3,3 -hexafluoro-2-hydroxypropan- 2-yl)-[1,1′-biphenyl]-4-yl)butanamide | 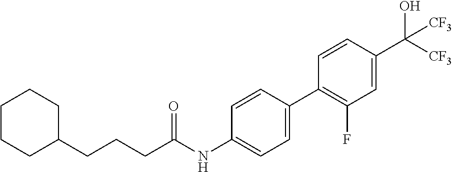 [SMILES: O=C(CCCC1CCCCC1)Nc1ccc(-c2ccc(C(O)(C(F)(F)F)C(F)(F)F)cc2F)cc1] |
| 84 | N-(2′-fluoro-4′-(1,1,1,3,3,3- hexafluoro-2-hydroxypropan-2-yl)-[1,1′- biphenyl]-4-yl)hexanamide | 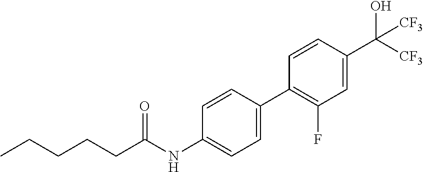 [SMILES: CCCCCC(=O)Nc1ccc(-c2ccc(C(O)(C(F)(F)F)C(F)(F)F)cc2F)cc1] |
| 85 | Methyl-4-((2′-fluoro-4′-(1,1,1,3,3,3- hexafluoro-2-hydroxypropan-2-yl)-[1,1′- biphenyl]-4-yl)amino)-4-oxobutanoate | 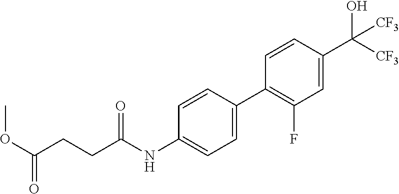 [SMILES: COC(=O)CCC(=O)Nc1ccc(-c2ccc(C(O)(C(F)(F)F)C(F)(F)F)cc2F)cc1] |
| 86 (F68) | Methyl-5-((2′-fluoro-4′-(1,1,1,3,3,3- hexafluoro-2-hydroxypropan-2-yl)-[1,1′- biphenyl]-4-yl)amino)-5-oxopentanoate | 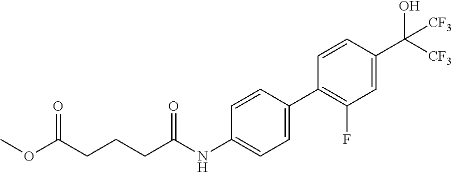 [SMILES: COC(=O)CCCC(=O)Nc1ccc(-c2ccc(C(O)(C(F)(F)F)C(F)(F)F)cc2F)cc1] |
| 87 | Methyl-6-((2′-fluoro-4′-(1,1,1,3,3,3- hexafluoro-2-hydroxypropan-2-yl)-[1,1′- biphenyl]-4-yl)amino)-6-oxohexanoate | 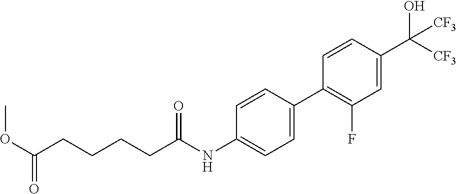 [SMILES: COC(=O)CCCCC(=O)Nc1ccc(-c2ccc(C(O)(C(F)(F)F)C(F)(F)F)cc2F)cc1] |
| 88 | N-(2′-fluoro-4′-(1,1,1,3,3,3-hexafluoro-2- hydroxypropan-2-yl)-[1,1′-biphenyl]-4- yl)-5-oxohexanamide | 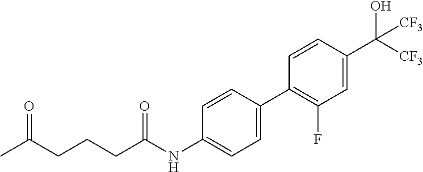 [SMILES: CC(=O)CCCC(=O)Nc1ccc(-c2ccc(C(O)(C(F)(F)F)C(F)(F)F)cc2F)cc1] |

In particular embodiments, the RORγ inhibitor compound of Formula I is represented by a compound according to any one of Formulas Ic to Ii:

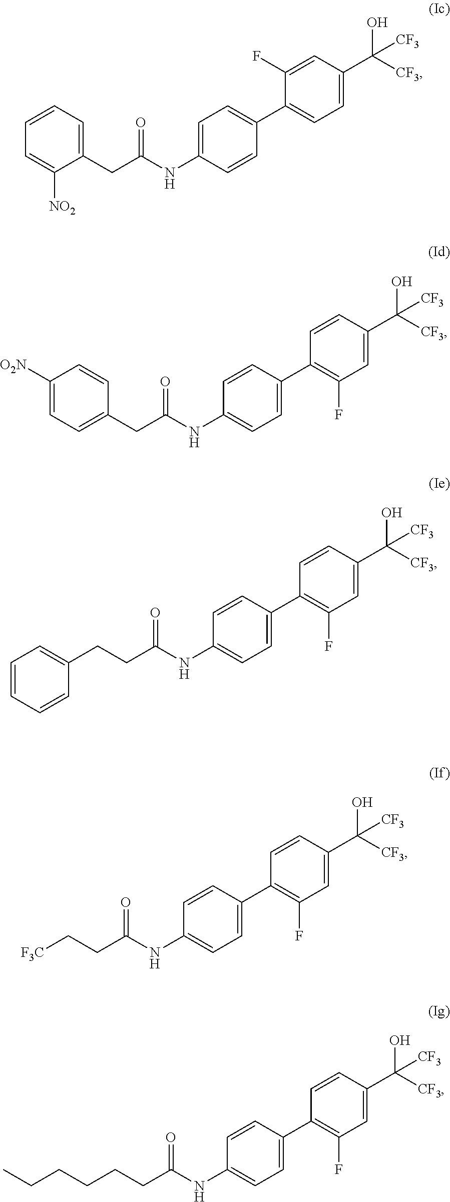 [SMILES: O=C(Cc1ccccc1[N+](=O)[O-])Nc1ccc(-c2ccc(C(O)(C(F)(F)F)C(F)(F)F)cc2F)cc1 ; O=C(Cc1ccc([N+](=O)[O-])cc1)Nc1ccc(-c2ccc(C(O)(C(F)(F)F)C(F)(F)F)cc2F)cc1 ; O=C(CCc1ccccc1)Nc1ccc(-c2ccc(C(O)(C(F)(F)F)C(F)(F)F)cc2F)cc1 ; O=C(CCC(F)(F)F)Nc1ccc(-c2ccc(C(O)(C(F)(F)F)C(F)(F)F)cc2F)cc1 ; CCCCCCC(=O)Nc1ccc(-c2ccc(C(O)(C(F)(F)F)C(F)(F)F)cc2F)cc1]

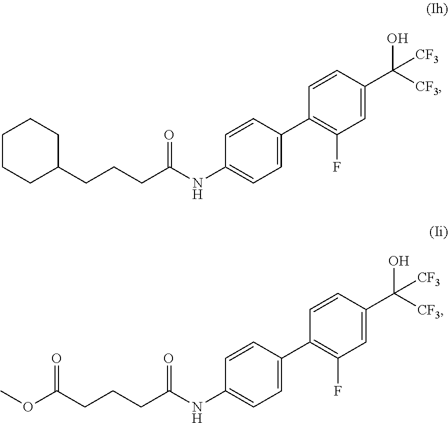 [SMILES: O=C(CCCC1CCCCC1)Nc1ccc(-c2ccc(C(O)(C(F)(F)F)C(F)(F)F)cc2F)cc1 ; COC(=O)CCCC(=O)Nc1ccc(-c2ccc(C(O)(C(F)(F)F)C(F)(F)F)cc2F)cc1]

or a pharmaceutically acceptable salt thereof, a derivative thereof, an analog thereof, or a combination thereof.

The compound of Formula Ic is also called XY018 or F18 and corresponds to Structure No. 37 in Table 2. The compound of Formula Id is also called F17 and corresponds to Structure No. 36 in Table 2. The compound of Formula Ie is also called F62 and corresponds to Structure No. 80 in Table 2. The compound of Formula If is also called F63 and corresponds to Structure No. 81 in Table 2. The compound of Formula Ig is also called F64 and corresponds to Structure No. 82 in Table 2. The compound of Formula Ih is also called F65 and corresponds to Structure No. 83 in Table 2. The compound of Formula Ii is also called F68 and corresponds to Structure No. 86 in Table 2.

In some embodiments, the RORγ inhibitor compound of Formula I is a compound disclosed in Chinese Patent Application No. 201410344302.0, filed Jul. 18, 2014, the disclosure of which is hereby incorporated by reference in its entirety for all purposes.

In some embodiments, the RORγ inhibitor compound of Formula I is a compound disclosed in Chinese Patent Publication No. CN 105272904, the disclosure of which is hereby incorporated by reference in its entirety for all purposes.

B. Anticancer Drugs

In certain embodiments, compounds of Formula I can be used in combination with an anticancer drug to reduce or reverse cancer cell resistance to the anticancer drug by sensitizing the cancer cell to the anticancer drug.

Non-limiting examples of anticancer drugs include anti-androgen drugs, chemotherapeutic agents, radiotherapeutic agents, antigen-specific immunotherapeutic agents, endocrine therapies, tyrosine kinase inhibitors, and combinations thereof.

1. Anti-Androgen Drugs

Anti-androgen drugs are compounds that inhibit the transcription, translation, stability, and/or activity of androgen receptors (AR) or variants thereof (e.g. AR-V7). Inhibition of AR activity can include inhibition of recruitment of AR to Androgen Response Elements (AREs). In some embodiments, inhibition of AR activity can include inhibition of recruitment of AR to the PSA promoter. In some embodiments, inhibition of AR activity can include inhibition of AR-induced activation of the PSA promoter. In some embodiments, inhibition of AR activity can include inhibition of AR-induced PSA production. For example, inhibition of AR can include inhibition of production of PSA in the absence of DHT.

Anti-androgen drugs include, but are not limited to, enzalutamide, abiraterone, bicalutamide, flutamide, nilutamide, apalutamide, finasteride, dutasteride, alfatradiol, and combinations thereof.

In some embodiments, the present invention provides a composition comprising one or more compounds of Formula I in combination with one or more anti-androgen drugs. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more anti-androgen drugs. In certain instances, the effective amount of one or more compounds of Formula I is an amount sufficient to sensitize an anti-androgen drug-resistant cancer such as anti-androgen drug-resistant prostate cancer (e.g., castration-resistant prostate cancer) to anti-androgen drug treatment. Compounds of Formula I and anti-androgen drugs can be delivered to a subject via the same route of administration (e.g., orally or parenterally) or via different routes of administration (e.g., intravenously for compounds of Formula I and orally for anti-androgen drugs, or vice versa).

2. Chemotherapeutic Agents

Chemotherapeutic agents are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol), docetaxel, cabazitaxel, hongdoushan A, hongdoushan B, hongdoushan C, baccatin I, baccatin I, and 10-deacetylbaccatin; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof, derivatives thereof, and combinations thereof.

In some embodiments, the present invention provides a composition comprising one or more compounds of Formula I in combination with one or more chemotherapeutic agents. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more chemotherapeutic agents. In certain instances, the effective amount of one or more compounds of Formula I is an amount sufficient to sensitize a chemotherapy drug-resistant cancer such as a tamoxifen-resistant cancer (e.g., tamoxifen-resistant breast cancer) or a taxane-resistant cancer (e.g., docetaxel-resistant prostate cancer) to chemotherapy drug treatment. Compounds of Formula I and chemotherapeutic agents can be delivered to a subject via the same route of administration (e.g., orally or parenterally) or via different routes of administration (e.g., intravenously for compounds of Formula I and orally for chemotherapeutic agents, or vice versa).

3. Radiotherapeutic Agents

Radiotherapeutic agents are well known in the art and can comprise external-beam radiation therapy and/or internal radiation therapy. External beam radiation therapy delivers radioactive beams of high energy X-rays and/or gamma rays to a patient's tumor, whereas internal radiation therapy delivers radioactive atoms to a patient's tumor. Both external beam radiation therapy and internal radiation therapy are used to suppress tumor growth or kill cancer cells by delivering a sufficient quantity of radioactivity to the target site. In some embodiments, the radiotherapeutic agent comprises a radioactive atom and is complexed with a biologic or synthetic agent to increase delivery to the target site. Such biologic or synthetic agents are known in the art. Suitable radioactive atoms for use with the compounds of Formula I include any of the radionuclides described herein, or any other isotope which emits enough energy to destroy a targeted tissue or cell. In some embodiments, radiotherapeutic agents may be coupled to targeting moieties, such as antibodies, to improve the localization of radiotherapeutic agents to cancerous cells.

The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the present invention include, but are not limited to, fluorine 18 (¹⁸F), fluorine 19 (¹⁹F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh), silver 111 (¹¹¹Ag), indium 111 (₁₁₁In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁶Re), thallium 201 (²⁰¹Tl), astatine 211 (²¹¹At), and bismuth 212 (²¹²Bi). As used herein, the “m” in ^{117m}Sn and ^{99m}Tc stands for the meta state. Additionally, naturally-occurring radioactive elements such as uranium, radium, and thorium, which typically represent mixtures of radioisotopes, are suitable examples of radionuclides. ⁶⁷Cu, ¹³¹I, ¹⁷⁷Lu, and ¹⁸⁶Re are beta- and gamma-emitting radionuclides. ²²¹Bi is an alpha- and beta-emitting radionuclide. ²¹¹At is an alpha-emitting radionuclide. ³²P, ⁴⁷Sc, ⁸⁹Sr, ⁹⁰Y, ¹⁵Rh, ¹¹¹Ag, ^{117m}Sn, ¹⁴⁹Pm, ¹⁵³Sm, ¹⁶⁶Ho, and ¹⁸⁶Re are examples of beta-emitting radionuclides. ⁶⁷Ga, ¹¹¹In, ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

In some embodiments, the present invention provides a composition comprising one or more compounds of Formula I in combination with one or more radiotherapeutic agents.

In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more radiotherapeutic agents. In certain instances, the effective amount of one or more compounds of Formula I is an amount sufficient to sensitize a radiation-resistant cancer such as a radiation-resistant breast cancer to radiation treatment. Compounds of Formula I and radiotherapeutic agents can be delivered to a subject via the same route of administration (e.g., orally or parenterally) or via different routes of administration (e.g., intravenously for compounds of Formula I and orally for radiotherapeutic agents, or vice versa).

4. Endocrine Therapies

Endocrine therapy is the manipulation of the endocrine system through the administration of specific hormones or drugs which inhibit or decrease the production or activity of targeted hormones or alter the gene expression pattern of targeted cells. Endocrine therapy is particularly useful in certain types of cancer, including breast cancer. Any known hormone antagonist or modulator may be used in the present invention. Endocrine therapies useful in the present invention include, but are not limited to, aromatase inhibitors (e.g. letrozole), megestrol acetate, flutamide, tamoxifen, raloxifene, lasofoxifene, bazedoxifene, bazedoxifene/conjugated estrogens, and combinations thereof.

In some embodiments, the present invention provides a composition comprising one or more compounds of Formula I in combination with one or more endocrine therapies. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more endocrine therapies. In certain instances, the effective amount of one or more compounds of Formula I is an amount sufficient to sensitize an endocrine therapy-resistant cancer such as a tamoxifen-resistant breast cancer to endocrine therapy. Compounds of Formula I and endocrine therapies can be delivered to a subject via the same route of administration (e.g., orally or parenterally) or via different routes of administration (e.g., intravenously for compounds of Formula I and orally for endocrine therapies, or vice versa).

5. Tyrosine Kinase Inhibitors

Tyrosine kinase inhibitors are small molecules that inhibit tyrosine kinase proteins. Tyrosine kinases are enzymes that activate many proteins in cellular signal transduction cascades by addition of a phosphate group to the protein. High expression and aberrant activation, of tyrosine kinase proteins can cause undesirable “switching on” of cellular signaling pathways that can result in uncontrolled cellular proliferation associated with cancerous cellular phenotypes. Various forms of cancer are currently treated by inhibiting or reducing the activity of poorly regulated tyrosine kinase proteins with tyrosine kinase inhibitors. Treatment regimens with tyrosine kinase inhibitors can suppress, reduce the incidence, reduce the severity, or inhibit the progression of cancer. Examples of tyrosine kinase inhibitors include, but are not limited to, gefitinib, erlotinib, sorafenib, sunitinib, dasatinib, lapatinib, nilotinib, bortezomib, salinomycin, and combinations thereof.

In some embodiments, the present invention provides a composition comprising one or more compounds of Formula I in combination with one or more tyrosine kinase inhibitors. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more tyrosine kinase inhibitors. In certain instances, the effective amount of one or more compounds of Formula I is an amount sufficient to sensitize a tyrosine kinase inhibitor-resistant cancer such as a tyrosine kinase inhibitor-resistant non-small-cell lung cancer (NSCLC) to tyrosine kinase inhibitor therapy. Compounds of Formula I and tyrosine kinase inhibitors can be delivered to a subject via the same route of administration (e.g., orally or parenterally) or via different routes of administration (e.g., intravenously for compounds of Formula I and orally for tyrosine kinase inhibitors, or vice versa).

6. Antigen-Specific Immunotherapeutic Agents

In some embodiments, antigen-specific immunotherapeutic agents include compounds and compositions designed to stimulate the immune system to specifically recognize antigens expressed or overexpressed by cancerous cells. In other embodiments, antigen-specific immunotherapeutic agents include compounds and compositions that will specifically recognize antigens expressed or overexpressed by cancerous cells. Non-limiting examples of antigen-specific immunotherapeutic agents include vaccines (e.g., peptide vaccines), antibodies, cytotoxic T cell lymphocytes (CTLs), chimeric antigen receptor T cells (CAR-T cells), immune checkpoints (e.g., CTLA-4, PD-1, and PD-L1), immune modulating cytokines (e.g., IL-6 and IL-17), and combinations thereof. In particular embodiments, the antigens presented by cancerous cells are highly specific to each cancer type, and the vaccines, antibodies, CTLs, and/or CAR-T cells used is dependent on the cancer type being treated.

A vaccine can stimulate the immune system to specifically recognize and attack antigens presented by cancerous cells. Vaccines can comprise one or more peptides, peptide fragments, fusion peptides, DNA, RNA, other biologic or non-biologic material, or combinations thereof.

In some embodiments, one or more peptides, peptide fragments, or fusion peptides may be used for a peptide vaccine. The peptides may be harvested from an endogenous source or chemically synthesized. The peptides chosen are specific for the type of cancer being treated. For example, when targeting cancer cells, some commonly targeted proteins include GM-CSF, IL-13Rα2, EphA2, and Survivin; however, specific cancer types will have specifically preferred peptides used for targeting afflicted cells. In some embodiments, the one or more peptides in the peptide vaccine are free soluble peptides. In other embodiments, the one or more peptides in the peptide vaccine are tethered together using any means known in the art.

In some embodiments, vaccines include cancer vaccines such as, e.g., tecemotide (L-BLP25), oncophage, sipuleucel-T, and combinations thereof. Tecemotide (L-BLP25) is a liposomal antigen-specific cancer immunotherapy that contains 25 amino acids from the immunogenic tandem-repeat region of MUC1 (see, e.g., Mehta N R et al., Clin. Cancer Res., 18:2861-2871 (2012)).

Antibodies can recognize antigens expressed or overexpressed by cancerous cells. Antigens recognized by these antibodies can be proteins expressed, activated, or overexpressed on the cell surface or proteins secreted into the extracellular fluid. In some embodiments, antibodies can be used to target human effector cells (e.g., macrophages) against the cancerous cells. In some embodiments, antibodies are used to inhibit the normal function of cell surface receptors. In some embodiments, antibodies bind to the ligands of cell surface receptors to block the cellular signaling cascade. Antibodies used as antigen-specific immunotherapeutic agents can be monoclonal or polyclonal antibodies as well as chimeric, humanized, or human antibodies, and can be previously isolated from the patient or produced from another biologic source. Methods of producing antibodies are well known in the art, and may be made by any known means. For example, antibodies described herein can be produced by conventional monoclonal antibody methodology e.g., the standard somatic cell hybridization technique of Kohler and Milstein, Nature 256: 495 (1975), the contents of which are herein incorporated by reference for all purposes. In some embodiments, antibodies useful in the treatment of cancer include immune checkpoint inhibitors. In particular embodiments, antibodies useful in the treatment of cancer include, but are not limited to, alemtuzumab. bevacizumab, cetuximab, ipilimumab, nivolumab, ofatumumab, panitumumab, pembrolizumab, atezolizumab, rituximab, trastuzumab, and combinations thereof.

The use of CTLs and CAR-T cells as antigen-specific immunotherapeutic agents is a form of adoptive T cell transfer therapy. Adoptive T cell transfer therapy is a technique that can boost the natural immune system's ability to combat cancer by enriching for and/or designing T cells that are able to effectively recognize, bind, and kill a diseased cell. CTLs can recognize and bind cancerous cells using T-cell receptors (TCR). TCRs contain a highly variable binding region that allow them to recognize a large range of antigens. TCRs bind to the major histocompatibility complex I (MHC I) of cancerous cells presenting an appropriate antigen. TCRs binding is highly specific, so only a small number of CTLs will be able to recognize a particular antigen. Once an antigen is recognized by CTLs binding to the MHC I complex of the cancerous cell, they activate to induce cellular death. Activated CTLs proliferate to fight the detected cancer.

CTLs administered in this therapy may be derived from the subject or may be derived from other biological sources. Methods for producing CTLs directed to a particular antigen are well known in the art, and can be harvested from an individual possessing a CTL directed to a particular antigen or produced outside of the body (ex vivo). For example, when treating cancer, cytotoxic T cells from a subject's tumor are isolated, the cytotoxic T cells with the greatest antitumor activity are identified, the identified cytotoxic T cells are cultured to produce large amounts of the most effective cells, and the cultured cytotoxic T cells are reintroduced into the subject to treat the cancer. CTLs can also be produced in healthy individuals using ex vivo techniques described in U.S. Pat. No. 5,962,318, and U.S. Patent Application Publication No. 2009/0324539, the contents of which are herein incorporated by reference for all purposes. The ex vivo methods described herein can be useful for individuals both before cancer onset or after cancer onset.

CAR-T cells are modified T cells which have been engineered to possess a cellular specificity domain that has not been produced naturally. The natural specificity domain of T cells are T-cell receptors that recognize a particular antigen presented on MHC class I molecules. In some embodiments, CAR-T cells possess a T-cell receptor that has not been naturally produced in a subject's body. In some embodiments, the cellular specificity domain is a monoclonal antibody that is specific for the targeted cells or tissue. CAR-T cells can be produced using any means known in the art. In some embodiments, cytotoxic T cells are harvested from a subject's blood, the cytotoxic T cells are genetically modified by inserting a gene that encodes for a receptor that recognizes an antigen specific to the cancer affecting the subject, the CAR-T cells are cultured and can be stored for later use or reintroduced into the subject's body to treat the cancer. For more information on the details of producing CAR-T cells, see, e.g., U.S. Pat. Nos. 9,102,760, 8,399,645, 8,975,071, and 8,916,381, the contents of which are herein incorporated by reference for all purposes.

In some embodiments, the present invention provides a composition comprising one or more compounds of Formula I in combination with one or more antigen-specific immunotherapeutic agents. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds of Formula I in combination with one or more antigen-specific immunotherapeutic agents. In certain instances, the effective amount of one or more compounds of Formula I is an amount sufficient to sensitize a cancer that is resistant to treatment with antigen-specific immunotherapeutic agents to such treatment. Compounds of Formula I and antigen-specific immunotherapeutic agents can be delivered to a subject via the same route of administration (e.g., orally or parenterally) or via different routes of administration (e.g., intravenously for compounds of Formula I and orally for antigen-specific immunotherapeutic agents, or vice versa).

C. Diseases and Conditions

In certain aspects, a cancer can be treated or prevented by administering one or more compounds of Formula I. In some embodiments, the one or more compounds of Formula I are administered in combination with an anticancer drug. Cancer generally includes any of various malignant neoplasms characterized by the proliferation of anaplastic cells that tend to invade surrounding tissue and metastasize to new body sites. Non-limiting examples of different types of cancer suitable for treatment using the compositions of the present invention include prostate cancer, lung cancer, ovarian cancer, breast cancer, bladder cancer, thyroid cancer, liver cancer, pleural cancer, pancreatic cancer, cervical cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, skin cancer, choriocarcinomas, head and neck cancers, bone cancer, osteogenic sarcomas, fibrosarcoma, neuroblastoma, glioma, endometrial cancer, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

In particular embodiments, the cancer is an epithelial cancer (e.g., prostate cancer, ovarian cancer, breast cancer, and the like), or a blood cancer (e.g., leukemia, lymphoma, multiple myeloma). In some embodiments, the cancer is a prostate cancer. In certain embodiments, the prostate cancer is an advanced stage prostate cancer selected from one or more of metastatic prostate cancer, drug-resistant prostate cancer (e.g., anti-androgen-resistant prostate cancer such as enzalutamide-resistant prostate cancer, abiraterone-resistant prostate cancer, bicalutamide-resistant prostate cancer, etc.; taxane-resistant prostate cancer; docetaxel-resistant prostate cancer; and the like), hormone refractory prostate cancer, castration-resistant prostate cancer (CRPC), metastatic castration-resistant prostate cancer, AR-V7-induced drug-resistant prostate cancer such as AR-V7-induced anti-androgen-resistant prostate cancer (e.g., AR-V7-induced enzalutamide-resistant prostate cancer), AKR1C3-induced drug-resistant prostate cancer such as AKRC3-induced anti-androgen-resistant prostate cancer (e.g., AKR1C3-induced enzalutamide-resistant prostate cancer), and combinations thereof. In certain embodiments, the prostate cancer is an advanced stage prostate cancer selected from drug-resistant tumors with neuroendocrine (NE) phenotypes or NEPC.

In other embodiments, the cancer is a lung cancer, breast cancer, liver cancer, ovarian cancer, endometrial cancer, bladder cancer, colon cancer, gastric cancer, lymphoma, or a glioma. In certain instances, the lung cancer is a non-small-cell lung cancer (NSCLC), K-Ras mutant lung cancer, BRAF mutant lung cancer, tyrosine kinase inhibitor-resistant lung cancer, small cell lung cancer (SCLC), adenocarcinoma (e.g., adenocarcinoma in situ), squamous cell carcinoma, large cell carcinoma, bronchial carcinoid, or combinations thereof. In certain instances, the breast cancer is triple-negative breast cancer (TNBC), tamoxifen-resistant breast cancer, radiation-resistant breast cancer, ductal carcinoma in situ, invasive ductal carcinoma, HER2-positive breast cancer, ER-positive breast cancer, inflammatory breast cancer, metastatic breast cancer, medullary carcinoma, tubular carcinoma, mucinous carcinoma (colloid), or combinations thereof. In certain instances, the liver cancer is a hepatocellular carcinoma (HCC), cholangiocarcinoma (bile duct cancer), angiosarcoma, hepatoblastoma, or combinations thereof. In certain instances, the glioma is an ependymoma, astrocytoma (e.g., glioblastoma multiforme), oligodendroglioma, brainstem glioma, optic nerve glioma, or combinations thereof (e.g., mixed glioma). In certain instances, the gastric (stomach) cancer is an adenocarcinoma of the distal esophagus, gastroesophageal junction and/or stomach, a gastrointestinal carcinoid tumor, a gastrointestinal stromal tumor, an associated lymphoma, a cancer linked to infection with H. pylori, or combinations thereof.

D. Pharmaceutical Compositions

The pharmaceutical compositions of the present invention encompass compositions made by admixing one or more compounds of Formula I, such as XY018, and a pharmaceutically acceptable carrier and/or excipient or diluent. Such compositions are suitable for pharmaceutical use in an animal or human.

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent(s).

The pharmaceutical compositions of the present invention can include one or more compounds of Formula I (e.g., XY018), one or more anticancer drugs such as an anti-androgen drug (e.g., enzalutamide, abiraterone, and/or bicalutamide) and/or a chemotherapeutic agent (e.g., tamoxifen and/or a taxane such as docetaxel and cabazitaxel), or any pharmaceutically acceptable salts thereof, as an active ingredient, and a pharmaceutically acceptable carrier and/or excipient or diluent. In particular embodiments, the pharmaceutical composition can include one or more compounds of Formula I, such as XY018, and an anti-androgen drug, such as enzalutamide. A pharmaceutical composition may optionally contain other therapeutic ingredients.

The compounds of the present invention can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

In some embodiments, the pharmaceutical compositions comprising one or more compounds of Formula I and the pharmaceutical compositions comprising one or more anticancer drugs are prepared as a single medicament. In other embodiments, the pharmaceutical compositions comprising one or more compounds of Formula I and the pharmaceutical compositions comprising one or more anticancer drugs are prepared as separate medicaments.

The pharmaceutical compositions of the present invention include formulations suitable for topical, parenteral, pulmonary, nasal, rectal, or oral administration. The most suitable route of administration in any given case will depend in part on the nature and severity of the cancer condition and also optionally the stage of the cancer.

In embodiments where the compound of Formula I is administered in combination with an anticancer drug, the administration of the compound of Formula I and the anticancer drug may be administered using the same or a different administration route. For example, in some embodiments, both the compound of Formula I and the anticancer drug may be administered orally or parenterally (e.g., intravenously). For example, in other embodiments, the compound of Formula I may be administered orally, while the anticancer drug may be administered parenterally (e.g., intravenously), or vice versa.

Other preferred compositions include compositions suitable for systemic (enteral or parenteral) administration. Systemic administration includes oral, rectal, sublingual, or sublabial administration. In some embodiments, the compositions may be administered via a syringe or intravenously.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

Compositions for systemic administration include, but are not limited to, dry powder compositions consisting of the composition as set forth herein and the powder of a suitable carrier and/or excipient. The compositions for systemic administration can be represented by, but not limited to, tablets, capsules, pills, syrups, solutions, and suspensions.

In some embodiments, the present invention provides compositions further including a pharmaceutical surfactant. In other embodiments, the present invention provides compositions further including a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

In some embodiments, the present invention provides a pharmaceutical composition including one or more compounds of Formula I, such as XY018, and a pharmaceutically acceptable excipient. In some embodiments, the present invention provides a pharmaceutical composition including one or more compounds of Formula I, such as XY018, and one or more anticancer drugs such as an anti-androgen drug (e.g., enzalutamide, abiraterone, and/or bicalutamide) and/or a chemotherapeutic agent (e.g., tamoxifen and/or a taxane such as docetaxel), in combination with a pharmaceutically acceptable excipient. In particular embodiments, the present invention provides a pharmaceutical composition including one or more compounds of Formula I, such as XY018, and an anti-androgen drug, such as enzalutamide, in combination with a pharmaceutically acceptable excipient. In some of these embodiments, the pharmaceutically acceptable excipient includes a salt or a diluent.

In some embodiments, the present invention provides compositions including an effective amount of one or more compounds of Formula I, such as XY018. In some embodiments, the composition is formulated for oral administration or parenteral (e.g., intravenous) administration and includes one or more compounds of Formula I, such as XY018, and at least one member selected from the group consisting of an aqueous solution and a buffer solution. In some embodiments, the composition can include an effective amount of one or more compounds of Formula I, such as XY018, and one or more anticancer drugs such as an anti-androgen drug (e.g., enzalutamide, abiraterone, and/or bicalutamide) and/or a chemotherapeutic agent (e.g., tamoxifen and/or a taxane such as docetaxel).

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethyleneglycol 6000 and titatium dioxide. Tablets may be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Controlled release parenteral formulations of the compositions of the present invention can be made as implants, oily injections, or as particulate systems. For a broad overview of delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles.

Polymers can be used for ion-controlled release of compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

E. Methods of Administration

Pharmaceutical compositions or medicaments comprising one or more compounds of Formula I can be administered to a subject at a therapeutically effective dose to treat the subject's cancer, as described herein. In some embodiments, pharmaceutical compositions or medicaments comprising one or more compounds of Formula I can be co-administered to a subject in combination with an effective amount of an anticancer drug at a therapeutically effective dose to treat the subject's cancer, as described herein. In some embodiments, the pharmaceutical composition or medicament comprising one or more compounds of Formula I is administered to a subject in an amount sufficient in to elicit an effective therapeutic response in the subject. In some embodiments, the pharmaceutical composition or medicament comprising one or more compounds of Formula I can be co-administered to a subject at a therapeutically effective dose in combination with an effective amount of an anticancer drug to elicit an effective therapeutic response in the subject.

In certain methods of treating cancer, set forth herein, the methods comprise first administering one or more compounds of Formula I, such as XY018, to a patient having cancer, and then administering an anticancer drug, such as an anti-androgen drug and/or a chemotherapeutic agent, to the patient. In certain methods of treating cancer, set forth herein, the methods comprise first administering an anticancer drug, such as an anti-androgen drug and/or a chemotherapeutic agent, to a patient having cancer, and then administering one or more compounds of Formula I, such as XY018, to the patient. In certain methods of treating cancer, set forth herein, the methods comprise co-administering one or more compounds of Formula I, such as XY018, with an anticancer drug, such as an anti-androgen drug and/or a chemotherapeutic agent, to a patient having cancer.

In some embodiments, the methods of administration comprise administering one or more compounds of Formula I, such as XY018, alone or in combination with enzalutamide to a patient in need thereof. In other embodiments, the methods of administration comprise administering one or more compounds of Formula I, such as XY018, alone or in combination with abiraterone to a patient in need thereof. In yet other embodiments, the methods comprise administering one or more compounds of Formula I, such as XY018, alone or in combination with bicalutamide to a patient in need thereof. In still yet other embodiments, the methods comprise administering one or more compounds of Formula I, such as XY018, alone or in combination with a taxane such as docetaxel to a patient in need thereof. In further embodiments, the methods comprise administering one or more compounds of Formula I, such as XY018, alone or in combination with tamoxifen to a patient in need thereof.

In certain embodiments, the present invention provides a method of delivering an effective amount of one or more compounds of Formula I, such as XY018, to a patient having cancer such as prostate cancer (e.g., CRPC).

The compounds of Formula I described herein are useful in the manufacture of a pharmaceutical composition or a medicament. A pharmaceutical composition or medicament can be administered to a subject in need thereof, e.g. a patient having a cancer such as prostate cancer (e.g., CRPC), lung cancer, breast cancer, liver cancer, ovarian cancer, endometrial cancer, bladder cancer, colon cancer, gastric cancer, lymphoma, or a glioma.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E.W. Martin. Compounds and agents of the present invention and their physiologically acceptable salts and solvates can be formulated for administration by any suitable route, including via inhalation, topically, nasally, orally, intravenously, parenterally, or rectally.

1. Routes of Administration

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. The present invention provides tablets and gelatin capsules comprising one or more compounds of Formula I, such as XY018, alone or in combination with other compounds such as anti-androgen drugs and/or docetaxel, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient(s).

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

In particular embodiments, a pharmaceutical composition or medicament of the present invention can comprise (i) an effective amount of one or more compounds of Formula I, such as XY018, and (ii) optionally an anticancer drug such as an anti-androgen drug (e.g., enzalutamide, abiraterone, bicalutamide), a chemotherapeutic agent such as a taxane (e.g., docetaxel) or tamoxifen, and combinations thereof. The therapeutic agent(s) may be used individually, sequentially, or in combination with one or more other such therapeutic agents (e.g., a first therapeutic agent, a second therapeutic agent, a compound of the present invention, etc.). Administration may be by the same or different route of administration or together in the same pharmaceutical formulation.

2. Dosage

Pharmaceutical compositions or medicaments can be administered to a subject at a therapeutically effective dose to prevent, treat, sensitize, or control a cancer such as prostate cancer (e.g., CRPC), lung cancer, breast cancer (e.g., TNBC), liver cancer, ovarian cancer, endometrial cancer, bladder cancer, colon cancer, gastric cancer, lymphoma, or a glioma as described herein. The pharmaceutical composition or medicament is administered to a subject in an amount sufficient to elicit an effective therapeutic response in the subject. An effective therapeutic response includes a response that at least partially arrests or slows the symptoms or complications of the cancer. An amount adequate to accomplish this is defined as a “therapeutically effective dose.”

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. A unit dosage for oral administration to a mammal of about 50 to about 70 kg may contain between about 5 and about 500 mg, about 25-200 mg, about 100 and about 1000 mg, about 200 and about 2000 mg, about 500 and about 5000 mg, or between about 1000 and about 2000 mg of the active ingredient. A unit dosage for oral administration to a mammal of about 50 to about 70 kg may contain about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of a compound of Formula I, such as XY018, to a human being following established protocols known in the art and the disclosure herein.

The data obtained from, for example, animal studies (e.g., rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the ICs. (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a composition is from about 1 ng/kg to about 100 mg/kg for a typical subject.

A typical composition of the present invention for oral or intravenous administration can be about 0.1 to about 10 mg of active ingredient per patient per day; about 1 to about 100 mg per patient per day; about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient per patient per day. Methods for preparing administrable compositions will be known or apparent to those skilled in the art and are described in more detail in such publications as Remington: The Science and Practice of Pharmacy, 21^{st} Ed., University of the Sciences in Philadelphia, Lippencott Williams & Wilkins (2005).

Exemplary doses of the compositions described herein include milligram or microgram amounts of the composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, a pharmaceutical composition or medicament of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. A preferred dose is about 2, 3, 4, 5, 6, 7, 8, 9, 10, 12, 15, 18, 20, 25, 30, 40, or 50 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, compounds or agents described herein may be administered for multiple days at the therapeutically effective daily dose. Thus, therapeutically effective administration of compounds to treat prostate cancer in a subject may require periodic (e.g., daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

In some embodiments, the compound of Formula I is orally administered. In some embodiments, the compound of Formula I (e.g., XY018) is orally administered to a subject (e.g., an adult human) at a daily dose of approximately 100; 200; 300; 400; 500; 600; 700; 800; 900; 1,000; 1,250; 1,500; 1,750; 2,000; 2,500; 3,000; 3,500; 4,000; 4,500; 5,000; or more mg per day. In some embodiments, the compound of Formula I (e.g., XY018) is orally administered to a subject (e.g., an adult human) at a daily dose of between 1,000 and 2,000 mg per day. In some embodiments, the compound of Formula I (e.g., XY018) is orally administered. In some embodiments, the compound of Formula I (e.g., XY018) is orally administered to a subject (e.g., an adult human) at a daily dose of approximately 10, 15, 20, 25, 30, 35, 40, 50, 60, 70, 80, 90, 100, 125, 150, 175, 200, 225, 250, 300, 350, 400, 500, or more mg per day. In some embodiments, the compound of Formula I (e.g., XY018) is orally administered to a subject (e.g., an adult human) at a daily dose of between 25 and 200 mg per day. In some embodiments, the compound of Formula I (e.g., XY018) and an anti-androgen drug are orally co-administered. For example, the compound of Formula I (e.g., XY018) can be co-administered at a daily oral dose of between 25 and 1000 mg per day with the anti-androgen drug at a daily oral dose of between 25 and 2000 mg per day.

In some embodiments, the one or more compounds of Formula I is orally administered. In some embodiments, the one or more compounds of Formula I, such as XY018, is orally administered to a subject (e.g., an adult human) at a daily dose of approximately 100; 200; 300; 400; 500; 600; 700; 800; 900; 1,000; 1,250; 1,500; 1,750; 2,000; 2,500; 3,000; 3,500; 4,000; 4,500; 5,000; or more mg per day. In some embodiments, the one or more compounds of Formula I, such as XY018, is orally administered to a subject (e.g., an adult human) at a daily dose of between 1,000 and 2,000 mg per day. In some embodiments, the one or more compounds of Formula I, such as XY018, is orally administered to a subject (e.g., an adult human) at a daily dose of approximately 10, 15, 20, 25, 30, 35, 40, 50, 60, 70, 80, 90, 100, 125, 150, 175, 200, 225, 250, 300, 350, 400, 500, or more mg per day. In some embodiments, the one or more compounds of Formula I, such as XY018, is orally administered to a subject (e.g., an adult human) at a daily dose of between 25 and 200 mg per day. In some embodiments, the one or more compounds of Formula I, such as XY018, and an anticancer drug are orally co-administered. For example, the one or more compounds of Formula I, such as XY018, can be co-administered at a daily oral dose of between 25 and 1000 mg per day with the anticancer drug at a daily oral dose of between 25 and 2000 mg per day.

In some embodiments, the methods comprise sequentially administering one or more compounds of Formula I, such as XY018, followed by one or more anticancer drugs such as an anti-androgen drug (e.g., enzalutamide, abiraterone, bicalutamide), a chemotherapeutic agent such as a taxane (e.g., docetaxel) or tamoxifen, and combinations thereof. In some embodiments, the methods comprise sequentially administering one or more anticancer drugs followed by one or more compounds of Formula I, such as XY018.

Following successful treatment, it may be desirable to have the subject undergo maintenance therapy to prevent the recurrence of the cancer.

Determination of an effective amount is well within the capability of those skilled in the art, especially in light of the detailed disclosure provided herein. Generally, an efficacious or effective amount of an composition is determined by first administering a low dose or small amount of the composition, and then incrementally increasing the administered dose or dosages, adding a second or third medication as needed, until a desired effect of is observed in the treated subject with minimal or no toxic side effects.

Single or multiple administrations of the compositions are administered depending on the dosage and frequency as required and tolerated by the patient. In any event, the composition should provide a sufficient quantity of the compositions of this invention to effectively treat the patient. Generally, the dose is sufficient to treat or ameliorate symptoms or signs of disease without producing unacceptable toxicity to the patient.

F. Kits, Containers, Devices, and Systems

A wide variety of kits and systems can be prepared according to the present invention, depending upon the intended user of the kit and system and the particular needs of the user. In some embodiments, the present invention provides a kit that includes one or more compounds of Formula I. In other aspects, the present invention provides a kit that includes one or more compounds of Formula I and one or more anticancer drugs such as an anti-androgen drug (e.g., enzalutamide, abiraterone, and/or bicalutamide) and/or a chemotherapeutic agent (e.g., tamoxifen and/or a taxane such as docetaxel).

Some of the kits described herein can include a label describing a method of administering one or more compounds of Formula I and/or one or more anticancer drugs. Some of the kits described herein can include a label describing a method of treating cancer in a subject with a cancer such as prostate cancer (e.g., CRPC), lung cancer, breast cancer (e.g., TNBC), liver cancer, ovarian cancer, endometrial cancer, bladder cancer, colon cancer, gastric cancer, lymphoma, or a glioma.

The compositions of the present invention, including but not limited to, compositions comprising one or more compounds of Formula I and optionally one or more anticancer drugs may, if desired, be presented in a bottle, jar, vial, ampoule, tube, or other container-closure system approved by the Food and Drug Administration (FDA) or other regulatory body, which may provide one or more dosages containing the compounds. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

In some embodiments, the kit includes a container which is compartmentalized for holding the various elements of a formulation (e.g., the dry ingredients and the liquid ingredients) or composition, instructions for making the formulation or composition, and instructions for administering the formulation or composition for enhancing the immune response in a subject with a cancer.

In certain embodiments, the kit may include the pharmaceutical preparation(s) in dehydrated or dry form, with instructions for its rehydration (or reconstitution) and administration.

Kits with unit doses of the compounds described herein, e.g. in oral, rectal, transdermal, or injectable doses (e.g., for intramuscular, intravenous, or subcutaneous injection), are provided. In such kits, an informational package insert describing the use and attendant benefits of the composition for enhancing the immune response in a subject with a cancer such as prostate cancer (e.g., CRPC), lung cancer, breast cancer, liver cancer, ovarian cancer, endometrial cancer, bladder cancer, colon cancer, gastric cancer, lymphoma, or a glioma may be included in addition to the containers containing the unit doses.

Some embodiments of the present invention provide packages that include one or more compounds of Formula I and optionally one or more anticancer drugs such as an anti-androgen drug (e.g., enzalutamide, abiraterone, and/or bicalutamide) and/or a chemotherapeutic agent (e.g., tamoxifen and/or a taxane such as docetaxel).

### IV. Examples

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1. Synthesis of XY018

This example illustrates the synthesis of XY018 (N-(2′-fluoro-4′-(1,1,1,3,3,3-hexafluoro-2-hydroxypropan-2-yl)-[1,1′-biphenyl]-4-yl)-2-(2-nitrophenyl)acetamide)) from commercially available compounds, shown in Scheme 1.

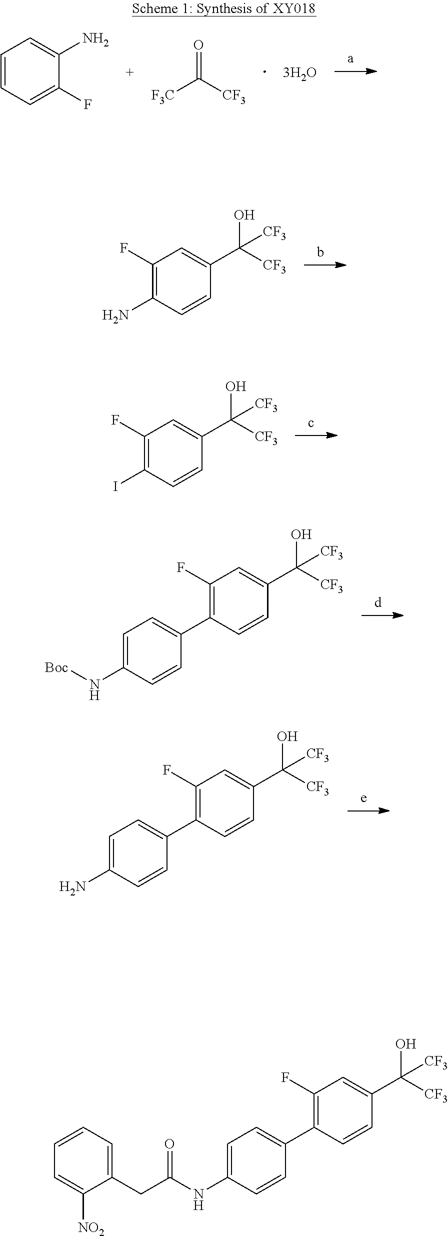

The reaction conditions and solvents used in each chemical conversion are described below.

Synthesis of the desired reagents and solvents were obtained from commercial suppliers and used without further purification. Flash chromatography was performed using silica gel (300-400 mesh). All reactions were monitored by TLC, using silica gel plates with fluorescence F254 and UV light visualization. ¹H-NMR spectra were recorded on a Bruker AV-400 spectrometer at 400 MHz. Coupling constants (J) are expressed in hertz (Hz). Chemical shifts (δ) of NMR are reported in parts per million (ppm) units relative to internal control (TMS). The low- or high-resolution of ESI-MS was recorded on an Agilent 1200 HPLC-MSD mass spectrometer or Applied Biosystems Q-STAR Elite ESI-LC-MS/MS mass spectrometer, respectively.

Step (a): To 2-fluoroaniline (6 g, 54 mmol) in a pressure vessel was added hexafluoroacetone trihydrate (12.5 g, 56.7 mmol) and p-toluenelsuphonic acid (0.85 g, 5.4 mmol). The reaction mixture was stirred at 90° C. for 12 h. Added water, extracted with ethyl acetate, the organic layer was washed with saturated NaHCO₃ solution and brine and dried over Na₂SO₄. The solid was filtered off, and the filtrate was concentrated under reduced pressure. The resulting crude product was purified by silica gel chromatography with petroleum ether/ethyl acetate (10/1, v/v) to yield 2-(4-Amino-3-fluorophenyl)-1,1,1,3,3,3-hexafluoropropan-2-ol as white solid (4.45 g, 30% yield). MS (ESI), m/z for C₉H₆F₇NO ([M+1]⁺): Calcd 277.14, found 278.0.

Step (b): To a solution of 2-(4-amino-3-fluorophenyl)-1,1,1,3,3,3-hexafluoropropan-2-ol (4.45 g, 16.1 mmol) in DMF (100 mL) was added concentrated HCl (18 mL, 73 mmol) and sodium nitrite (1.66 g, 24 mmol), dissolved in H₂O (20 mL) at 0° C. The reaction mixture was stirred at 0° C. for 30 min, then added potassium iodide (4 g, 24 mmol) in portions, and then the reaction mixture was stirred at room temperature for overnight. Added ethyl acetate, washed with a saturated NaHCO₃ solution and brine and dried over Na₂SO₄. The solid was filtered off, and the filtrate was concentrated under reduced pressure. The resulting crude product was purified by silica gel chromatography with petroleum ether/ethyl acetate (50/1, v/v) to yield 1,1,1,3,3,3-hexafluoro-2-(3-fluoro-4-iodophenyl)propan-2-ol (6.2 g, 96% yield). H NMR: (400 MHz, d-DMSO) δ 7.31 (d, J=8.4 Hz, 1H), 7.48 (d, J=1.6 Hz, 9.2 Hz, 1H), 8.05 (dd, J=6.8 Hz, 8.4 Hz, 1H), 9.07 (s, 1H).

Step (c): To a solution of 1,1,1,3,3,3-hexafluoro-2-(3-fluoro-4-iodophenyl)propan-2-ol (6.2 g, 16 mmol) in 1,4-dioxane (100 mL) and water (20 mL) was added (4-((tert-butoxycarbonyl)amino)phenyl)boronic acid (4.2 g, 17.6 mmol), followed by addition of potassium carbonate (6.6 g, 48 mmol) and Pd(PPh₃)₄ (0.9 g, 0.78 mmol), the vessel was purged with argon, sealed and heated to 80° C. for 5 h. Added water, extracted with ethyl acetate, the organic layer was washed with brine and dried over Na₂SO₄. The solid was filtered off, and the filtrate was concentrated under reduced pressure. The resulting crude product was purified by silica gel chromatography with petroleum ether/ethyl acetate (20/1, v/v) to yield tert-butyl(2′-fluoro-4′-(1,1,1,3,3,3-hexafluoro-2-hydroxypropan-2-yl)-[1,1′-biphenyl]-4-yl)carbamate (5.35 g, 74% yield). ¹H NMR (400 MHz, CDCl₃) δ 7.54-7.44 (m, 6H), 7.40 (d, J=8.4 Hz, 1H), 6.57 (s, 1H), 3.82 (s, 1H), 1.53 (d, J=3.2 Hz, 9H).

Step (d): To a solution of tert-butyl(2′-fluoro-4′-(1,1,1,3,3,3-hexafluoro-2-hydroxypropan-2-yl)-[1,1′-biphenyl]-4-yl)carbamate (5.35 g, 11.8 mmol) in DCM (50 mL) was added trifluoroacetic acid (7 mL, 96 mmol) dropwise at 0° C. The reaction mixture was stirred at room temperature for 3 h. The mixture was concentrated under reduced pressure and recrystallisation was then carried out in petroleum ether and ethyl acetate and 2-(4′-amino-2-fluoro-[1,1′-biphenyl]-4-yl)-1,1,1,3,3,3-hexafluoropropan-2-ol was isolated (3.8 g, 91% yield). ¹H NMR (400 MHz, d-DMSO) δ 8.91 (s, 1H), 7.61 (t, J=8.4 Hz, 1H), 7.50-7.47 (m, 2H), 7.30 (d, J=7.2 Hz, 2H), 6.65 (d, J=8.4 Hz, 2H), 5.41 (s, 2H).

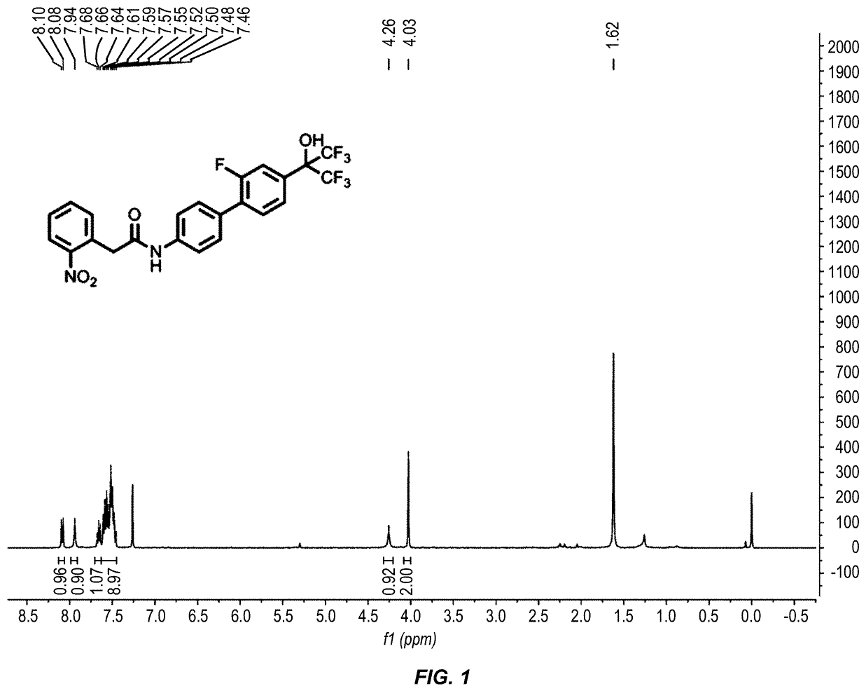

Step (e): To a solution of 2-(2-nitrophenyl) acetic acid (56 mg, 0.31 mmol) in DCM (20 mL) was added HATU (213 mg, 0.56 mmol) and DIPEA (0.5 mL). The mixture was stirred at room temperature for 5 min, then added 2-(4′-amino-2-fluoro-[1,1′-biphenyl]-4-yl)-1,1,1,3,3,3-hexafluoropropan-2-ol (100 mg, 0.28 mmol). The reaction mixture was stirred at room temperature for 3 h. Added water, extracted with ethyl acetate, the organic layer was washed with brine and dried over Na₂SO₄. The solid was filtered off, and the filtrate was concentrated under reduced pressure. The resulting crude product was purified by silica gel chromatography with petroleum ether/ethyl acetate (4/1, v/v) to yield N-(2′-fluoro-4′-(1,1,1,3,3,3-hexafluoro-2-hydroxypropan-2-yl)-[1,1′-biphenyl]-4-yl)-2-(2-nitrophenyl)acetamide (118 mg, 81% yield). H NMR (400 MHz, CDCl₃) δ 8.09 (d, J=8.4 Hz, 1H), 7.94 (s, 1H), 7.66 (t, J=7.6 Hz, 1H), 7.61-7.46 (m, 9H), 4.26 (s, 1H), 4.03 (s, 2H). MS (ESI), m/z for C₂₃H₁₅F₇N₂O₄ ([M+1]⁺): Calcd 516.37, found 517.0 (FIG. 1).

### Example 2. RORγ Antagonist XY018 Inhibits Growth and Survival of CRPC Cells

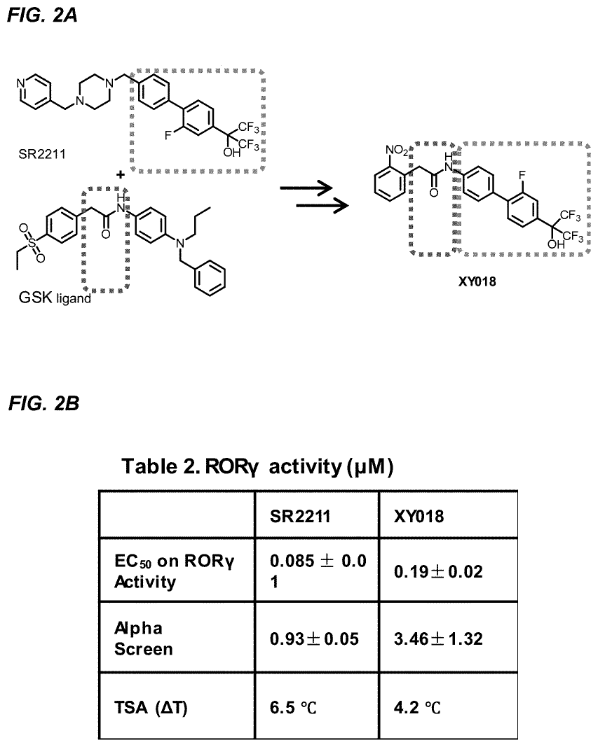

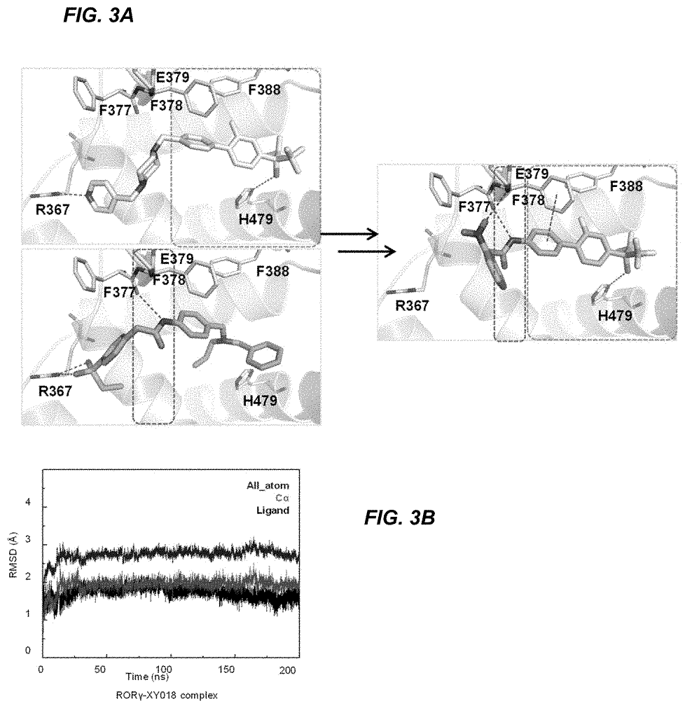

By combining some of the structural features of SR2211 and GSK805, we developed compound XY018 that displayed a high potency (EC₅₀, 190 nM) in inhibition of RORγ constitutive activity (FIG. 2). Molecular docking demonstrated that XY018 may bind to RORγ hydrophobic ligand binding domain (LBD) through several conserved hydrogen bonds and hydrophobic interactions. For example, the phenyl group in the middle likely forms a π-π interaction with the side chain of Phe378 of the LBD, while the amide group can form a direct hydrogen bond with Phe377. The nitro group and hydroxyl group at the two ends may form hydrogen bonds with Glu379 and His479, respectively. Molecular dynamics simulation demonstrated that the RORγ-XY018 complex is very stable with these interactions (FIG. 3).

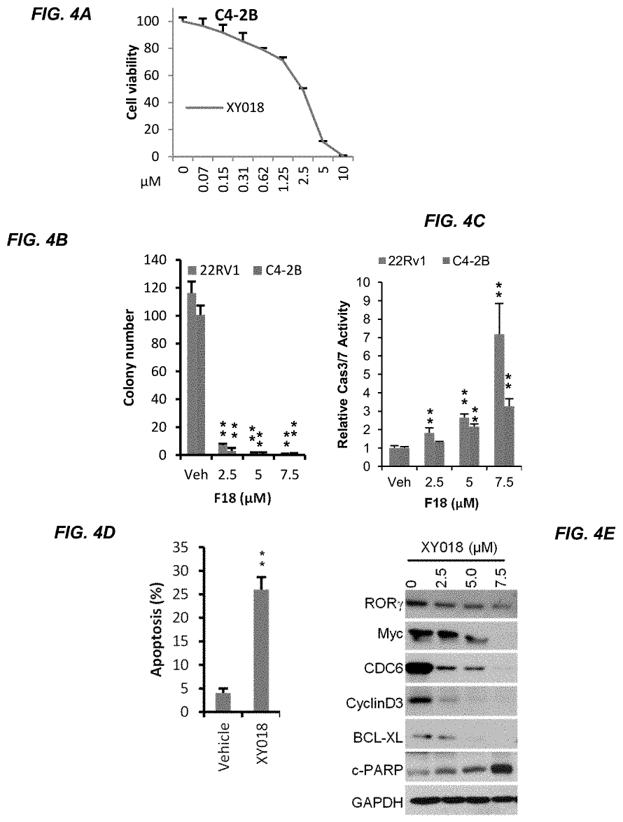

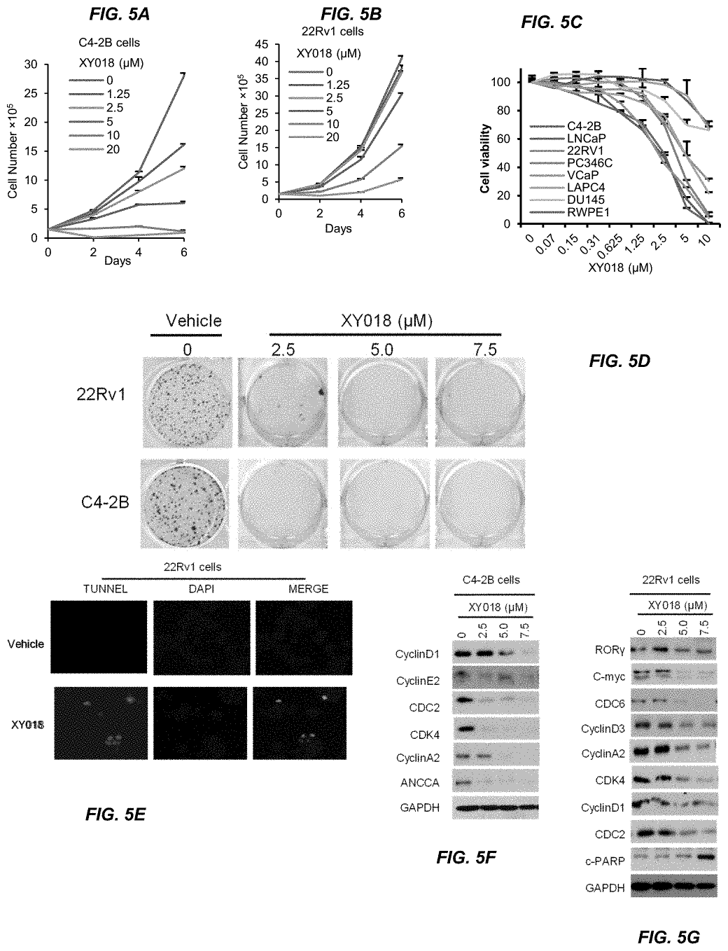

XY018 also displayed strong growth inhibition with much higher potencies than that of enzalutamide. XY018 showed inhibitory potency at low μMs in LNCaP cells (2.1 μM respectively (FIG. 4A; FIGS. 5A, B, and C). Moreover, strong growth inhibition by XY018 was observed in other AR-positive PCa cell models including 22Rv1, VCaP, LNCaP, LAPC4 and PC346C (FIGS. 5A, B, and C). Consistent with the cell death effect by RORγ gene knockdown, treatment of C4-2B and 22Rv1 cells with antagonists XY018 elicited a pronounced inhibition of cell survival as shown by poor colony formation and marked apoptosis (FIG. 4B-D; FIGS. 5D and 5E). In line with the cellular effects, the antagonist strongly inhibited the expression of key proliferation and survival proteins, including Myc (FIG. 4E; FIGS. 5F and 5G).

### Example 3. Inhibition of RORγ with XY018 Strongly Suppresses AR and its Variant Expression

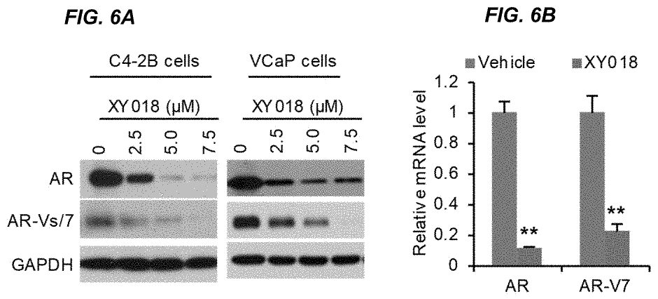

Immunoblotting and qRT-PCR analysis were used to determine the effect of XY018 mediated RORγ inhibition on the expression of AR and AR-V7 in C4-2B or AR-V7 in VCaP cells. FIG. 6 illustrates that both AR and AR-V7 expression is inhibited by XY018 in dose-dependent manner.

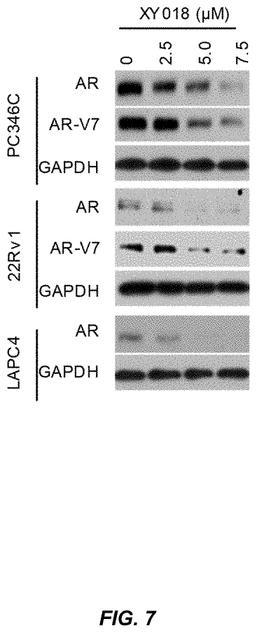

Immunoblotting of other AR-positive cancer cells 22Rv1, LAPC4, and PC346C also demonstrated potent does dependent XY018 inhibition of AR expression (FIG. 7).

### Example 4. XY018 Inhibits Tumor Growth

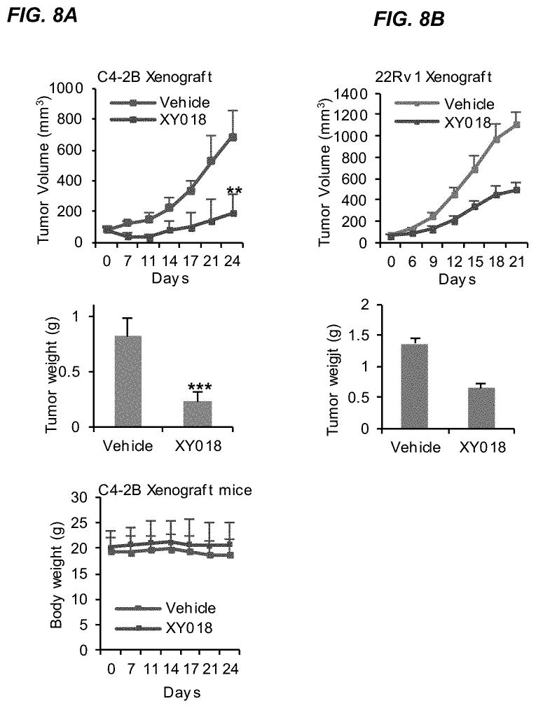

C4-2B and 22Rv1 xenografts were treated with XY018 or a control treatment lacking XY018 for 21-24 days. Over the course of treatment strong tumor growth inhibition was observed with XY018 in the C4-2B and 22Rv1 xenograft tumor models (FIG. 8).

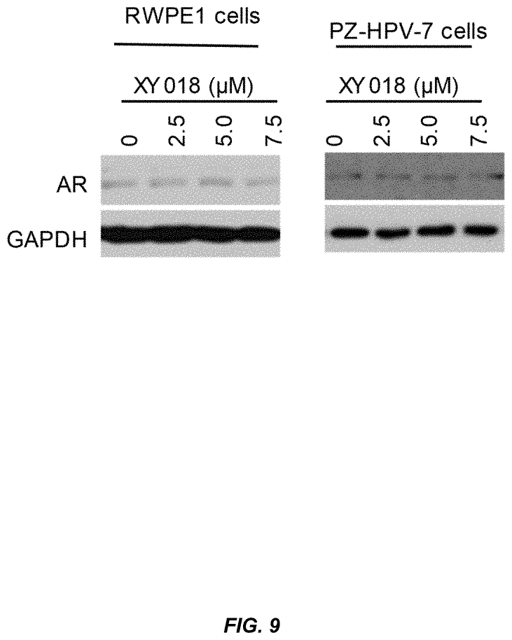

XY018 did not have any significant effect on the endogenous AR expression in two non-malignant human prostate epithelial cells. FIG. 9 illustrates an immunoblotting analysis of RORγ and AR expression in non-malignant, human prostate epithelial RWPE1 and PZ-HPV7 cells with indicated treatments showing that XY018 does not affect AR expression.

### Materials and Methods

### Cell Culture

LNCaP, C4-2B, 22Rv, PC-3, and PC346C prostate cancer cells were cultured in RPMI1640, VCaP, HEK293T and human fibroblast IMR₉₀ cells were in DMEM, LAPC-4 was in Iscove's MEM (all from Corning), and RWPE-1 and PZ-HPV-7 were in Keratinocyte Serum Free Medium (K-SFM) (Invitrogen) with the supplements. All the culture media except for RWPE-1 and PZ-HPV-7 were supplemented with 10% FBS (Hyclone) except indicated otherwise. For experiments, C4-2B cells were cultured in RPMI supplemented with 9% cds-FBS plus 1% regular FBS (to mimic the CRPC condition) unless indicated otherwise and 22Rv1 cells were cultured in RPMI supplemented with 10% cds-FBS. Cells were grown at 37° C. in 5% CO2 incubators. LNCaP, VCaP, 22Rv1, PC-3, 293T, IMR90, PZ-HPV-7 and RWPE-1 were from ATCC. C4-2B was from UroCor Inc. (Oklahoma City, Okla.). LAPC4 and PC346C were kindly provided respectively by Dr. Charles Sawyers (MSKCC, New York) or by Dr. Adrie van Bokhoven (University of Colorado). The prostate cancer cell lines were recently authenticated by ATCC using STR profiling. Cell lines were regularly tested being negative for mycoplasma.

qRT-PCR and Immunoblotting Analysis

Total RNA was isolated from cells in 6-well or 10-cm plates or from xenograft tumors, and the cDNA was prepared, amplified and measured in the presence of SYBR as described in Yang, P., et al., Molecular and cellular biology 32, 3121-3131 (2012). Briefly, the fluorescent values were collected and a melting curve analysis was performed. Fold difference was calculated as described previously Yang, P., et al. (see, id). The experiments were performed at least three times with data presented as mean values±s.d. Cell lysates were analyzed by immunoblotting with antibodies specifically recognizing RORγ, AR, AR-V7 and the indicated proteins.

### Molecular Docking & Molecular Dynamics Simulation

Schrödinger 2014 Suite was used to predict the potential binding mode of RORγ and its ligands. The crystal structure of RORγ LBD in complex with antagonist (PDB code: 4QM0.pdb) was used as the reference structure in the docking study. Protein structure preparation for docking studies included water deletion, hydrogen atom addition and protonation state adjustment. All of the ligand and protein preparation were performed in Maestro (version 9.9, Schrödinger, LLC, New York, N.Y., 2014) implemented in the Schrödinger program (http://www.schrödinger.com). In this study, ligands were prepared using the Ligprep module to obtain energy minimized 3D structures, which were then docked into ligand binding pocket with the Glide molecular docking program (version 6.4, Schrödinger, LLC, New York, N.Y., 2014) using the Glide SP, and Glide XP modes. For all of the methods, Glide docks flexible ligands into a rigid receptor structure. Final ranking from the docking was based on the docking score, which combines the Epik state penalty with the Glide Score. Finally, the binding poses with the top glide score (20 poses) were clustered and selected for further visual evaluation and molecular dynamics simulations.

Molecular dynamics (MD) simulations were conducted by using AMBER 14 program. The starting coordinates were obtained from docking results. For ligand coordinates, top scored representative poses of each cluster were chosen. For protein coordinates, the protein preparation panel in Schrodinger 2014 Suite was applied to assign the protonation states and orientations of residues, which was then further processed by using LEaP module in Amber program. Parameters of compounds were prepared by AM-bcc model and the other parameters were assigned from the AMBER GAFF force field using ANTECHAMBER. Topology and parameter files for the protein, ligand and complex were generated using the LEaP module in AMBER 14. TIP3PBOX water molecules were added in cube periodic boxes, which were 10 Å×10 Å×10 Å. To ensure overall neutrality of the system, appropriate Na⁺ and Cl⁻ were added at physiological concentration in the box. For each system, energy minimization and MD simulation were carried out using the GPU version of the PMEMD program in AMBER 14 program. The MD simulations were performed for up to 200 ns for each complex system. The coordinates of the complexes were saved every 2 ps, those snapshots were taken in production run for detailed analysis. Trajectories were analyzed using the PTRAJ module in Amber 14.

### AlphaScreen Assay

The human RORγ (ligand binding domain) LBD (residues 262-507) was expressed as a His6-fusion protein (“His6” disclosed as SEQ ID NO: 1) using the pET24a expression vector (Novagen, Madison, Wis.) as described in Kojetin, D. J. et al., Nature reviews. Drug discovery 13, 197-216 (2014). Interactions between RORγ and ligands were assessed by luminescence-based AlphaScreen technology (Perkin Elmer) using a histidine detection kit from PerkinElmer (Norwalk, Conn.). All of the reactions contained 100 nM receptor LBD bound to nickel acceptor beads (5 μg/mL) and 20 nM biotinylated SRC1-4 peptide bound to streptavidin donor beads (5 μg/mL) in the presence or absence of the indicated amounts of control compounds SR2211 or candidate compounds. The N-terminal biotinylated coactivator peptide SRC1-4 sequence was QKPTSGPQTPQAQQKSLLQQLLTE (SEQ ID NO: 2). Compound concentrations varied from 150 nM to 200 μM in the dose-response assay.

### Thermal Shift Assay

All reactions were carried out using a CFX96 real-time PCR system (Bio-Rad). Protein were buffered in 10 mM of HEPES, pH 7.5, 150 mM of NaCl and 5% glycerol at a concentration of 7.5 μM. Compounds were added at a final concentration of 200 μM. All assays were set up in 20 μL final reaction volume in 96-well plate with 10×SYPRO Orange (Invitrogen) and incubated with compounds on ice for 30 mins. The samples were heated from 30° C. to 90° C. with a thermal ramping rate of 1° C./min and the fluorescence signals were read out. ΔTm was recorded as the difference between the transition midpoints of sample and reference wells containing protein without ligand in the same plate.

### Example 5. Targeting of the Nuclear Receptor RORγ with the Receptor-Specific, Small Molecule Inhibitors XY018 in Tumor Cells of Different Human Cancers

The following example illustrates how the RORγ inhibitor XY018 can be used in the treatment of numerous different cancer types including prostate cancer, lung cancer, breast cancer, liver cancer, ovarian cancer, endometrial cancer, bladder cancer, colon cancer, lymphoma, and glioma.

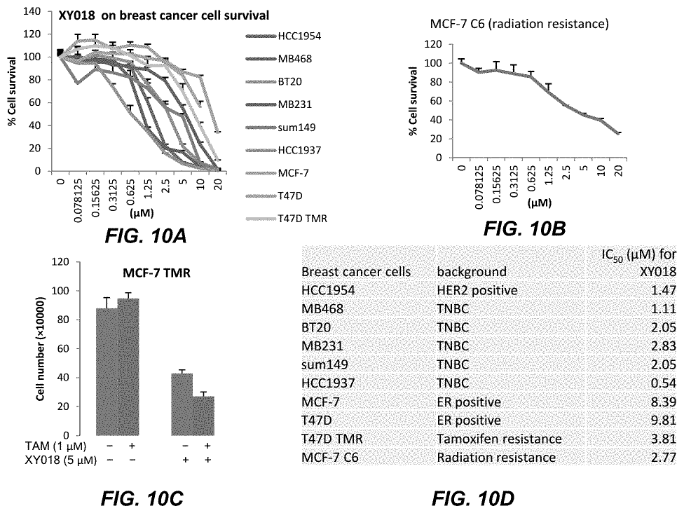

Using a CellTiter-GLO assay, the viability of cells of different cancer types was measured after treatment with different concentrations of the RORγ inhibitor XY018 for 4 days. FIG. 10A shows that XY018 strongly inhibited the growth and survival of breast cancer cells with different major molecular features, with triple-negative breast cancer (TNBC) cells being more sensitive than the ER-positive breast cancer cells. In particular, XY018 displayed an IC50 ranging from 0.5 μM to less than 3 μM in TNBC cells including MDA-MB468, MDA-MB231, BT20, SUM149 and HCC1937 (FIG. 10D). XY018 also displayed relatively strong inhibition of HER2-positive cells, such as HCC1954, with an IC50 of 1.47 μM (FIG. 10D). In addition, XY018 displayed significant inhibition of ER-positive cells, such as MCF-7 and T47D, with an IC50 around 9 μM (FIG. 10D).

FIGS. 10B and 10C show that the RORγ inhibitor XY018 was effective in potently inhibiting the growth and survival of human breast cancer cells that are resistant to different therapies including radiation (MCF-7-C6) and targeted therapies such as tamoxifen (MCF-7-TamR). In the case of the tamoxifen-resistant MCF-7-TamR cell line, tamoxifen (1 μM of 4(OH)-tamoxifen) alone did not show any inhibitory effect; in fact, it showed a slight stimulation. However, tamoxifen displayed a synergistic growth inhibitory effect on tamoxifen-resistant breast cancer cells when used in combination with XY018. As such, XY018 was able to sensitize tamoxifen-resistant breast cancer cells to tamoxifen.

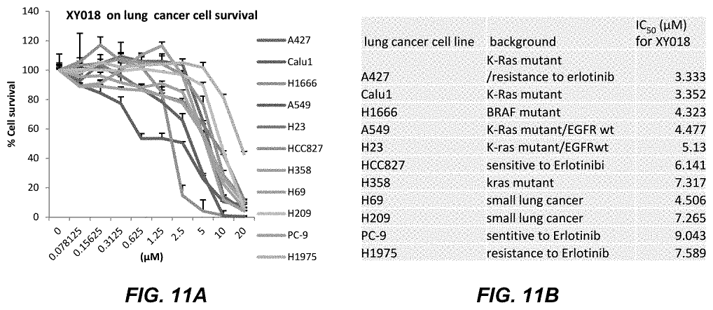

FIG. 11A shows that the RORγ inhibitor XY018 strongly inhibited the growth and survival of lung cancer cells with different molecular and histological features, including cells with an oncogenic mutant KRAS gene (e.g., A427, Calu1, A549, H23, and H358), cells with an oncogenic BRAF mutant gene (e.g., H1666) and cells with an EGFR mutant gene (e.g., HCC827 and PC-9). All those cells are derived from human tumors of non-small cell lung carcinoma (NSCLC). XY018 was also effective in the inhibition of cells derived from human tumors of small cell lung carcinoma (SCLC) such as H69 and H209. The IC50s of XY018 in the above lung cancer cells range from about 3.3 μM to about 9.0 μM (FIG. 11B).

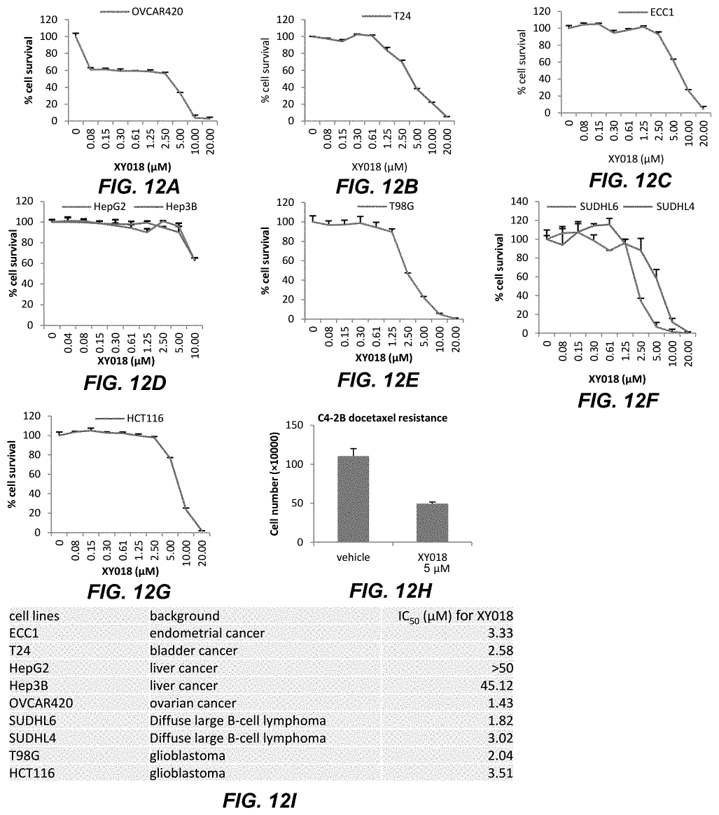

FIGS. 12A-12I show that the RORγ inhibitor XY018 displayed significant inhibition of the growth and survival of ovarian cancer cells (e.g., OVCAR420), bladder cancer cells (e.g., T24), endometrial cancer cells (e.g., ECC1), glioblastoma cells (e.g., T98G), diffuse large B cell lymphoma (DLBCL) (e.g., SUDHL4 and SUDHL6), colon cancer cells (e.g., HCT116), and docetaxel-resistant cells (e.g., C4-2B), with IC50s less than about 3.5 μM.

### Example 6. Series of RORγ Inhibitor Compounds of Formula I Inhibit Human Prostate Cancer Cell Growth

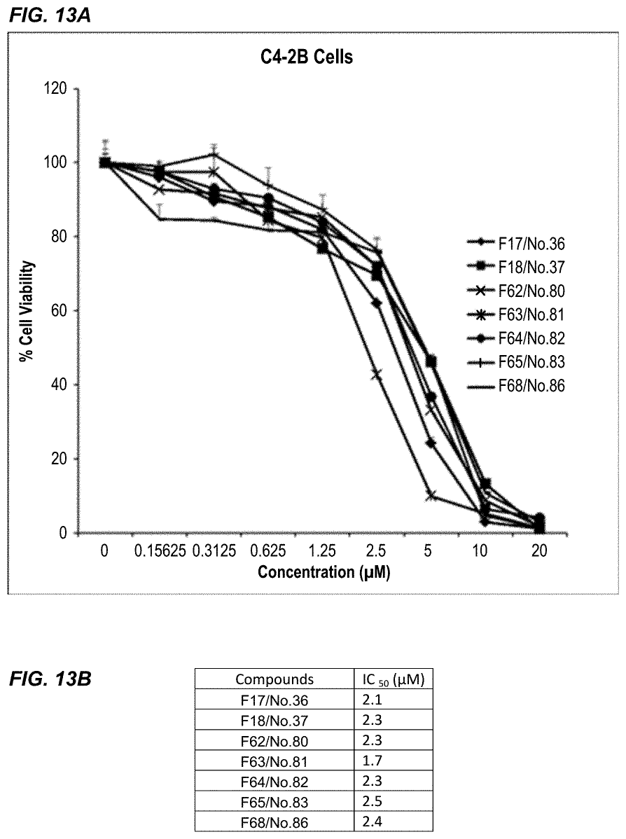

FIGS. 13A-13B show that the RORγ antagonists/inhibitors F17 (Structure No. 36), F18 (Structure No. 37; XY018), F62 (Structure No. 80), F63 (Structure No. 81), F64 (Structure No. 82), F65 (Structure No. 83), and F68 (Structure No. 86) strongly inhibited the growth and survival of prostate cancer C4-2B cells. C4-2B cells are derived from human tumors of prostate cancer and are castration-resistant.

### Materials and Methods

For cell viability, cells were seeded in 96-well plates at 1500-2500 cells per well (optimum density for growth, depending on the specific cell line) in a total volume of 100 μl media. Serially diluted compounds in 100 μl of media were added to the cells 12 hours later. After 4 days of incubation, Cell-Titer GLO reagents (Promega) were added and luminescence was measured on GLOMAX microplate luminometer (Promega), according to the manufacturer's instructions. All experimental points were set up as sextuplicate as biological replication and the entire experiments were repeated three times. The data are presented as percentage of viable cells with vehicle treated cells set as 100. The estimated in vitro IC₅₀ values were calculated using GraphPad Prism 6 software.

### Example 7. RORγ Inhibitor Compounds Inhibit Human Gastric Cancer Cell Growth

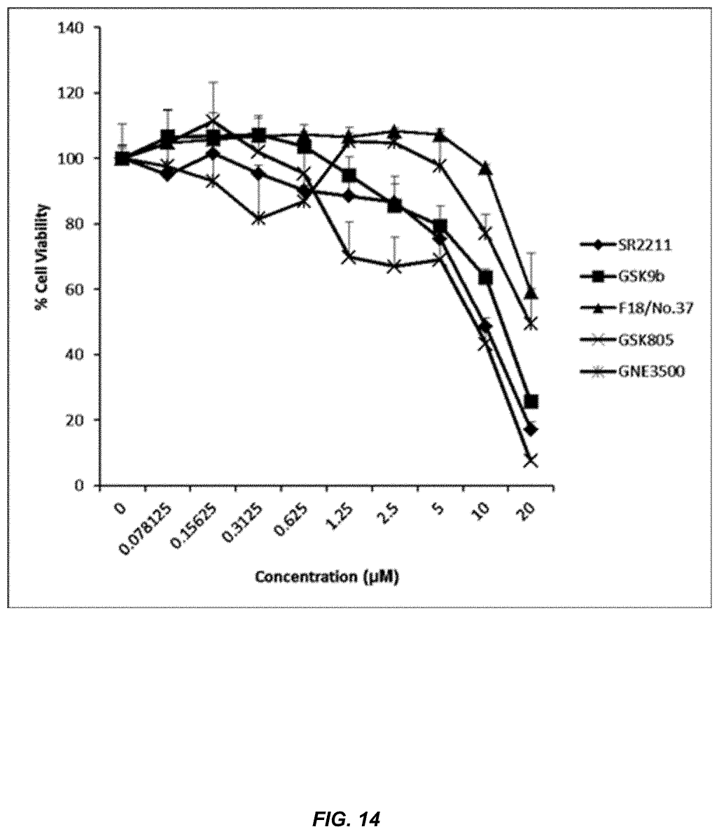

FIG. 14 shows that the RORγ inhibitors/antagonists F18 (Structure No. 37; XY018), GSK9b, GSK805, SR2211, and GNE3500 strongly inhibited the growth and survival of gastric cancer KATO III cells. KATO III cells are derived from human tumors of gastric cancer.

### Materials and Methods

For cell viability, cells were seeded in 96-well plates at 1500-2500 cells per well (optimum density for growth, depending on the specific cell line) in a total volume of 100 μl media. Serially diluted compounds in 100 μl of media were added to the cells 12 hours later. After 4 days of incubation, Cell-Titer GLO reagents (Promega) were added and luminescence was measured on GLOMAX microplate luminometer (Promega), according to the manufacturer's instructions. All experimental points were set up as sextuplicate as biological replication and the entire experiments were repeated three times. The data are presented as percentage of viable cells with vehicle treated cells set as 100.

### Example 8. Series of RORγ Inhibitor Compounds of Formula I Inhibit Human Breast Cancer Cell Growth

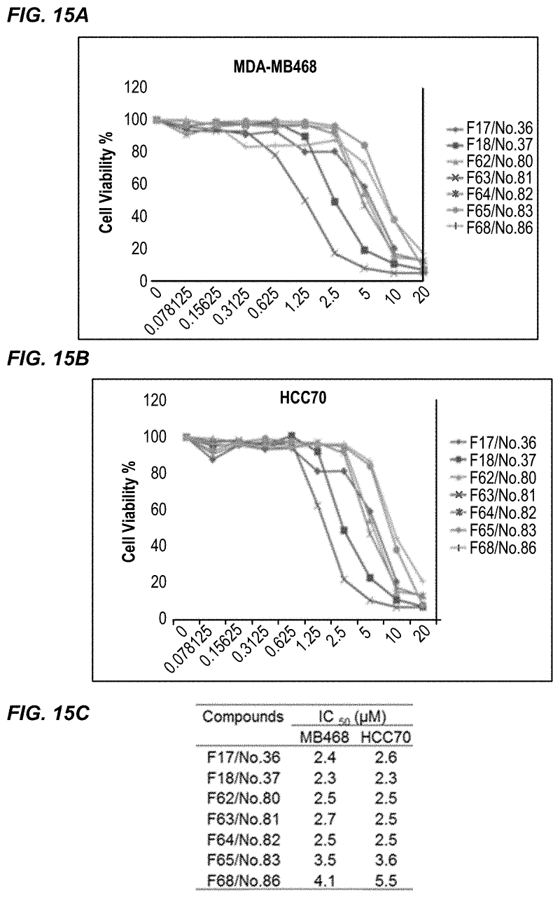

FIGS. 15A-B show that the RORγ antagonists/inhibitors F17 (Structure No. 36), F18 (Structure No. 37; XY018), F62 (Structure No. 80), F63 (Structure No. 81), F64 (Structure No. 82), F65 (Structure No. 83), and F68 (Structure No. 86) strongly inhibited the growth and survival of human triple-negative breast cancer (TNBC) MDA-MB468 and HCC70 cells. These RORγ inhibitors displayed an IC₅₀ ranging from approximately 1 μM to less than 10 μM in TNBC MDA-MB468 and HCC70 (FIG. 15C).

### Materials and Methods

For cell viability, cells were seeded in 96-well plates at 1500-2500 cells per well (optimum density for growth) in a total volume of 100 μl media. Serially diluted compounds in 100 μl of media were added to the cells 12 hours later. After 4 days of incubation, Cell-Titer GLO reagents (Promega) were added and luminescence was measured on GLOMAX microplate luminometer (Promega), according to the manufacturer's instructions. All experimental points were set up as sextuplicate as biological replication and the entire experiments were repeated three times. The data are presented as percentage of viable cells with vehicle treated cells set as 100. The estimated in vitro IC₅₀ values were calculated using GraphPad Prism 6 software.

### Example 9. RORγ Inhibitor Compounds Inhibit Human Breast Cancer Xenograft Tumors

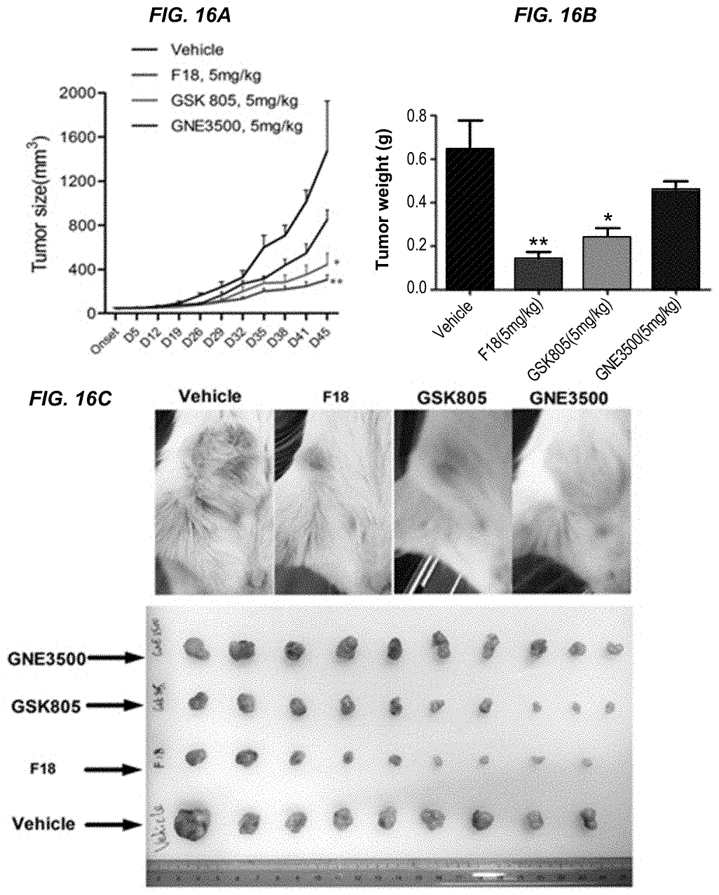

RORγ antagonists/inhibitors were used to evaluate the effect on triple-negative breast cancer (TNBC) SUM159 orthotopic tumor growth in mice. We found that treating mice with 5 mg/kg, i.p., of F18 (Structure No. 37; XY018) effectively inhibited the orthotopic TNBC tumor growth (FIGS. 16A-C). Strong tumor growth inhibition was also observed with other RORγ antagonists/inhibitors such as GSK 805.

### Materials and Methods

Orthotopic xenograft tumor models and chemical compound treatments: Four-week-old female SCID C.B17 mice (for TNBC SUM159 cell lines) were purchased from Harlan Inc. For establishing tumors, 2×10⁶ cells were suspended in total of 100 μL PBS/Matrigel (1:1) and implanted orthotopically into the 4^{th} inguinal mammary fat pads at both sides. Animal group size of six or more was estimated to have a high statistical power, based on power calculation (www.biomath.info/pgwer/) and 20 previous studies involving the same xenograft models. When the tumor volume was approximately 50 mm³, the mice were randomized and then treated intraperitoneally (i.p.) with 100 μl of vehicle or RORγ inhibitors F18/No. 37, 5 mg/kg; GSK805, 5 mg/kg; GNE3500, 5 mg/kg. (in a formulation of 15% Cremophor EL, Calbiochem, 82.5% PBS, and 2.5% DMSO) for five times per week. Tumor growth was monitored by calipers with volume calculated using the equation: π/6 (length×width²). Mouse body weight during the course of the study was also monitored. At the end of the studies, mice were killed and tumors were dissected and weighed.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for treating a cancer in a subject, the method comprising administering to the subject an effective amount of a compound according to Formula I: 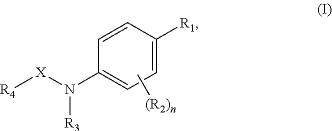
or a pharmaceutically acceptable salt, isomer, racemate, prodrug, co-crystalline complex, hydrate, or solvate thereof, wherein
X is C(═O) or SO₂;
n is an integer selected from the group consisting of 0, 1, 2, or 3;
R₁ is selected from the group consisting of H, halo, alkyl, trifluoromethyl, cyano, —COOR₅, —COR₅, —OR₅, —COH(CF₃)₂, heterocyclyl, and cycloalkyl,
wherein R₅ is selected from the group consisting of H, and C₁-C₃ alkyl group;
R₂ is selected from the group consisting of H, halogen, and alkyl;
R₃ is selected from the group consisting of H and alkyl;
R₄ is selected from the group consisting of C₀-C₄ alkylene-R₆, C₀-C₄ alkylene-R₇-cycloalkyl, and C₀-C₄ alkylene-R₇-heterocyclyl,
wherein R₆ is selected from the group consisting of —R₈, —OR₈, —COR₈, —COOR₈, —S(O)ₘR₈, cycloalkyl, and heterocyclyl, m is 0 or 2, and R₇ is selected from the group consisting of —OR₉, —C(O)R₉, —NR₉, —SR₉, —S(O)R₉, —S(O)₂R₉,
wherein R₈ is selected from the group consisting of H, and C₁-C₃ alkyl group, and R₉ is C₁-C₃ alkylene;
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-, bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of H, C₁-C₄ alkyl, and phenyl, and
R₁₁ is independently selected from the group consisting of C₁-C₄ alkyl, halogen, acetyl, methoxy, and ethoxy.

2. The method of claim 1, wherein the cancer is resistant to an anticancer drug.

3. The method of claim 2, wherein the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof.

4. The method of claim 3, wherein the anti-androgen drug is selected from the group consisting of enzalutamide, bicalutamide, arbiraterone, nilutamide, flutamide, apalutamide, finasteride, dutasteride, alfatradiol, and combinations thereof.

5. The method of claim 3, wherein the chemotherapeutic agent is tamoxifen, a taxane, or combinations thereof.

6. The method of claim 5, wherein the taxane is selected from the group consisting of paclitaxel, docetaxel, and combinations thereof.

7-10. (canceled)

11. The method of claim 1, wherein the compound of Formula I selectively binds to RORγ and inhibits RORγ activity.

12. The method of claim 1, wherein the compound of Formula I s represented by a compound according to any one of Formulas Ic to Ii: 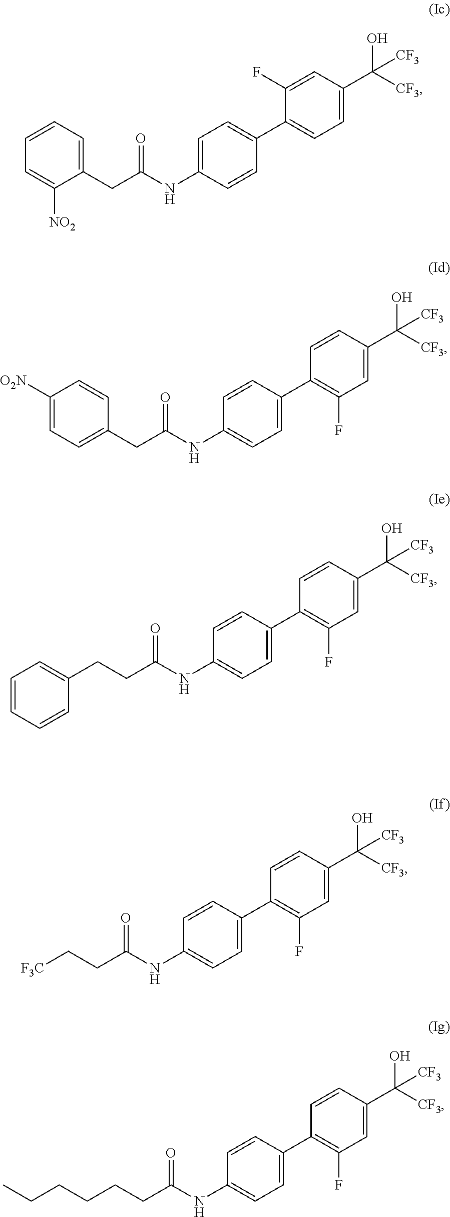 [SMILES: O=C(Cc1ccccc1[N+](=O)[O-])Nc1ccc(-c2ccc(C(O)(C(F)(F)F)C(F)(F)F)cc2F)cc1 ; O=C(Cc1ccc([N+](=O)[O-])cc1)Nc1ccc(-c2ccc(C(O)(C(F)(F)F)C(F)(F)F)cc2F)cc1 ; O=C(CCc1ccccc1)Nc1ccc(-c2ccc(C(O)(C(F)(F)F)C(F)(F)F)cc2F)cc1 ; O=C(CCC(F)(F)F)Nc1ccc(-c2ccc(C(O)(C(F)(F)F)C(F)(F)F)cc2F)cc1 ; CCCCCCC(=O)Nc1ccc(-c2ccc(C(O)(C(F)(F)F)C(F)(F)F)cc2F)cc1] 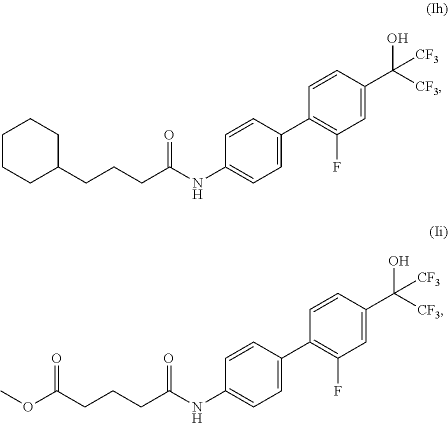 [SMILES: O=C(CCCC1CCCCC1)Nc1ccc(-c2ccc(C(O)(C(F)(F)F)C(F)(F)F)cc2F)cc1 ; COC(=O)CCCC(=O)Nc1ccc(-c2ccc(C(O)(C(F)(F)F)C(F)(F)F)cc2F)cc1]
or a pharmaceutically acceptable salt thereof, a derivative thereof, an analog thereof, or a combination thereof.

13. (canceled)

14. A method for treating a cancer in a subject, the method comprising administering to the subject an effective amount of a compound according to Formula I: 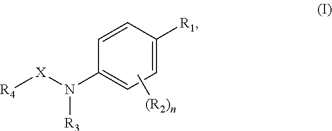
or a pharmaceutically acceptable salt, isomer, racemate, prodrug, co-crystalline complex, hydrate, or solvate thereof, in combination with an effective amount of an anticancer drug, wherein
X is C(═O) or SO₂;
n is an integer selected from the group consisting of 0, 1, 2, or 3;
R₁ is selected from the group consisting of H, halo, alkyl, trifluoromethyl, cyano, —COOR₅, —COR₅, —OR₅, —COH(CF₃)₂, heterocyclyl, and cycloalkyl,
wherein R₅ is selected from the group consisting of H, and C₁-C₃ alkyl group;
R₂ is selected from the group consisting of H, halogen, and alkyl;
R₃ is selected from the group consisting of H and alkyl;
R₄ is selected from the group consisting of C₀-C₄ alkylene-R₆, C₀-C₄ alkylene-R₇-cycloalkyl, and C₀-C₄ alkylene-R₇-heterocyclyl,
wherein R₆ is selected from the group consisting of —R₈, —OR₈, —COR₈, —COOR₈, —S(O)ₘR₈, cycloalkyl, and heterocyclyl, m is 0 or 2, and R₇ is selected from the group consisting of —OR₉, —C(O)R₉, —NR₉, —SR₉, —S(O)R₉, —S(O)₂R₉,
wherein R₈ is selected from the group consisting of H, and C₁-C₃ alkyl group, and R₉ is C₁-C₃ alkylene;
wherein each cycloalkyl group is a saturated or unsaturated ring structure ranging from 3 to 10 carbon atoms, and each cycloalkyl group is optionally substituted with 0, 1, 2 or 3 substituents independently selected from the group consisting of halogen, C₁-C₄ alkyl group, trifluoromethyl, cyano, carboxy, amino, —CONH₂, —COOR₁₀, —COR₁₀, —OR₁₀, —NHCOR₁₀, —NHCOOR₁₀, and —COH(CF₃)₂,
each heterocyclyl group is a 5 to 12 membered saturated or unsaturated mono-, bi- or tri-cyclic structure comprising from 1 to 3 heteroatoms independently selected from the group consisting of N, O, and S, and each heterocyclyl group is optionally substituted with 0, 1, 2 or 3 substituents independently selected from halogen, C₁-C₄ alkyl, trifluoromethyl, cyano, carboxy, nitro, amino, —CONH₂, —COOR₁₀, —COR₁₀, —OR₁₀, —NHCOR₁₀, —NHCOOR₁₀, —COH(CF₃)₂, —C₆H₅R₁₁, morpholinyl, piperidinyl, tetrahydrofuranyl, substituted pyridyl group,
wherein R₁₀ is independently selected from the group consisting of H, C₁-C₄ alkyl, and phenyl, and
R₁₁ is independently selected from the group consisting of C₁-C₄ alkyl, halogen, acetyl, methoxy, and ethoxy.

15. The method of claim 14, wherein the cancer is resistant to the anticancer drug.

16. The method of claim 14, wherein the compound of Formula I enhances the therapeutic effect of the anticancer drug.

17. The method of claim 16, wherein the compound of Formula I reverses or reduces cancer cell resistance to the anticancer drug and/or sensitizes cancer cells to the anticancer drug.

18-21. (canceled)

22. The method of claim 14, wherein the anticancer drug is selected from the group consisting of an anti-androgen drug, chemotherapeutic agent, radiotherapeutic agent, antigen-specific immunotherapeutic agent, endocrine therapy, tyrosine kinase inhibitor, and combinations thereof.

23. The method of claim 22, wherein the anti-androgen drug is selected from the group consisting of enzalutamide, bicalutamide, arbiraterone, nilutamide, flutamide, apalutamide, finasteride, dutasteride, alfatradiol, and combinations thereof.

24. The method of claim 22, wherein the chemotherapeutic agent is tamoxifen, a taxane, or combinations thereof.

25. The method of claim 24, wherein the taxane is selected from the group consisting of paclitaxel, docetaxel, and combinations thereof.

26. The method of claim 14, wherein the compound of Formula I selectively binds to RORγ and inhibits RORγ activity.

27. The method of claim 14, wherein the compound of Formula I is represented by a compound according to any one of Formulas Ic to Ii: 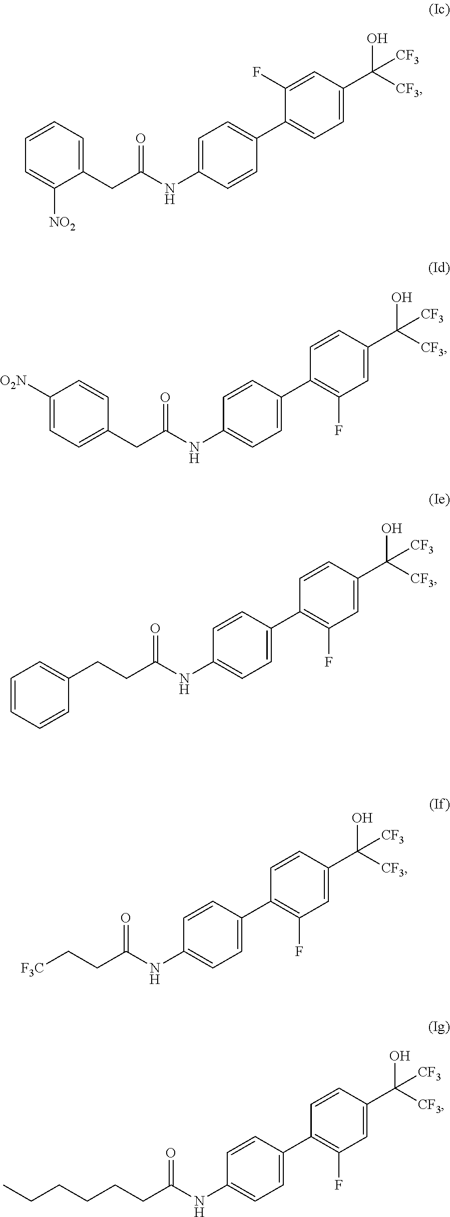 [SMILES: O=C(Cc1ccccc1[N+](=O)[O-])Nc1ccc(-c2ccc(C(O)(C(F)(F)F)C(F)(F)F)cc2F)cc1 ; O=C(Cc1ccc([N+](=O)[O-])cc1)Nc1ccc(-c2ccc(C(O)(C(F)(F)F)C(F)(F)F)cc2F)cc1 ; O=C(CCc1ccccc1)Nc1ccc(-c2ccc(C(O)(C(F)(F)F)C(F)(F)F)cc2F)cc1 ; O=C(CCC(F)(F)F)Nc1ccc(-c2ccc(C(O)(C(F)(F)F)C(F)(F)F)cc2F)cc1 ; CCCCCCC(=O)Nc1ccc(-c2ccc(C(O)(C(F)(F)F)C(F)(F)F)cc2F)cc1] 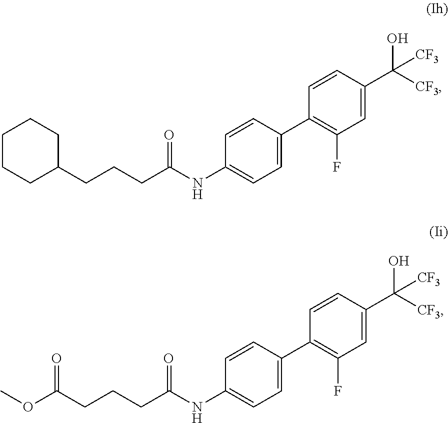 [SMILES: O=C(CCCC1CCCCC1)Nc1ccc(-c2ccc(C(O)(C(F)(F)F)C(F)(F)F)cc2F)cc1 ; COC(=O)CCCC(=O)Nc1ccc(-c2ccc(C(O)(C(F)(F)F)C(F)(F)F)cc2F)cc1]
or a pharmaceutically acceptable salt thereof, a derivative thereof, an analog thereof, or a combination thereof.

28. (canceled)

29. A composition comprising a compound of Formula I and an anticancer drug.

30. (canceled)

31. The composition of claim 29, wherein the compound of Formula I is represented by a compound according to any one of Formulas Ic to Ii: 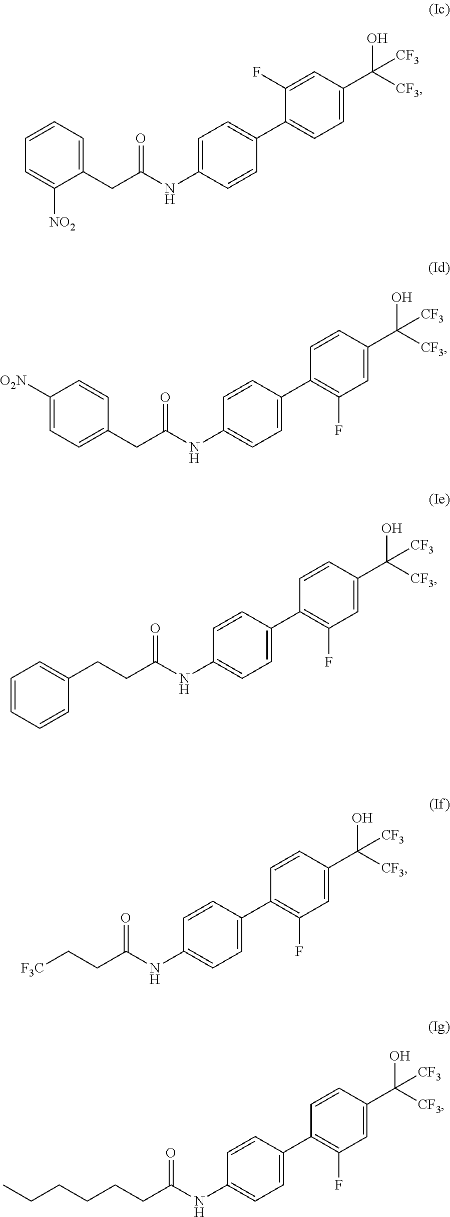 [SMILES: O=C(Cc1ccccc1[N+](=O)[O-])Nc1ccc(-c2ccc(C(O)(C(F)(F)F)C(F)(F)F)cc2F)cc1 ; O=C(Cc1ccc([N+](=O)[O-])cc1)Nc1ccc(-c2ccc(C(O)(C(F)(F)F)C(F)(F)F)cc2F)cc1 ; O=C(CCc1ccccc1)Nc1ccc(-c2ccc(C(O)(C(F)(F)F)C(F)(F)F)cc2F)cc1 ; O=C(CCC(F)(F)F)Nc1ccc(-c2ccc(C(O)(C(F)(F)F)C(F)(F)F)cc2F)cc1 ; CCCCCCC(=O)Nc1ccc(-c2ccc(C(O)(C(F)(F)F)C(F)(F)F)cc2F)cc1] 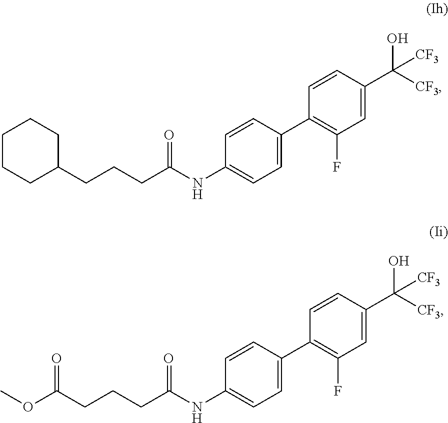 [SMILES: O=C(CCCC1CCCCC1)Nc1ccc(-c2ccc(C(O)(C(F)(F)F)C(F)(F)F)cc2F)cc1 ; COC(=O)CCCC(=O)Nc1ccc(-c2ccc(C(O)(C(F)(F)F)C(F)(F)F)cc2F)cc1]
or a pharmaceutically acceptable salt thereof, a derivative thereof, an analog thereof, or a combination thereof.

32-45. (canceled)


SEQUENCE LISTING (2 sequences)

SEQ ID NO: 1 - protein, 6 aa, Artificial Sequence
  Description of Artificial Sequence Synthetic 6xHis tag
HHHHHH

SEQ ID NO: 2 - protein, 24 aa, Unknown
  Description of Unknown SRC1-4 sequence
QKPTSGPQTPQAQQKSLLQQLLTE